(19) United States
(12) Patent Application Publication
(10) Pub. No.: US 2025/0195637 A1
(43) Pub. Date: Jun. 19, 2025

(54) VACCINE COMPOSITION AGAINST TWO RESPIRATORY VIRUSES

(71) Applicants: VAXXEL, VILLEURBANNE (FR); UNIVERSITE CLAUDE BERNARD LYON 1, VILLEURBANNE (FR); CENTRE NATIONAL DE LA RECHERCHE SCIENTIFIQUE (CNRS), PARIS (FR); ECOLE NORMALE SUPERIEURE DE LYON, LYON (FR); INSERM (INSTITUT NATIONAL DE LA SANTE ET DE LA RECHERCHE MEDICALE), PARIS (FR); UNIVERSITE LAVAL, QUEBEC (CA); Institut national de recherche pour l’agriculture, l’alimentation et l’environnement, PARIS (FR)

(72) Inventors: Julia DUBOIS, BRON (FR); Manuel ROSA-CALATRAVA, LYON (FR); Guy BOIVIN, QUEBEC (CA); Daniela OGONCZYK-MAKOWSKA, QUEBEC (CA); Jean-Francois ELEOUET, JOUY EN JOSAS (FR)

(21) Appl. No.: 18/843,041

(22) PCT Filed: Mar. 1, 2023
(86) PCT No.: PCT/EP2023/055221

§ 371 Date: Aug. 30, 2024

(30) Foreign Application Priority Data
Mar. 2, 2022 (EP) 22305240.8

(51) Int. Cl. A61K 39/155; A61K 39/00; A61P 31/14; C07K 14/005; C12N 7/00

(52) U.S. Cl. CPC A61K 39/155; A61P 31/14; C07K 14/005; C12N 7/00; A61K 2039/5254; A61K 2039/5256; A61K 2039/70; C12N 2760/18321; C12N 2760/18322; C12N 2760/18334; C12N 2760/18362; C12N 2760/18522; C12N 2760/18534

(57) ABSTRACT

The present invention relates to a viral strain derived from the human metapneumovirus (hMPV) strain having a genome sequence represented by sequence SEQ ID NO. 1, wherein said genome sequence comprises the following genetic modifications: (i) inactivation of the endogenous gene coding for the SH protein and/or for the G protein, and (ii) presence of an exogenous nucleotide sequence coding for at least one extracellular domain of the F protein of the human respiratory syncytial virus (hRSV), said domain being wild-type or mutated.

### FIELD OF THE INVENTION

The present invention relates to virus strains genetically modified, comprising antigenic parts from two different respiratory viruses.

The present invention relates to genetically modified viral strains, and their use in the prophylactic or therapeutic treatment of infections with viruses, in particular respiratory viruses.

### INTRODUCTION

Acute infections of the lower respiratory tracts are one of the major causes of morbidity and mortality on the global scale. In children less than 5 years old in particular, they represent the second cause of mortality according to the World Health Organisation.

The majority of acute infections of the lower respiratory tracts, associated with variable pathologies ranging from a simple cold to serious pneumonia, are notably caused by viruses, such as those belonging to the following families:
(i) virus of the Orthomyxoviridae family (notably comprising influenza viruses);
(ii) virus of the Paramyxoviridae family;
(iii) virus of the Adenoviridae family;
(iv) virus of the Picornaviridae family;
(v) virus of the Coronaviridae family and
(vi) virus of the Pneumoviridae family, notably comprising the human syncytial respiratory virus and the human metapneumovirus virus.

The human respiratory syncytial virus (hRSV) and the human metapneumovirus (hMPV) are both responsible for acute infections of the respiratory tracts such as bronchiolitis, bronchitis or pneumonias. They mainly affect populations at risk, which are young children less than 5 years old, the older adults and immuno-compromised persons. Both viruses are present on all continents. Their annual distribution is mainly in winter and spring. Viral transmission essentially takes place by respiratory route, in particular via dispersion in the air of salivary droplets. hRSV was first identified in 1956 in a group of chimpanzees, and then documented to be a mainly human pathogen. It was isolated in 1957. It is a virus with single-stranded RNA genome of negative polarity, of “enveloped” type. Its RNA genome is linear, non-segmented and protected within a helical nucleocapsid. It comprises 10 genes encoding 11 viral proteins. It belongs to the family Pneumoviridae and to the genus Orthopneumovirus. Two hRSV subgroups, A and B, have been identified on the basis of differences of F and G genes.

hRSV is the main etiological agent of bronchiolitis and pneumonias in infants less than 1 year old, with an increased incidence below 6 months old. hRSV is responsible for 6.7% of deaths in children below 1 year of age, and causes excess mortality in the elderly at levels comparable to influenza virus.

hMPV was isolated and described for the first time in 2001 in the Netherlands (van den Hoogen et al., 2001). The following year, several strains of hMPV were isolated in patients located in North America (Peret et al., 2002). hMPV are viruses with negative single-stranded RNA, belonging to the family Pneumoviridae and to the genus Metapneumovirus.

hMPV is prevalent in bronchiolitis and pneumonias in infants, and particularly affects children between 6 months and 3 years old. The average age of children hospitalised due to complications of an infection by hMPV is 6 to 12 months, i.e., later than that caused by hRSV, which mainly occurs between 3 and 6 months.

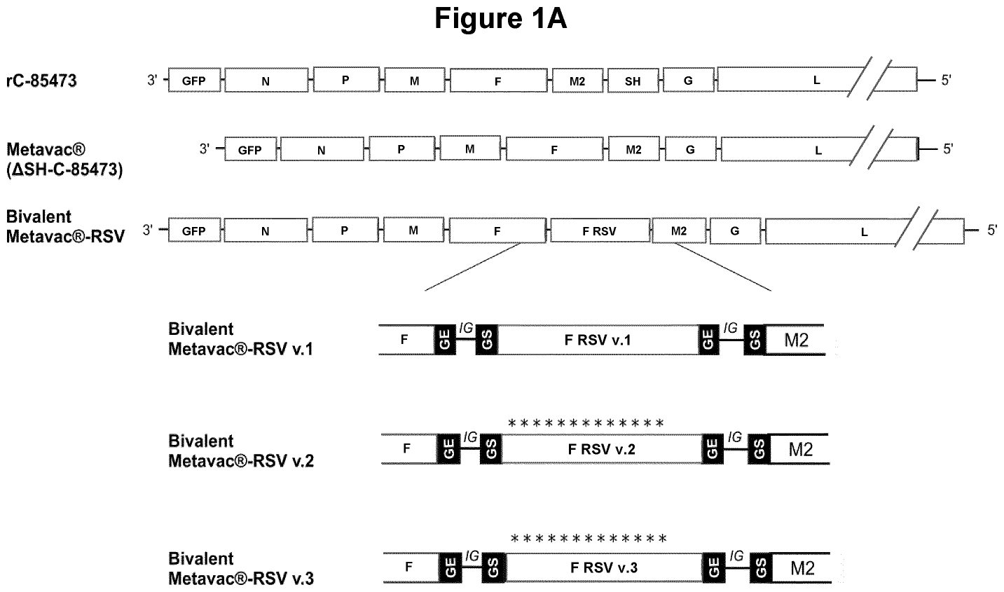

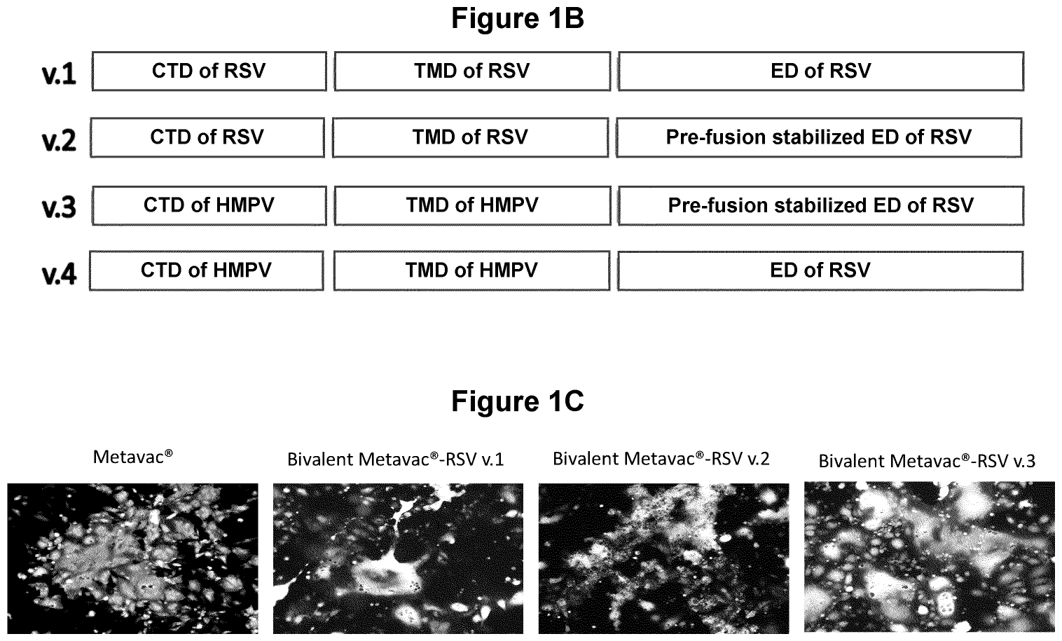

The genomic organisations of hMPV and hRSV are close. Their genome is sub-divided into several ORFs (Open Reading Frames) encoding for N, P, M, F, M2 (M2-1 and M2-2), SH, G and L proteins. hRSV also comprises 2 additional non-structural genes NS1 and NS2, contrary to hMPV. The general genome structure is represented for the virus strain rC-8543 and its attenuated versions Delta SH in FIG. 1.

Three types of glycoproteins are expressed at the surface of the virus particles: F, G and SH proteins.

The F fusion glycoprotein, highly conserved between different subgroups, is involved in the penetration of the virus into the target cells and the formation of syncytium (large multi-nucleated cells derived from the fusion of individual cells following their infection by the virus). The F glycoprotein, also designated as F protein in the present application, is considered as being the most antigenic protein of hRSV and hMPV viruses.

Today, few prophylactic or therapeutic options, specific for hRSV and/or hMPV infection, are available. There is no approved vaccine against either hRSV or hMPV.

For treatment, ribavirin may be used occasionally in serious cases of infections by hMPV or hRSV but this therapy presents several side effects. The usual and widely favoured clinical approach is symptomatic care of the infection: by placing patients under respiratory assistance (administration of oxygen or mechanical ventilation), by administering bronchodilators, corticosteroids and/or antibiotics for preventing or treating bacterial superinfections, and by preventing hypoxemia and electrolyte imbalance.

As a licensed prophylactic treatment, Palivizumab (recombinant humanized hRSV anti-F monoclonal antibody) can be used for hRSV infections, especially for preventing severity of the diseases in high-risk newborns. This preventive treatment is very expensive.

Scientific and clinical communities are currently deeply involved to change this situation of limitation or absence of prophylactic or therapeutic options. Initiatives are underway, such as the creation of RESCEU, a European consortium aiming to regroup all clinical data regarding hRSV infections, and to provide infrastructure to perform trials for future RSV candidate vaccines and therapeutics.

The development of a bivalent vaccine against both hRSV and hMPV thus responds not only a major public health challenge, but also a real socio-economic issue with the objective of reducing the high cost of treatments and hospitalisations associated with these infections. Furthermore, use of such vaccine would present the advantage to decrease the use of antibiotics in the context of bacterial superinfections, and thus limiting the emergence of antibiotics resistances in the population.

### PRIOR ART

Vaccines based on the use of attenuated living virus strains, designated as Live-Attenuated Vaccines (LAVs), have numerous advantages.

First, attenuated living viruses are able to induce a strong immune response in vaccinated patients, because they induce a physiological response similar to a natural infection with the wild virus (although attenuated), which is not the case when an isolated recombinant antigen is administered. Furthermore, in the case of respiratory viruses, these LAVs can be administered by intra-nasal route, and thus mimic the natural entry of the pathogen.

Live-attenuated vaccines broadly stimulate innate, humoral, and cellular immunity, both systemically and locally in the respiratory tract (mucosal immunity).

Because of this strong induced immune response, use of additional adjuvant(s) is usually not necessary.

Finally, this vaccination strategy does not generate any out-of-control inflammatory reaction, as may be the case with inactivated vaccines.

Therefore, it is considered to be the optimal vaccinal strategy for prevention of respiratory viral infections in infants and young children.

The international application WO 2020/021180 discloses an attenuated hMPV strain, obtained from a clinical specimen C-85473, characterised by significant fusogenic capacities, and genetically modified in order to exhibit attenuated virulence, in particular by suppression of expression of the small hydrophobic protein (ASH) and/or the G protein (AG).

Further, the international application WO 2020/120910 discloses a method for producing this attenuated hMPV strain, with infection and culture of a specific avian immortalized cell line. Attenuated strains of hRSV have also been reported. For example, (Rostad et al., 2016) developed an attenuated viral strain: RSV-A2-dNS-ASH-BAF (DB1), with codon deoptimization of genes for non-structural proteins NS1 and NS2, deletion of the SH protein encoding gene, and replacement of the wild-type fusion (F) protein gene with a low-fusion RSV subgroup B F consensus sequence. The same year, an attenuated viral strain named OE4 (RSV-A2-dNS1-dNS2-DSH-dGm-Gsnull-line19F) was engineered and described in (Stobart et al., 2016). This OE4 strain expresses line 19 F protein and is attenuated by codon-deoptimization of non-structural (NS1 and NS2) genes, deletion of the small hydrophobic (SH) gene, codon-deoptimization of the attachment (G) gene and ablation of the secreted form of G. Safety and immunogenicity of an attenuated viral strain RSV/ANS2/A1313/11314L, with a deletion of the interferon antagonist NS2 gene, deletion of codon 1313 of the RSV polymerase gene and a stabilizing missense mutation 11314L, has been shown in RSV-seronegative children (Karron et al., 2020).

Among the most interesting viral antigens, F proteins, involved in the entry of the virus into the target cells and in the formation of syncytium, have been extensively studied. In particular, antibodies against F protein have been generated.

Administration of a monoclonal antibody directed against F protein of hRSV is currently used to prevent the severity of the disease. Administration to children of this humanized monoclonal antibody, Palivizumab, reduces significantly the risk of hospitalization linked to RSV infection in high-risk children.

F protein merges virus and host-cell membranes by using the difference in folding energy between two conformations:
a metastable state adopted prior to virus-cell interaction (pre-fusion state) and
a stable state that occurs after merging of virus and cell (post-fusion state).

It has been noticed that the major epitopes of F protein of hRSV are found in the prefusion state of F protein. For example, antibodies have been developed which target the antigenic site Ø, a metastable site specific to the prefusion state of the F glycoprotein. These antibodies are 10 to 100-fold more potent than palivizumab, which recognizes the antigenic site II located in pre-fusion and post-fusion state.

Soluble variants of F protein which stably expose the antigenic site Ø have been engineered (Mclellan et al., 2013). These variants consist in the extracellular domain of the F protein, stabilized in the prefusion state.

In the development of live-attenuated vaccines, another field of research is the engineering of bivalent vaccines, comprising antigens from two different pathogens.

Bivalent vaccines based on combination of hRSV and hMPV are described, for example, in the European patent application EP 3 868 874. In these recombinant viruses, based on a rearranged viral strain of hRSV, a gene encoding the F protein of another virus, in particular belonging to the family Pneumoviridae, is (i) inserted between the genes encoding the G protein and the F protein of hRSV, or (ii) replaces the gene encoding the F protein of hRSV.

Nevertheless, efforts for providing safe and efficacious bivalent vaccines against both hMPV and hRSV are still underway. The present invention relates to a novel viral strain with multiple genetic modifications, that is attenuated but still immunogenic, with significant fusogenic capacities. Advantageously, the attenuated phenotype is stable, i.e., non-reversible during the replication cycles. This attenuated viral strain is able to reproduce in vitro on cells, and can be used in vivo as a live-attenuated vaccine allowing to generate an immune response against both hMPV and hRSV in individuals to whom it is administered.

### DESCRIPTION OF THE INVENTION

The present invention concerns a bivalent vaccine, able to generate a multiple immune response in an individual after its administration, against two viruses. Such vaccine induces a combined immune response against two viruses, which reduces the number of injections to an individual for obtaining a preventive immunization against different pathogens.

More particularly, the present invention concerns a viral strain derived from the human metapneumovirus (hMPV)C-85473 strain, having the genome sequence represented in SEQ ID NO. 1, wherein said genome sequence comprises the following genetic modifications:
(i) inactivation of the endogenous gene coding for the SH protein and/or for the G protein, to obtain a strain with attenuated virulence, and
(ii) presence of an exogenous sequence coding for at least the extracellular domain of the F protein of the human respiratory syncytial virus (hRSV), said domain being wild-type or mutated.

In particular, the invention relates to said viral strain comprising an exogenous sequence comprising:
a. a sequence originating from hRSV, coding for at least the extracellular domain of the F protein, said domain being wild-type or mutated; and
b. a sequence originating from hMPV, coding for at least one domain of the F protein, said domain being chosen among the cytoplasmic domain and the transmembrane domain, said domain being wild-type or mutated, said exogenous sequence encoding a chimeric hRSV/hMPV F protein.

Another object of the invention is a genetic cassette comprising one of the following nucleotide sequences:
a sequence coding for a mutated extracellular domain corresponding to the stabilized prefusion state of the F protein of hRSV, and a sequence coding for the wild-type cytoplasmic domain and the wild-type transmembrane domain of the F protein of hMPV, in particular consisting in a sequence as shown in SEQ ID NO.8;
a sequence coding for a wild-type extracellular domain of the protein of hRSV, and a sequence coding for the wild-type cytoplasmic domain and the wild-type transmembrane domain of the F protein of hMPV, in particular consisting in a sequence as shown in SEQ ID NO.9.

The present invention also concerns a viral strain derived from a human metapneumovirus (hMPV) strain comprising a genetic cassette as defined above. Preferentially, this viral strain presents an attenuated virulence.

The invention also relates to said viral strain, for use as a medicament.

The invention also relates to said viral strain, for use in preventing and/or treating infections by at least one respiratory virus, in particular at least, and more particularly two viruses of the Pneumoviridae family.

The invention also relates to a vaccine composition comprising, in a pharmaceutically acceptable vehicle, at least one viral strain such as defined above, and optionally an adjuvant.

### DESCRIPTION OF THE FIGURES

FIG. 1. Structure of hMPV genome and of exogenous F proteins, and infected cells with said viral strains
[1A] Structure of the hMPV rC-85473 genome strain, structure of the attenuated Metavac® strain (ΔSH rC-85473 strain, with genetic modifications intended to attenuate the virulence of the strain by the deletion of the SH gene), and ΔSH rC-85473 strain with introduction of an exogenous coding sequence encoding v.1, v.2 and v.3 constructions. A cassette encoding the green fluorescent protein (GFP) is inserted in 3′ position.
[1B] Schematic representation of additional F RSV antigen to be expressed by the Metavac® recombinant viruses. Structure of the v.1, v.2, v.3 and v.4 proteins encoded by exogenous sequences introduced into the genome of ΔSH rC-85473. v.3 and v.4 are chimeric proteins comprising domains from F proteins originating from hMPV and hRSV viruses.
CTD: cytoplasmic domain; TMD: transmembrane domain; ED: extracellular domain.
[1C] Fluorescent microscopy pictures of infected cells with Metavac® strain and bivalent Metavac®-RSV v.1, v.2 and v.3 constructions (×40 magnification on left panel, ×100 magnification on right panel)

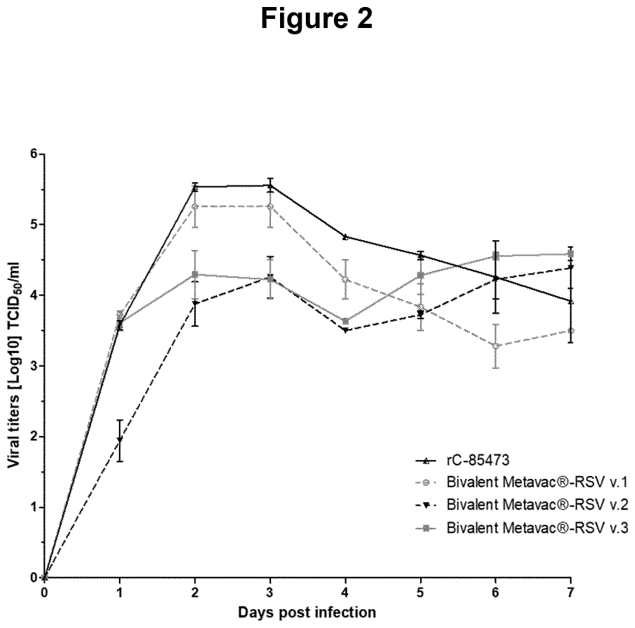

FIG. 2. In vitro replicative capacities of the recombinant bivalent Metavac®-RSV viruses.
LLC-MK2 cells were infected separately, with a multiplicity of infection of 0.01, by the following recombinant viruses:
   rC-85473 strain (full black line);
   Bivalent Metavac®-RSV v.1 (dotted grey line);
   Bivalent Metavac®-RSV v.2 (dotted black line);
   Bivalent Metavac®-RSV v.3 (full grey line).
The cell supernatants were collected each day for 7 days, in triplicate, and viral loads were evaluated by TCID50 assays, which represents the final viral dilution at which 50% of the cell tissue show visible cytopathic effects (50% Tissue Culture Infective Dose).

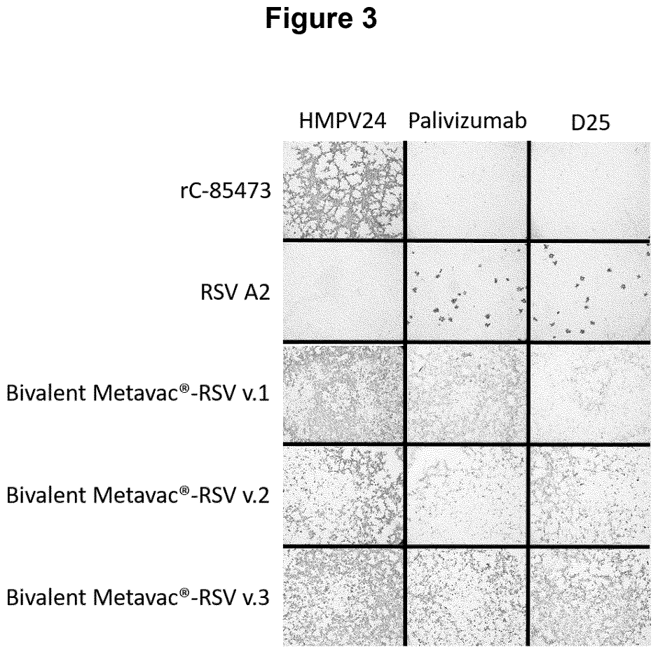

FIG. 3: In vitro expression of exogenous F RSV proteins at the surface of cells infected with the recombinant bivalent Metavac®-RSV viruses.
LLC-MK2 cells were infected (t=0) with the wild-type recombinant rC-85473 HMPV strain, the Bivalent Metavac®-RSV v.1, Bivalent Metavac®-RSV v.2 or with Bivalent Metavac®-RSV v.3, at an MOI of 0.01.
Hep-2 cells (second line) were infected (t=0) with RSV A2 strain at an MOI of 0.01.
After 5 days of infection, infected cell monolayers were fixed in formaldehyde solution and specific immunostainings were performed with:
   HMPV24: Monoclonal mouse antibody detecting the F HMPV protein (MAb HMPV24 BioRad MCA 4674);
   Palivizumab: Monoclonal humanized antibody detecting both pre-fusion and post-fusion forms of the F RSV protein (Palivizumab Synagis® AstraZeneca);
   D25: Monoclonal human antibody detecting the pre-fusion form of the F RSV protein (D25 Mab, Creative Biolabs).

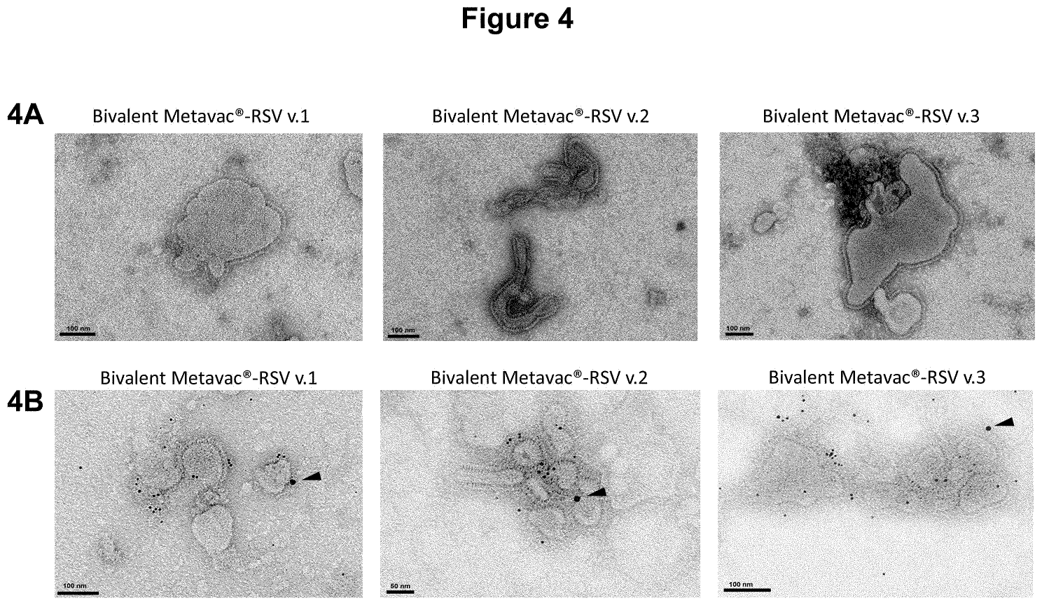

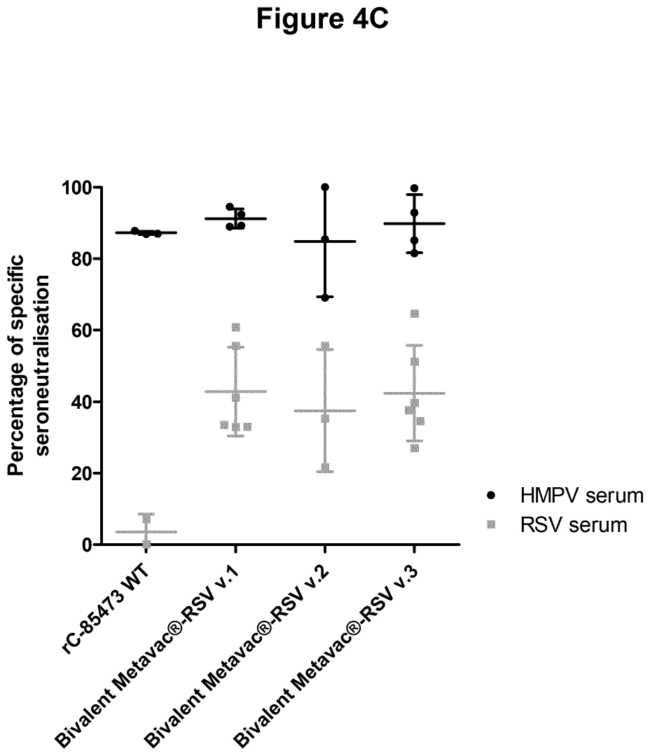

FIG. 4: In vitro expression of exogenous F RSV proteins at the surface of the bivalent Metavac®-RSV particles.
[4A] Observation of the three Bivalent Metavac®-RSV v.1, Bivalent Metavac®-RSV v.2 and Bivalent Metavac®-RSV v.3 viruses by transmission electron microscopy.
[4B] Observation by transmission electron microscopy of the three Bivalent Metavac®-RSV v.1, Bivalent Metavac®-RSV v.2 and Bivalent Metavac®-RSV v.3 viruses co-immunolabelled with monoclonal humanized antibody detecting the F RSV protein (Palivizumab Synagis®) and polyclonal mouse serum detecting HMPV proteins (in house serum).
[4C] Seroneutralization of the three Bivalent Metavac®-RSV v.1, Bivalent Metavac®-RSV v.2 and Bivalent Metavac®-RSV v.3 viruses with specific mouse sera: anti-HMPV (HMPV serum) or anti-RSV (RSV serum).

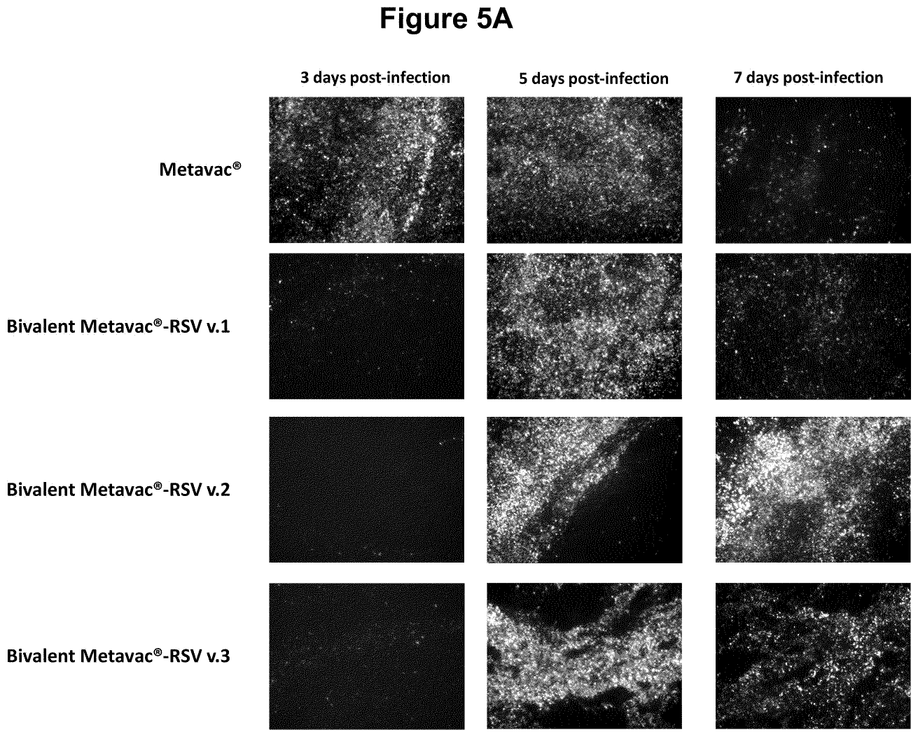

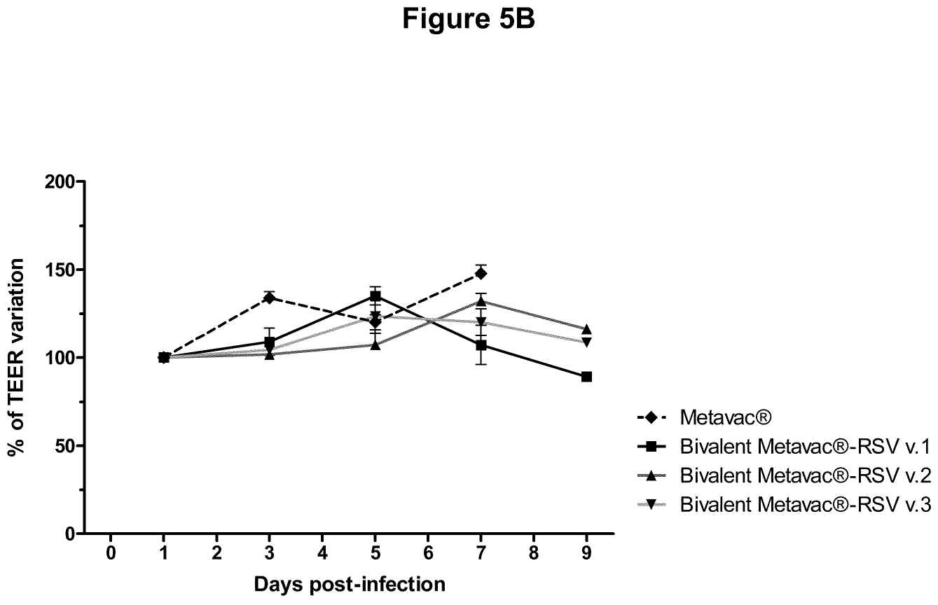

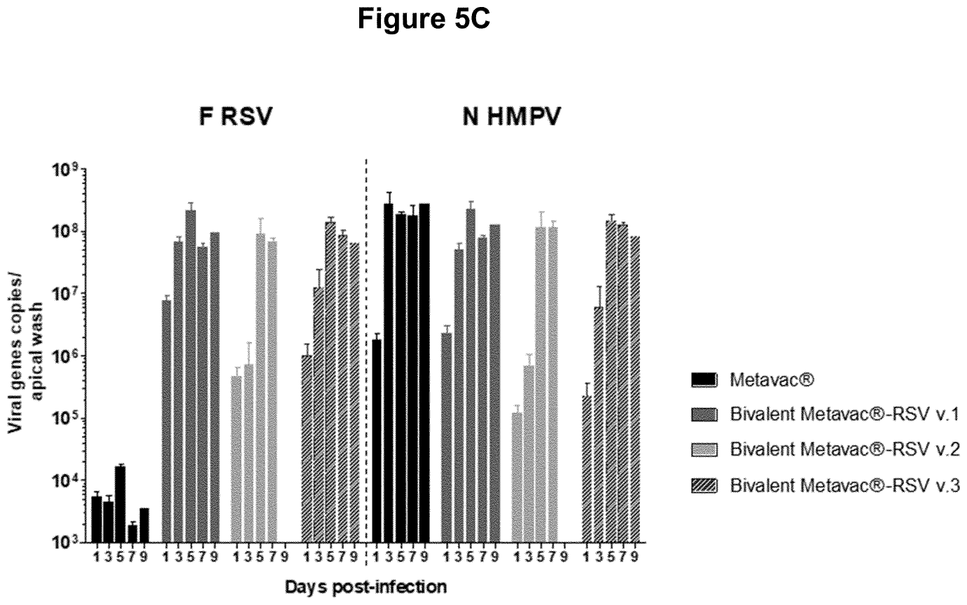

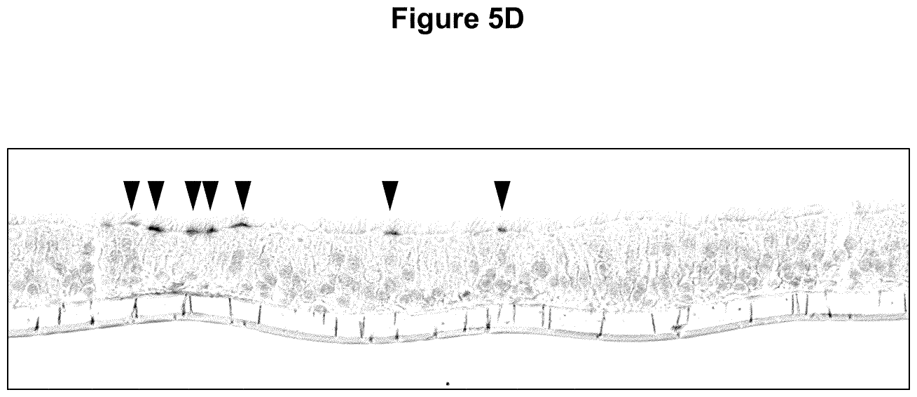

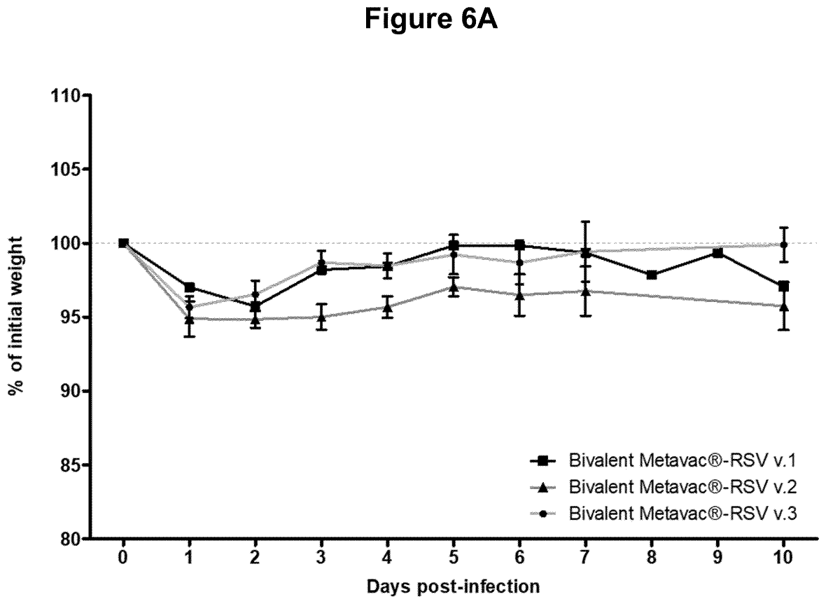

FIG. 5: Infection and replicative capacity of the three bivalent Metavac®-RSV v. 1, v.2 and v. 3 viruses in ex vivo 3D reconstituted human respiratory epithelium.
[5A] Observation in fluorescent microscopy of 3D reconstituted human respiratory epithelia infected with each bivalent Metavac®-RSV v.1, v.2 or v.3 viruses, in comparison to monovalent Metavac® virus. All viruses comprise a GFP encoding gene in 3′ position of their genome.
[5B] Trans-Epithelial Electric Resistance measured during infections of 3D reconstituted human respiratory epithelia with each bivalent Metavac®-RSV v.1, v.2 or v.3 viruses.
[5C] Viral genes quantification at the epithelium apical surface (quantification of the N HMPV gene and the F RSV gene copies performed by RT-qPCR) after 1, 3, 5, 7 or 9 days of infection (post-infection) by the monovalent Metavac® (black bars), bivalent Metavac®-RSV v.1 (dark grey bars), bivalent Metavac®-RSV v.2 (light grey bars) or bivalent Metavac®-RSV v.3 (striped grey bars) viruses. The dotted line represents the threshold of detection.
[5D] Immunolabelling of the F RSV antigen expressed at the surface of 3D reconstituted human respiratory epithelium infected by the bivalent Metavac®-RSV v.1 virus. Immunostaining was performed using Palivizumab (Monoclonal humanized antibody detecting both pre-fusion and post-fusion forms of the F RSV protein). F RSV protein expression at the epithelium apical surface is highlighted by dark arrow heads.

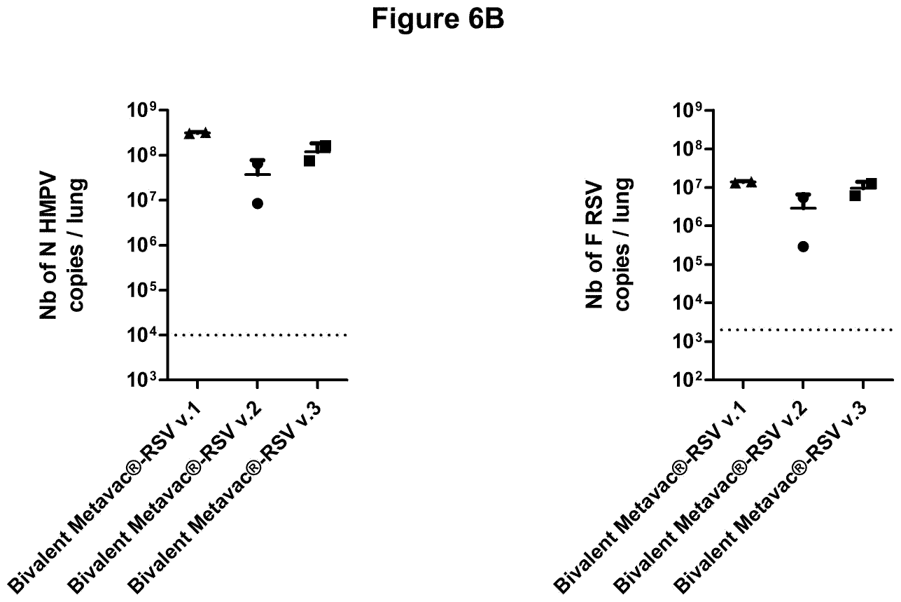

FIG. 6: In vivo characterization of the recombinant bivalent Metavac®-RSV v. 1, v.2 and v. 3 viruses on BALB/c mice viral infection models.
[6A] Weight loss of BALB/c mice during the time-course of the in vivo experiment. Three constructions (Bivalent Metavac®-RSV v.1, v.2 and v.3) are tested.
[6B] Viral genes quantification in the pulmonary tissues after 5 days of infection with Metavac®-RSV v.1, v.2 or v.3: quantification of the N HMPV gene (left graph) and the F RSV gene (right graph) is performed by RT-qPCR. The dotted line represents the threshold of detection.

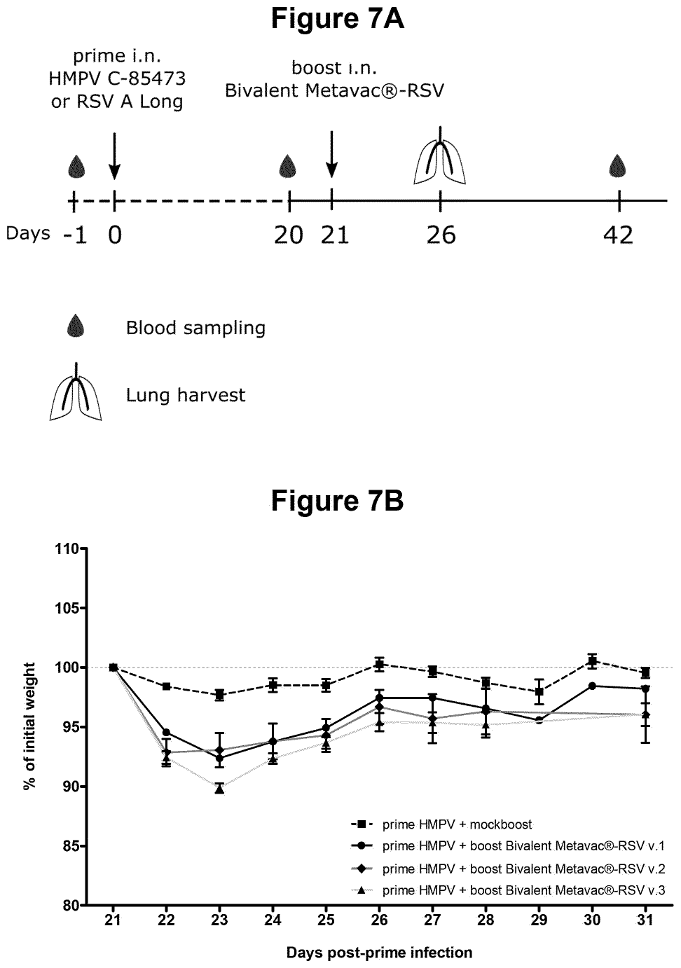

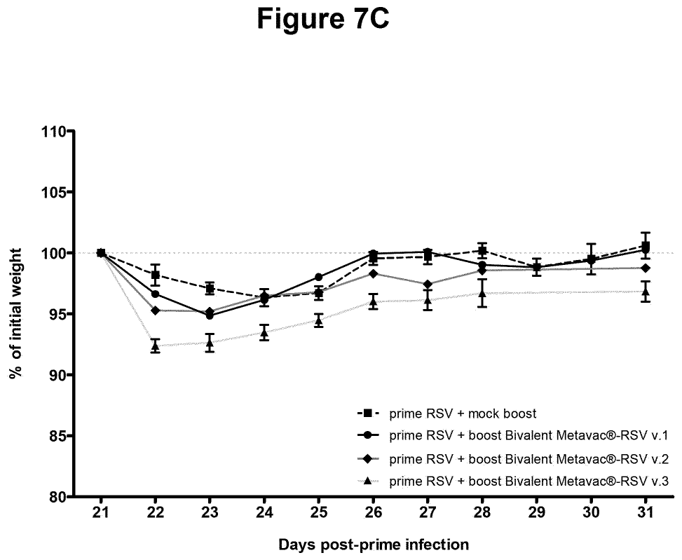

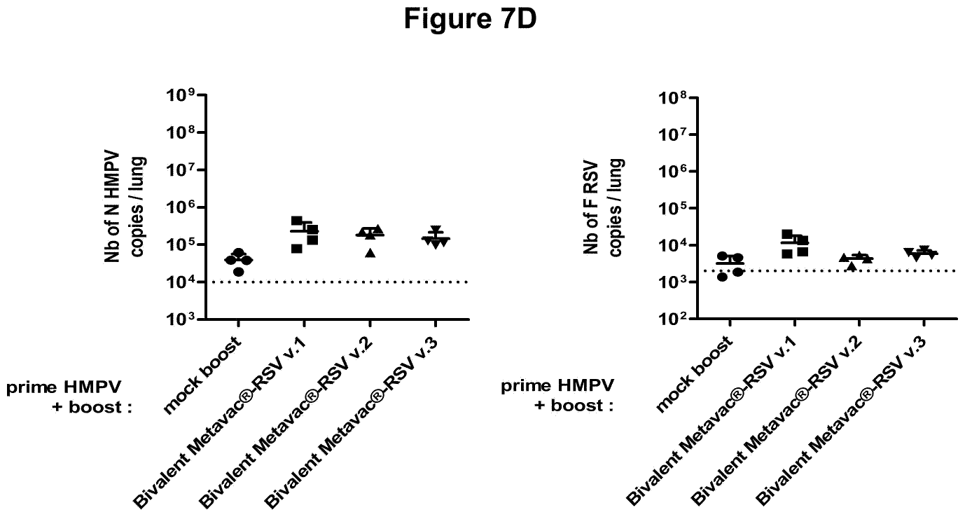

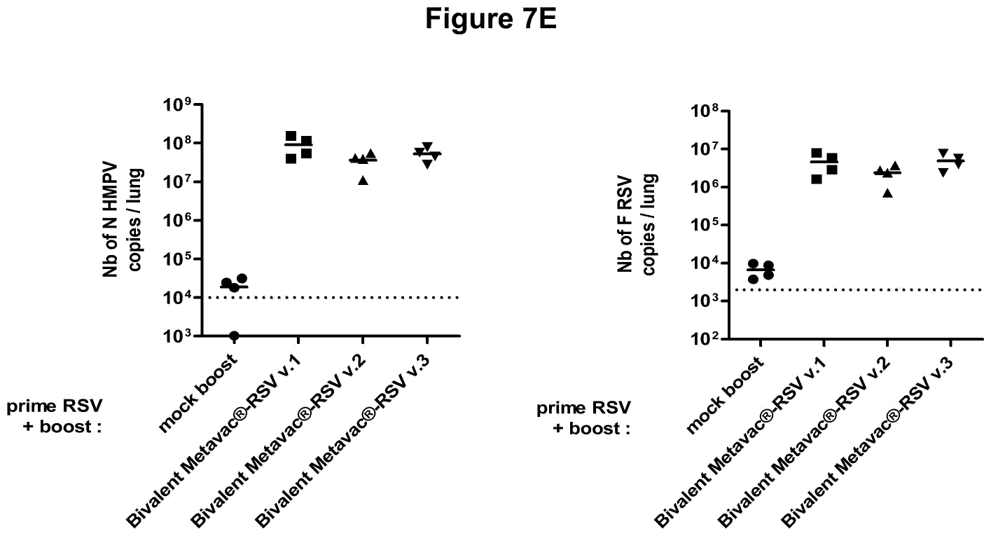

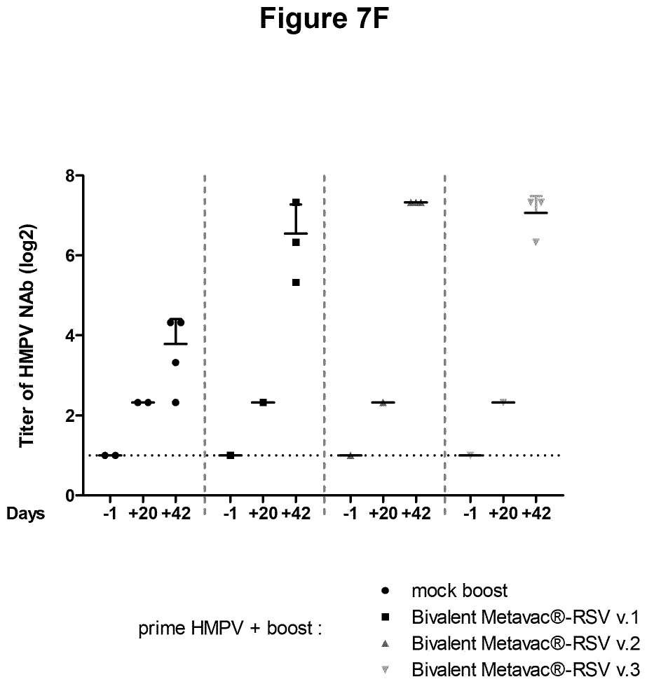

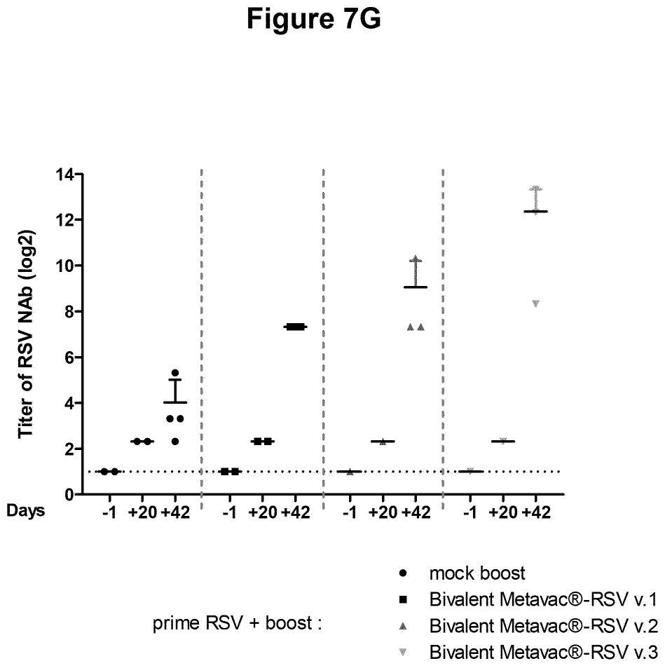

FIG. 7: In vivo induction of neutralizing antibody production after HMPV- or RSV-prime infection followed by boost infection with the bivalent Metavac®-RSV v. 1, v.2 and v.3 viruses on BALB/c mice model.
[7A] Schematic in vivo protocol representation, illustrating a first intranasal instillation of C-85473 HMPV or RSV A wild-type viral strains (prime infection) and a second intranasal instillation of the bivalent Metavac®-RSV v.1, v.2 or v.3 after a three-weeks interval (boost infection), in order to evaluate their potential to induce HMPV- or RSV-specific antibody response in non-naïve BALB/c mice.
[7B] Weight loss of HMPV prime-infected BALB/c mice after an intranasal boost with the bivalent Metavac®-RSV v.1, v.2 or v.3 viruses, in comparison with a group of mice that were boost-instilled with OptiMEM (mock boost).
[7C] Weight loss of RSV prime-infected BALB/c mice after an intranasal boost with the bivalent Metavac®-RSV v.1, v.2 or v.3 viruses, in comparison with a group of mice that were boost-instilled with OptiMEM (mock boost).
[7D] Viral genes quantification in the pulmonary tissues of HMPV prime-infected BALB/c mice 5 days after the intranasal boost with the bivalent Metavac®-RSV v.1, v.2 or v.3 viruses: quantification of the N HMPV gene (left graph) and the F RSV gene (right graph) is performed by RT-qPCR. The dotted line represents the threshold of detection.
[7E] Viral genes quantification in the pulmonary tissues of RSV prime-infected BALB/c mice 5 days after the intranasal boost with the bivalent Metavac®-RSV v.1, v.2 or v.3 viruses: quantification of the N HMPV gene (left graph) and the F RSV gene (right graph) is performed by RT-qPCR. The dotted line represents the threshold of detection.
[7F] Characterization of the HMPV-specific neutralizing antibody response from sera of HMPV prime-infected BALB/c after an intranasal boost with the bivalent Metavac®-RSV v.1, v.2 or v.3 viruses, or a mock boost. Microneutralization assays were performed with sera harvested 1 day before prime-infection (−1), 20 days after prime-infection (+20) and 42 days after prime-infection/21 days after intranasal boost-infection (+42). The dotted line represents the threshold of detection.
[7G] Characterization of the RSV-specific neutralizing antibody response from sera of RSV prime-infected BALB/c mice after an intranasal boost with the bivalent Metavac®-RSV v. 1, v.2 or v.3 viruses, or a mock boost. Microneutralization assays were performed with sera harvested 1 day before prime-infection (−1), 20 days after prime-infection (+20) and 42 days after prime-infection/21 days after intranasal boost-infection (+42), as previously described. The dotted line represents the threshold of detection.

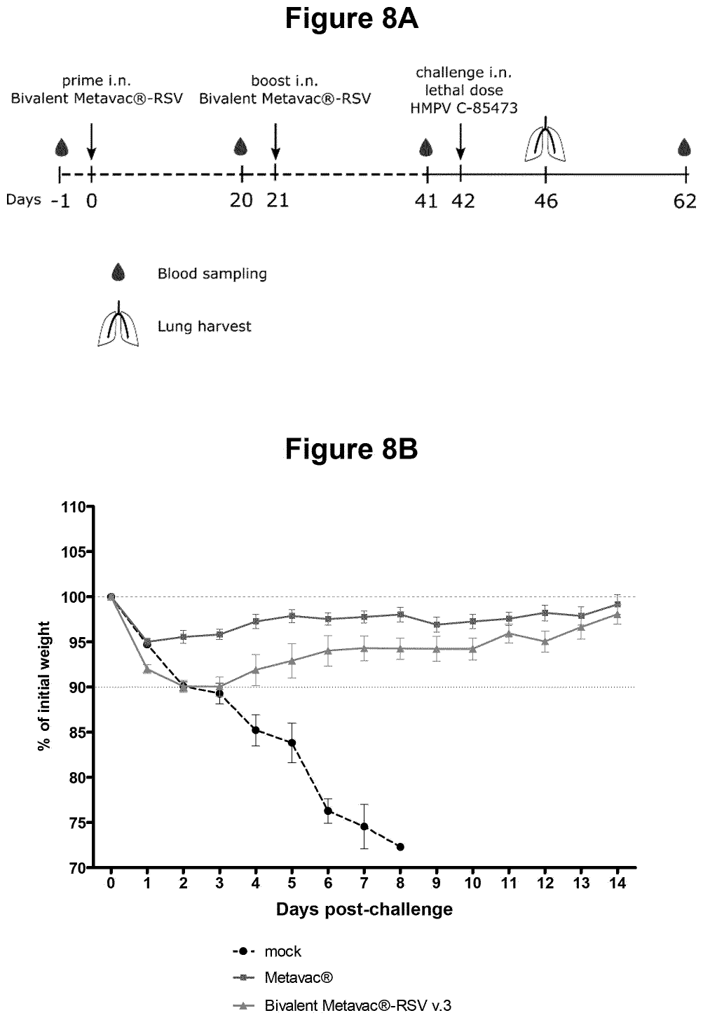

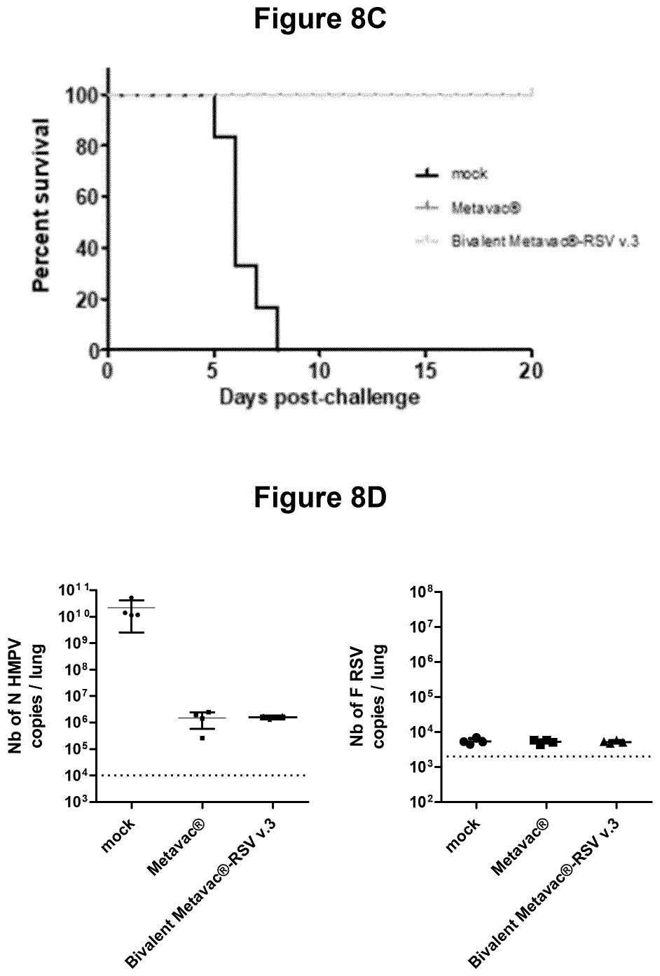

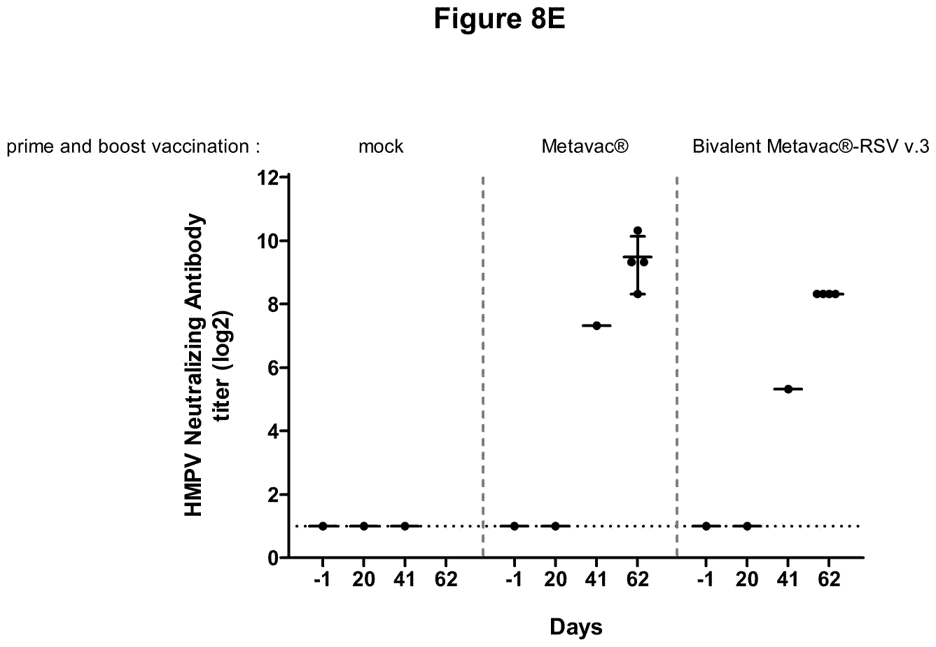

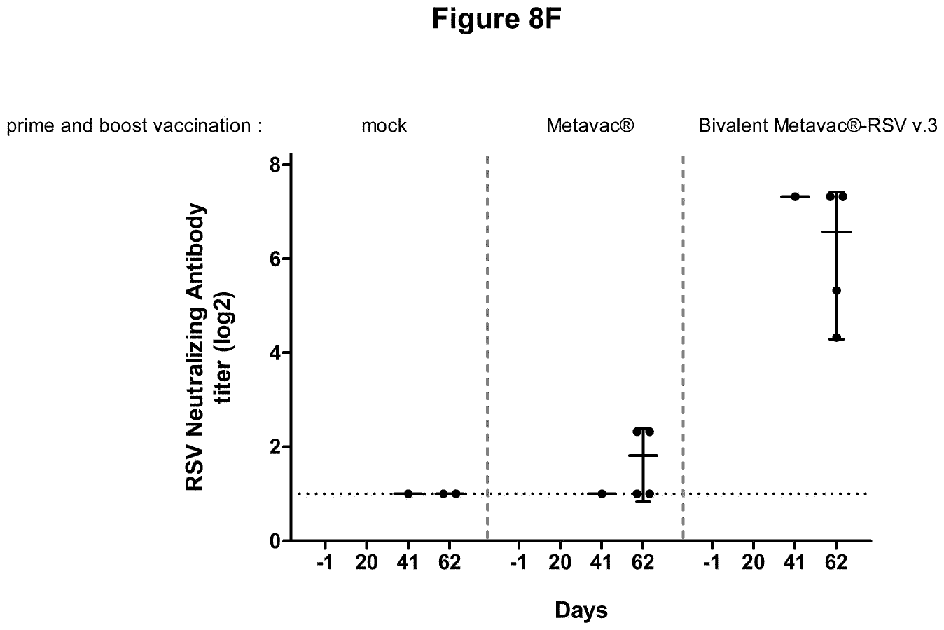

FIG. 8: In vivo protective properties of the bivalent Metavac®-RSV v.3 virus against an infectious challenge with a lethal dose of the wild virus rC-85473 HMPV strain
[8A] Schematic representation of in vivo prime and boost immunization with the bivalent Metavac®-RSV v.3 virus, followed by a lethal viral challenge with a wild-type C-85473 HMPV virus (viral dose expected to induce>50% mortality rate). Each successive infection by intranasal virus instillation is performed after three-weeks interval.
[8B] Weight loss of BALB/c mice after infectious challenge with wild-type HMPV C-85473. Weight of mice prime- and boost-immunized with monovalent Metavac® or bivalent Metavac®-RSV v.3 viruses are compared to a group of mice that were mock-immunized with OptiMEM culture medium.
[8C] Survival percentage of prime- and boost-immunized BALB/c mice after HMPV C-85473 lethal challenge.
[8D] Viral genes quantification within the pulmonary tissues of BALB/c mice prime- and boost-immunized with the bivalent Metavac®-RSV v.3, 5 days after the lethal viral challenge with the wild virus C-85473 HMPV strain: quantification of the N HMPV gene (left graph) and the F RSV gene (right graph) is performed by RT-qPCR. The dotted line represents the threshold of detection.
[8E] HMPV-specific neutralizing antibody detection in sera from BALB/c mice prime and boost-immunizated with OptiMEM culture medium (mock), the monovalent Metavac® or the bivalent MetavacR-RSV v.3, and challenged with the wild-type C-85473 HMPV strain. Microneutralization assays were performed with sera harvested 1 day before prime-infection (−1), 20 days after prime-infection (+20), 41 days after prime-infection/20 days after boost-infection (+41) and 62 days after prime-infection/21 days after lethal viral challenge (+62), as previously described. The dotted line represents the threshold of detection.
[8F] RSV-specific neutralizing antibody detection in serum from BALB/c mice prime and boost-immunizated with OptiMEM culture medium (mock), the monovalent Metavac® or the bivalent Metavac®-RSV v.3, and challenged with the wild-type C-85473 HMPV strain. Microneutralization assays were performed with sera harvested 1 day before prime-infection (−1), 20 days after prime-infection (+20), 41 days after prime-infection/20 days after boost-infection (+41) and 62 days after prime-infection/21 days after lethal viral challenge (+62), as previously described. The dotted line represents the threshold of detection.

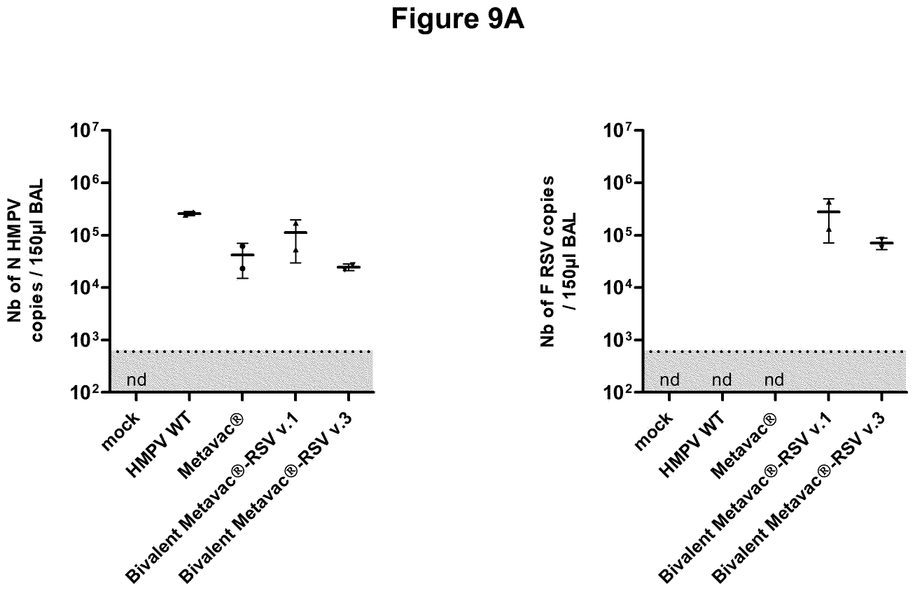

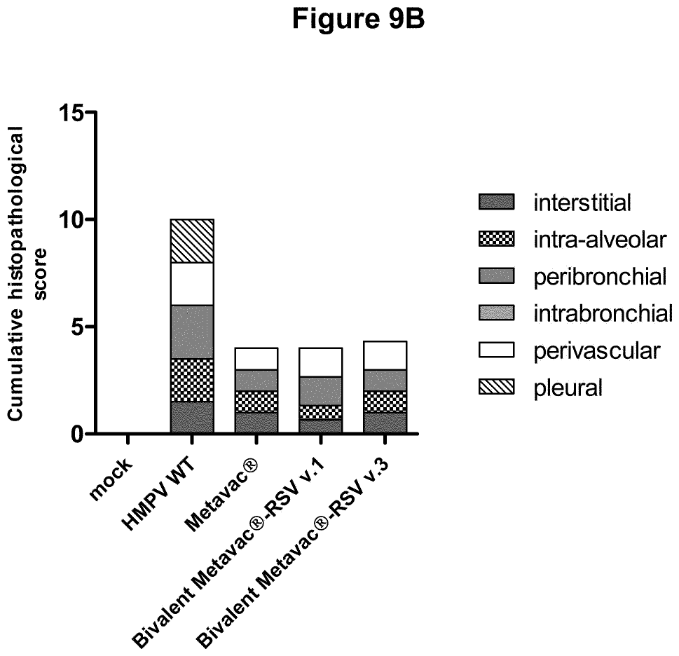

FIG. 9: In vivo characterization of the recombinant bivalent Metavac®-RSV v. 1 and v.3 viruses on BALB/c mice viral infection models.
[9A] Viral genes quantification in broncho-alveolar lavages after 2 days of infection with bivalent Metavac®-RSV v.1 or v.3, in comparison with infection with HMPV WT virus (rC-85437) and monovalent live-attenuated vaccine (LAV) candidate Metavac®: quantification of the N HMPV gene (left graph) and the F RSV gene (right graph) is performed by RT-qPCR. The dotted line represents the threshold of detection.
[9B] Cumulative histopathological score, measured from inflammation estimation in pulmonary tissues (interstitial, intra-alveolar, peribronchial, intrabronchial, perivascular and pleural compartments) of mice infected with HMPV WT virus (rC-85437), monovalent live-attenuated vaccine (LAV) candidate Metavac®, bivalent Metavac®-RSV v.1, bivalent Metavac®-RSV v.3 or «mock»infected. Lungs of infected mice were harvested and fixed in formaldehyde after 5 days of infection.

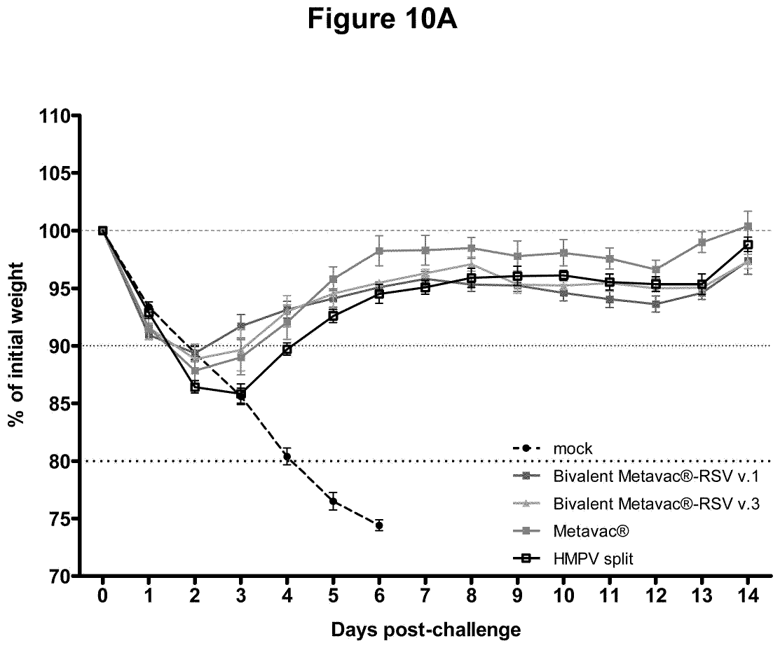

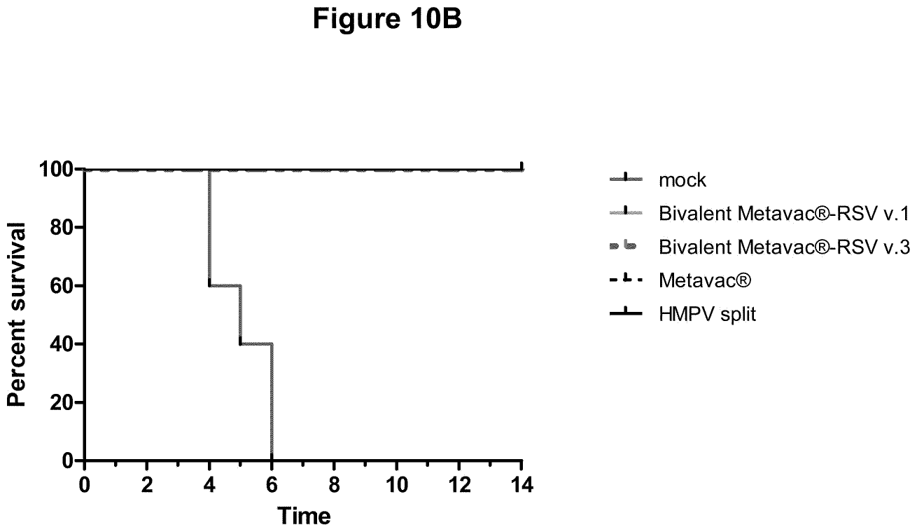

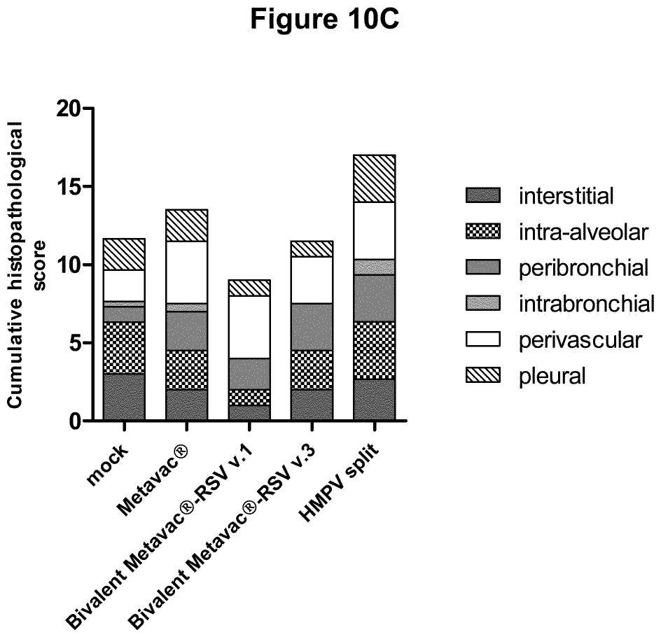

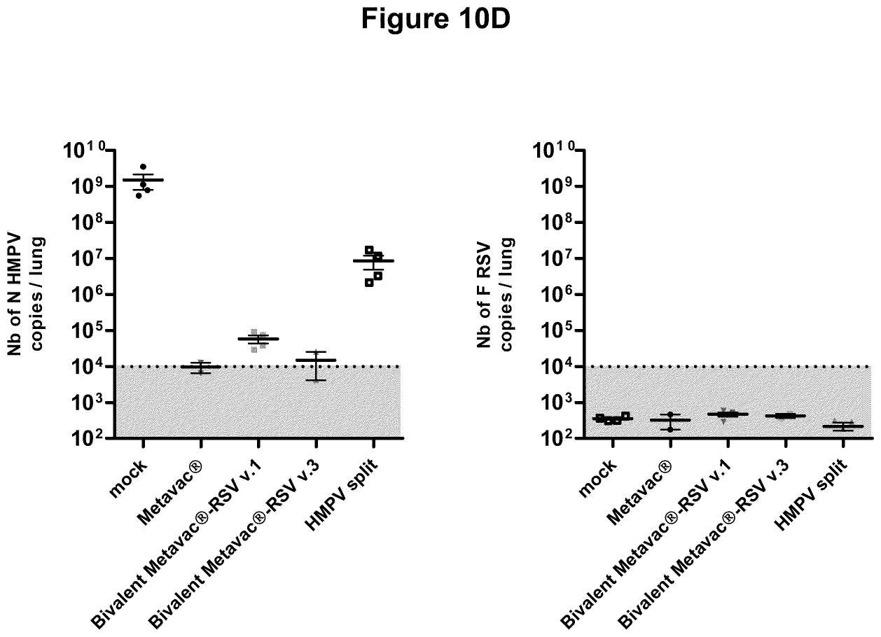

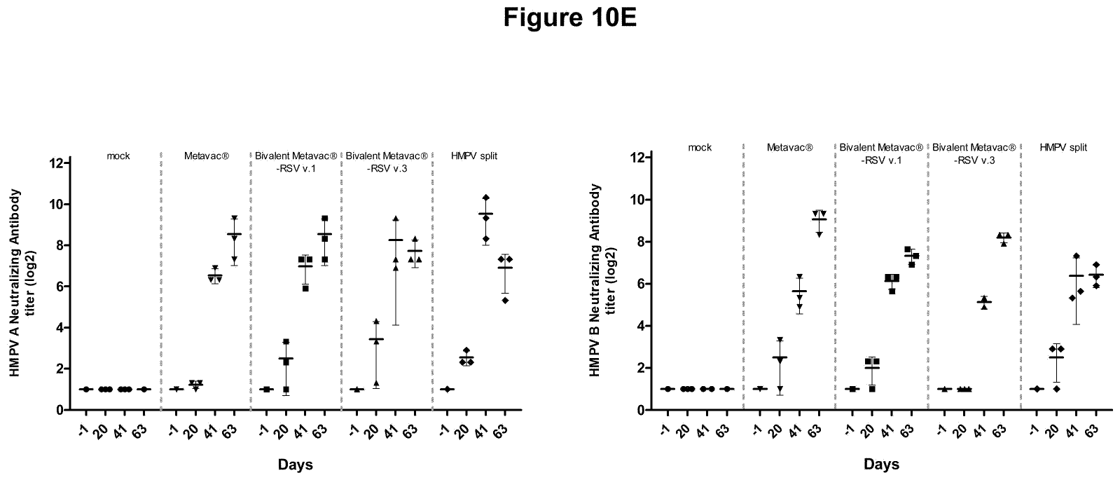

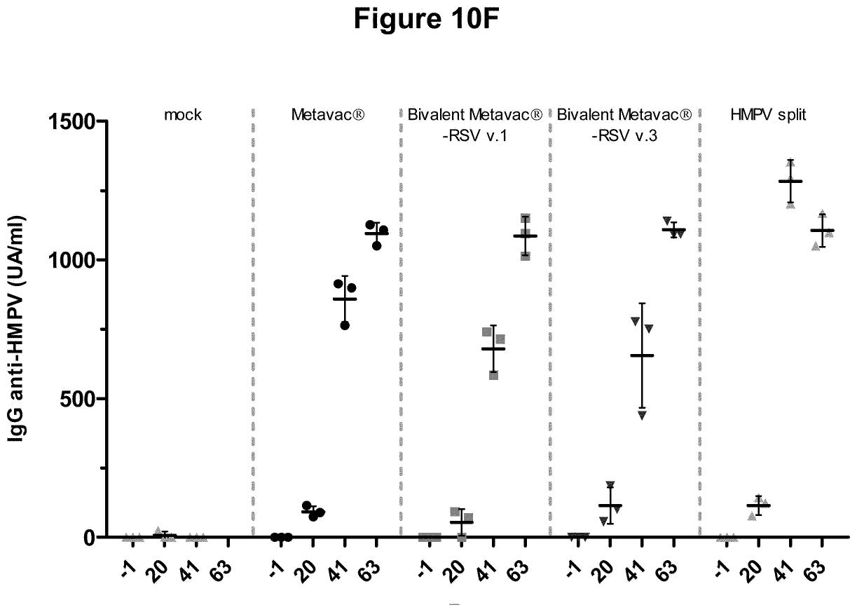

FIG. 10: In vivo protective properties of the bivalent Metavac®-RSV v. 1 and v.3 viruses against an infectious challenge with a lethal dose of the wild virus rC-85473 HMPV strain.
BALB/c mice were immunized twice at 21-days interval via IN route with 5×10⁵ TCID50 of Metavac® or bivalent Metavac®-RSV vaccine candidates V.1 or v.3 or by IM route with HMPV C-85473 viral inactivated split adjuvanted with AddaVax™. Three weeks after the last immunization, animals were inoculated with 2×10⁶ TCID of r C-85473 virus.
[10A] Weight loss of BALB/c mice after infectious challenge with wild-type HMPV C-85473. Weight of mice prime- and boost-immunized with monovalent Metavac®, bivalent Metavac®-RSV v.1 or v.3 viruses are compared to a group of mice that were mock-immunized with OptiMEM culture medium or a group of mice that were immunized via intramuscular route with an adjuvanted split of HMPV WT virus (HMPV split), surrogate of a vaccination with HMPV protein vaccine.
[10B] Survival percentage of prime- and boost-immunized BALB/c mice after HMPV C-85473 lethal challenge.
[10C] Cumulative histopathological score 5 days post-challenge, measured from inflammation estimation in pulmonary tissues (interstitial, intra-alveolar, peribronchial, intrabronchial, perivascular and pleural compartments) of mice prime- and boost-immunized with bivalent Metavac®-RSV v.1, bivalent Metavac®-RSV v.3 or monovalent Metavac® viruses, in comparison with mice mock-immunized or immunized with adjuvanted HMPV split. Lungs of infected mice were harvested and fixed in formaldehyde 5 days after the lethal challenge.
[10D] Viral genes quantification within the pulmonary tissues of BALB/c mice prime- and boost-immunized with the bivalent Metavac®-RSV v.1 or v.3 5 days after the lethal viral challenge with the wild virus C-85473 HMPV strain: quantification of the N HMPV gene (left graph) and the F RSV gene (right graph) is performed by RT-qPCR. The dotted line represents the threshold of detection.
[10E] HMPV-specific neutralizing antibodies detection in sera from BALB/c mice prime and boost-immunizated with OptiMEM culture medium (mock), the monovalent Metavac®, the bivalent Metavac®-RSV v.1 or v.3 or HMPV split, and challenged with the wild-type C-85473 HMPV strain. Microneutralization assays were performed with sera harvested 1 day before prime-infection (−1), 20 days after prime-infection (+20), 41 days after prime-infection/20 days after boost-infection (+41) and 62 days after prime-infection/21 days after lethal viral challenge (+62). Neutralizing antibody titers against homologous HMPV strain (A/rC-85473, left graph) and heterologous HMPV strain (B/CAN98-75, right graph) were measured by specific microneutralization assays.
[10F] HMPV-specific IgG antibodies detection in sera from BALB/c mice prime and boost-immunizated with OptiMEM culture medium (mock), the monovalent Metavac®, the bivalent Metavac®-RSV v.1 or v.3 or HMPV split, and challenged with the wild-type C-85473 HMPV strain were performed using ELISA assay from sera harvested 1 day before prime-infection (−1), 20 days after prime-infection (+20), 41 days after prime-infection/20 days after boost-infection (+41) and 62 days after prime-infection/21 days after lethal viral challenge (+62). IgG titers were represented as arbitrary unit per mL, based on end-point absorbance.

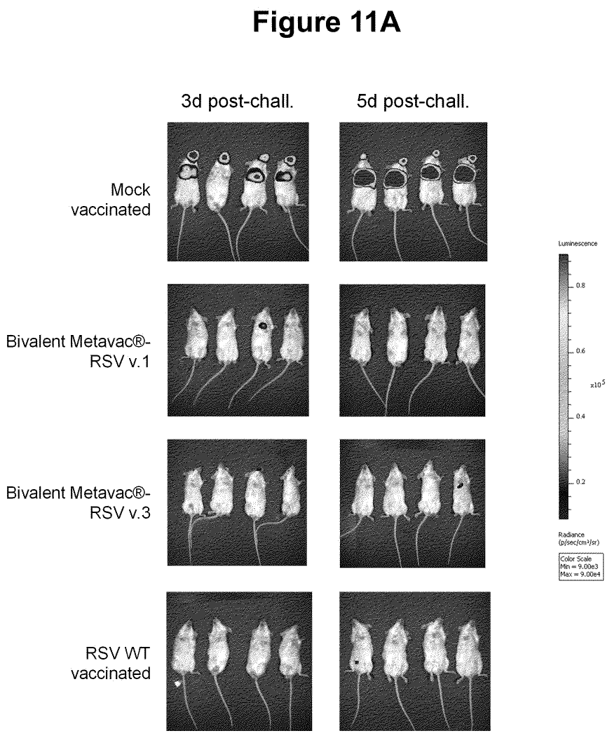

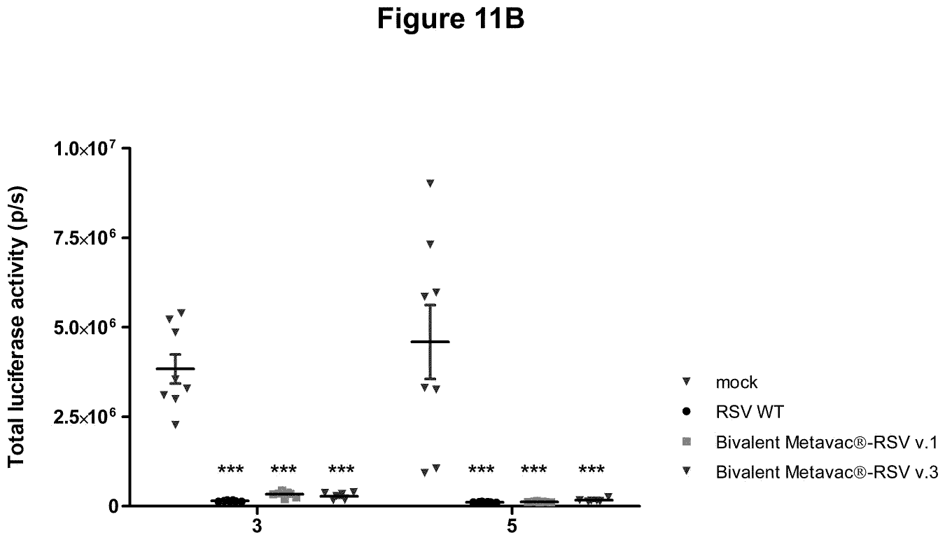

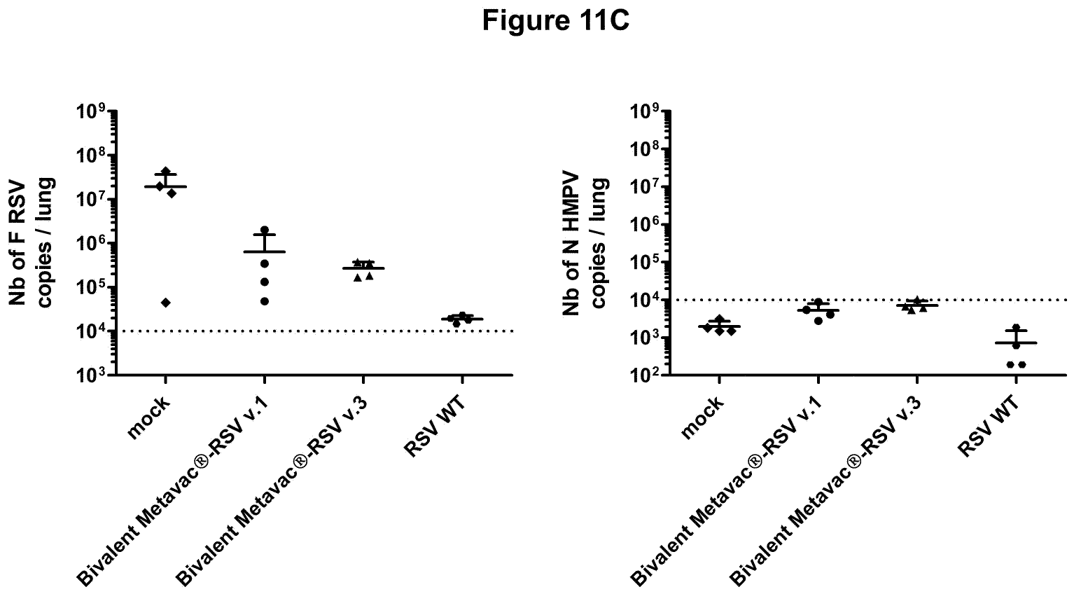

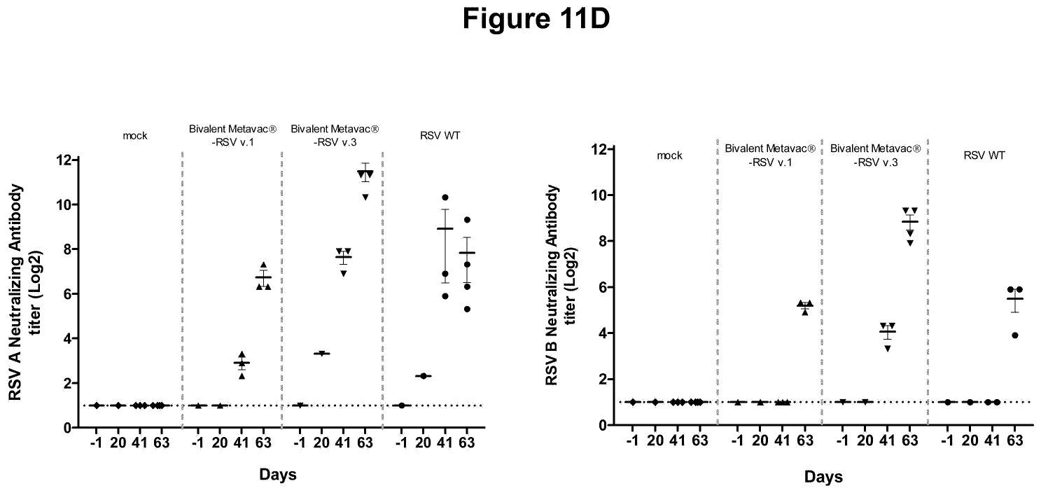

FIG. 11: In vivo protective properties of the bivalent Metavac®-RSV v. 1 and v. 3 viruses against an infectious challenge with a recombinant RSV-Luc WT virus, expressing a luciferase protein.
BALB/c mice were immunized twice at 21-days interval via IN route with 5×10⁵ TCID50 of Metavac®-RSV vaccine candidates or recombinant RSV (RSV WT) virus or mock-immunized. Three weeks after the last immunization, animals were inoculated with 1×10⁵ PFU of rRSV-Luc virus, a recombinant RSV A WT virus expressing a luminescent luciferase protein in vivo.
[11A] Bioluminescence in ventral views of infected mice was imaged at 3 and 5 days post-challenge using an in vivo imaging system (IVIS). Bioluminescence was measured after intranasal injection of 50 μl of D-luciferin. The scale on the right indicates the average radiance (sum of the photons per second from each pixel inside the region of interest, ps⁻¹ cm⁻² sr⁻¹), represented in shades of grey.
[11B] Luciferase activities were quantified from bioluminescence images using ‘Living Image’ software and were represented as mean±SEM photons per second (p/s). Luciferase activities were measured from ventral views of infected mice imaged at 3 and 5 days post-challenge using an in vivo imaging system (IVIS), after intranasal injection of 50 μl of D-luciferin. *** p<0.001 when comparing Metavac®-RSV v.1, v.3 or RSV WT vaccinated group to the mock vaccinated condition using one-way ANOVA with Dunnett's post-test.

[11C] Viral genes quantification within the pulmonary tissues of BALB/c mice prime- and boost-immunized with the bivalent Metavac®-RSV v.1 or v.3 4 days after the viral challenge with the RSV WT virus: quantification of the F RSV gene (left graph) and the N HMPV gene (right graph) is performed by RT-qPCR. The dotted line represents the threshold of detection.
[11D] RSV-specific neutralizing antibodies detection in sera from BALB/c mice prime and boost-immunizated with OptiMEM culture medium (mock), the bivalent Metavac®-RSV v. 1 or v.3 or recombinant RSV WT virus, and challenged with the rRSV-Luc virus. Microneutralization assays were performed with sera harvested 1 day before prime-infection (−1), 20 days after prime-infection (+20), 41 days after prime-infection/20 days after boost-infection (+41) and 62 days after prime-infection/21 days after viral challenge (+62). Neutralizing antibody titers against homologous RSV A strain (RSV A Long, left graph) and heterologous RSV B strain (WV/14617/85, right graph) were measured by specific microneutralization assays.

### DETAILED DESCRIPTION OF THE INVENTION

Human Metapneumovirus (hMPV) Virus Strain

The present invention is based on a virus strain of human metapneumovirus, designated C-85473, isolated from a patient sample in Canada, described in the article (Hamelin et al., 2010).

The recombinant strain rC-85473, originating from this virus strain C-85473 and obtained by reverse genetic engineering, is characterised by considerable fusogenic capacities. Furthermore, rC-85473 is able to penetrate into target cells at a high frequency, i.e., a high degree of infection. Without wishing to be bound by any theory, inventors attribute these properties to a specific sequence of the F protein of rC-85473 strain, which comprises a unique peptide sequence of five amino acids that is not found among the other F proteins of other hMPV strains (Dubois et al., 2017).

This rC-85473 hMPV strain comprises the genomic sequence as shown in SEQ ID NO.1.

The first genetic modification (i) introduced into the rC-85473 strain aims to attenuate the virulence of said strain. An “attenuated virulence” corresponds to an absence of pathogenicity and a reduced inflammatory response in vivo, after administration of an efficient dose of such attenuated viral strain. (Dubois et al., 2019 and Le et al., 2019).

Advantageously, this attenuation of virulence does not affect the replication capacities of the viral strain in vitro, neither its capacities of infection of target cells.

This genetic modification consists in the inactivation of at least one endogenous gene:
the gene coding for the SH protein or
the gene coding for the G protein.

This genetic modification may also be an inactivation of both genes.

In the sense of the invention, the “inactivation of a gene” designates a genetic modification inducing a loss of expression of the gene, or the expression of a non-active form of the encoded protein. This inactivation of a gene may be carried out by all techniques well known to the person skilled in the art. In particular, the inactivation of a gene may be obtained by the introduction of a point mutation into the gene, by the partial or total deletion of the coding sequences of the gene, or by modification of the gene promoter. These different genetic modifications will be carried out according to any one of the molecular biology techniques well known to the person skilled in the art.

In a specific embodiment of the invention, in the viral strain of the invention, the endogenous gene coding for the SH protein is deleted.

In the sense of the invention, “deleted gene” means that a significant part of the coding sequence of this gene has been removed, notably:
a partial deletion of the gene means that at least 50%, 60%, 70%, 80%, 90% or 95% of the coding sequence has been removed;
a complete deletion of the gene means that 100% of the coding sequence has been removed.

According to a preferred embodiment, in the viral strain according to the invention, the gene encoding for the SH protein is completely deleted, that is to say that all (100% of) the coding sequence for the SH protein has been removed from the original genomic sequence. In this case, the viral strain of the invention comprises the nucleotide sequence such as represented in SEQ ID NO. 2. This specific attenuated viral strain is designated in the examples section with the name “Metavac®”, also designated as “monovalent Metavac®”.

A viral strain comprising this first genetic modification (i) is designated below as the attenuated viral strain.

The second genetic modification (ii) introduced into the attenuated rC-85473 strain aims to obtain the expression of an antigen originating from a hRSV strain.

This genetic modification (ii) consists in the introduction of an exogenous coding sequence into the genome of the attenuated viral strain described above, for example comprising a genome sequence such as represented in SEQ ID NO.2.

This introduction of an exogenous coding sequence is not a replacement, but a real addition to the genome of the attenuated rC-85473 strain. In consequence, the F protein of the rC-85473 strain is still present in the genome of the strain, and is expressed, even after the introduction of an exogenous sequence.

Thus, the viral strain of the invention comprises in its genome a sequence coding for at least one extracellular domain of a F protein of the human respiratory syncytial virus (hRSV), said domain being wild-type or mutated.

In the sense of the invention, “exogenous sequence coding for” or “exogenous coding sequence” or “exogenous nucleotide sequence” means a nucleic acid sequence that has been introduced into a viral genome, that is under the control of a suitable promoter, and encodes a protein or a protein domain. In the present case, since the genome of hMPV is made of RNA, the introduced exogenous sequence will also be constituted of RNA. But it is understood that preliminary steps for introducing this exogenous sequence may use corresponding DNA sequence (reverse genetic plasmids).

In the sense of the invention, the term “F protein from hRSV” designates a glycoprotein from a hRSV subgroup A or B, preferentially from a hRSV of subgroup A.

Sequences of all known wild-type F proteins from hRSV can be found in public databases such as UniProt, for example under the access references P11209 or P03420 (precursor forms).

The person of the art knows well the biology of proteins, and can identify the three domains constituting a transmembrane protein: the cytoplasmic domain, inside the cell; the transmembrane domain, inserted into the cell membrane; and the extracellular domain, present at the surface of cell membranes.

In the sense of the invention, the phrase “coding for at least” means that the exogenous sequence codes for at least one peptidic domain, but in most cases also codes for other domains.

In the sense of the invention, the phrase “one extracellular domain of the F protein of hRSV” designates the extracellular domain (expressed at the surface of the viral particles) of any F protein from any hRSV subgroup, and includes wild-type domains and mutated domains.

As is well known by the person of the art, it is preferable to combine an extracellular domain with another peptide sequence allowing its anchoring into a viral particle, in particular at the surface of said viral particle, and/or allowing its anchoring into membranes of infected cells.

Therefore, in a preferred embodiment of the invention, an “exogenous sequence coding for at least the extracellular domain of the F protein of the human respiratory syncytial virus (hRSV), said domain being wild-type or mutated” designates an exogenous sequence coding for a mutated or wild-type extracellular domain of the F protein of hRSV, associated with a sequence coding for at least one anchoring domain, in particular coding for a cytoplasmic and/or a transmembrane domain.

In a first embodiment of the invention, the domain of the F protein is a wild-type domain, i.e., presents the peptide sequence of a domain of a natural F protein from hRSV.

The term “wild-type” designates the typical form of a protein (i.e., its typical peptide sequence) as it occurs in nature. On the contrary, the term “mutated” designates an atypical, non-standard form of the same protein.

In a particular embodiment, the exogenous nucleotide sequence comprises a sequence coding for the wild-type extracellular domain of a hRSV F protein.

In another particular embodiment, the exogenous nucleotide sequence consists in a sequence coding for the wild-type extracellular domain of a hRSV F protein.

In particular, the exogenous nucleotide sequence consists in a sequence coding for the wild-type extracellular domain of the hRSV F protein from a subgroup A virus, more particularly coding for the wild-type extracellular domain of the hRSV F protein having a peptide sequence as shown in SEQ ID NO. 3.

In a particular embodiment, the exogenous nucleotide sequence further comprises a sequence coding for the wild-type transmembrane domain of a hRSV F protein.

In a particular embodiment, the exogenous nucleotide sequence further comprises a sequence coding for the wild-type cytoplasmic domain of a hRSV F protein.

In another particular embodiment, the exogenous nucleotide sequence consists in a sequence coding for the three wild-type domains of a hRSV F protein, i.e., the extracellular, cytoplasmic and transmembrane domains constituting the whole F protein.

In particular, the exogenous nucleotide sequence consists in a sequence coding for the wild-type hRSV F protein from a subgroup A virus, more particularly coding for the wild-type hRSV F protein having a peptide sequence as shown in SEQ ID NO. 4.

In a specific embodiment of the invention, the exogenous nucleotide sequence is integrated into the genome of the attenuated hMPV viral strain at a specific site, for example:
between the gene coding for the N protein and the gene coding for the P protein;
between the gene coding for the P protein and the gene coding for the M protein;
between the gene coding for the F protein and the gene coding for the M2 protein;
between the gene coding for the SH protein and the gene coding for the G protein;
between the gene coding for M2 protein and the gene coding for the G protein (in the case of ΔSH strain);
between the gene coding for G protein and the gene coding for the L protein.

According to one of these specific embodiments, advantageously, the exogenous nucleotide sequence consists in the three wild-type domains of a hRSV F protein, i.e., the extracellular, cytoplasmic and transmembrane domains constituting the whole F protein from hRSV.

A specific chimeric construction comprising the genome of an attenuated hMPV strain (sequence represented in SEQ ID NO. 2) and an exogenous nucleotide sequence coding for a wild-type hRSV F protein (SEQ ID NO. 4) inserted between the gene coding for the F protein and the gene coding for the M2 protein, is designated in the examples section as “Metavac®-RSV v.1”.

The full genomic sequence of this viral strain, combined with the GFP encoding gene, is shown in SEQ ID NO. 10.

In a specific embodiment, the invention concerns a viral strain derived from the human metapneumovirus (hMPV) strain having the genome sequence represented in SEQ ID NO. 1, wherein said genome sequence comprises the following genetic modifications:
(i) inactivation of the endogenous gene coding for the SH protein and/or for the G protein, and
(ii) presence of an exogenous nucleotide sequence coding for a wild-type hRSV F protein of hRSV,
   wherein said exogenous nucleotide sequence is inserted between the gene coding for the F protein and the gene coding for the M2 protein of the hMPV strain.

In a second embodiment of the invention, the domain of the F protein is a mutated domain, i.e., presents a peptide sequence derived from a domain of a wild-type F protein from hRSV comprising at least one point mutation, that is to say the replacement of at least one residue in the wild-type peptide sequence with another residue.

In the context of the present invention, the terms “mutated domain” or “mutated sequence” or “mutated protein” all refer to peptide sequences presenting at least 80% of sequence identity with their corresponding standard, wild-type peptide sequences, and therefore presenting at most 20% of differences with their corresponding wild-type peptide sequences, after optimal alignment of both sequences. In a preferred embodiment, in said mutated domains, the main antigenic epitopes are conserved, i.e., present their wild-type sequence.

In a preferred implementation of the invention, a mutated domain presents at least 80, 81, 82, 83, 84, 85, 86, 87, 88, 89, 90, 91, 92, 93, 94, 95, 96 or 97% of sequence identity with its corresponding wild-type domain. Preferentially, in said mutated domain, the main antigenic epitopes are conserved.

In a more specific embodiment of the invention, a mutated domain corresponds to a mutated domain presenting at least 97% of sequence identity with the corresponding wild-type domain. The percent identities referred to in the context of the present invention are determined on the optimal alignment of the sequences to be compared, which comprise one or more differences such as insertions, deletions, truncations and/or substitutions of amino acids.

This percent identity may be calculated by any sequence analysis method well-known to the person skilled in the art.

The percent identity may be determined after global alignment of the sequences to be compared of the sequences taken in their entirety over their entire length. In addition to manual comparison, it is possible to determine global alignment using the algorithm of Needleman and Wunsch (1970).

For peptide sequences, the sequence comparison may be performed using any software well-known to a person skilled in the art, such as the Needle software. The parameters used may notably be the following: “Gap open” equal to 10.0, “Gap extend” equal to 0.5, and the BLOSUM62 matrix. Preferably, the percent identity is determined via the global alignment of sequences compared over their entire length.

In a particular embodiment, the exogenous nucleotide sequence comprises a sequence coding for a mutated extracellular domain of a hRSV F protein.

In another particular embodiment, the exogenous nucleotide sequence consists in a sequence coding for a mutated extracellular domain of a hRSV F protein.

More preferentially, this mutated domain corresponds to the stabilized prefusion state of the F protein, that is to say has a protein structure restrain under the prefusion state.

This mutated extracellular domain comprises at least one of the following 14 mutations: S46G, K66E, E92D, Q101P, A149C, S155C, S190F, V203L, V207L, S215P, S290C, L373R, Y458C, K465Q, such as described in (Mclellan et al., 2013).

Preferentially, the mutated extracellular domain comprises all 14 mutations listed above. These mutations are incorporated by any technique known by the person of the art, in particular by directed mutagenesis.

In a specific embodiment, this mutated extracellular domain presents a sequence as shown in SEQ ID NO. 5. This mutated domain presents 14 point-mutations over a full length of 513 residues, and therefore has 499 common residues with the corresponding wild-type extracellular domain (residues 1-513 of SEQ ID NO. 4), which corresponds to an identity percentage of 97.27% between the wild-type and the mutated extracellular domain of the F protein of hRSV.

In another embodiment, the exogenous nucleotide sequence consists in a sequence coding for the wild-type cytoplasmic domain, the wild-type transmembrane domain, and a mutated extracellular domain corresponding to the stabilized prefusion state of the F protein of hRSV.

A specific chimeric construction comprising the genome of an attenuated hMPV strain (sequence represented in SEQ ID NO. 2) and an exogenous nucleotide sequence coding for a mutated hRSV F protein, comprising a mutated extracellular domain corresponding to the stabilized prefusion state (SEQ ID NO. 5), is designated in the examples section as “Metavac®-RSV v. 2”.

In particular, the mutated F protein presents the sequence as shown in SEQ ID NO. 6. This mutated protein presents a sequence identity with the wild-type hRSV F protein of 97.5% (560 common residues over a full length of 574 residues).

The full genomic sequence of this viral strain, combined with the GFP encoding gene, is shown in SEQ ID NO. 11.

In another embodiment of the invention, the exogenous nucleotide sequence encodes a chimeric protein comprising domain(s) from F protein of hRSV and domain(s) from F protein of hMPV.

In particular, said exogenous nucleotide sequence encoding a chimeric hRSV/hMPV F protein comprises:
a. a sequence originating from hRSV, coding for at least one extracellular domain of the F protein, said domain being wild-type or mutated; and
b. a sequence originating from hMPV, coding for at least one domain of the F protein, said domain being chosen among the cytoplasmic domain and the transmembrane domain, said domain being wild-type or mutated.

As mentioned earlier, in the context of the invention, the term “mutated domain” refers to domains having peptide sequences having at least 80%, preferentially at least 90%, and more preferentially at least 95%, 96% or 97% of sequence identity with peptide sequences of the corresponding wild-type domains. Preferentially, the main antigenic epitopes of said domain are conserved.

In other words, the invention concerns a viral strain as described above, wherein the exogenous nucleotide sequence encodes a chimeric hRSV/hMPV F protein comprising:
a. a sequence originating from hRSV, coding for at least one extracellular domain of the F protein, said domain being (i) wild-type or (ii) mutated while having at least 80% of sequence identity with the peptide sequence of the corresponding wild-type domain, and
b. a sequence originating from hMPV, coding for at least one domain of the F protein, said domain being chosen among the cytoplasmic domain and the transmembrane domain, said domain being (i) wild-type or (ii) mutated while having at least 80% of sequence identity with the peptide sequence of the corresponding wild-type domain.

Any attenuated viral strain as described above, comprising in its genome an exogenous sequence according to any possible combination of (a) and (b), is an object of the present invention.

For example, said combinations include the following sequences coding for chimeric hRSV/hMPV F proteins consisting in:
a wild-type extracellular and transmembrane domains of F protein from hRSV, and a wild-type cytoplasmic domain of F protein from hMPV;
a wild-type extracellular and cytoplasmic domains of F protein from hRSV, and a wild-type transmembrane domain of F protein from hMPV;
a wild-type extracellular domain of F protein from hRSV, and wild-type transmembrane and cytoplasmic domains of F protein from hMPV;
a mutated extracellular and a wild-type transmembrane domains of F protein from hRSV, and a wild-type cytoplasmic domain of F protein from hMPV;
a mutated extracellular and a wild-type cytoplasmic domains of F protein from hRSV, and a wild-type transmembrane domain of F protein from hMPV;
a mutated extracellular domain of F protein from hRSV, and wild-type transmembrane and cytoplasmic domains of F protein from hMPV.

Advantageously, a “mutated” extracellular domain corresponds to a stabilized prefusion state of the F protein, from hRSV such as described above, in particular presenting at least 97% of sequence identity with the corresponding wild-type domain.

In a specific embodiment, said attenuated viral strain from hMPV comprises an exogenous nucleotide sequence consisting in:
a. a sequence originating from hRSV, coding for a mutated extracellular domain corresponding to the stabilized prefusion state of the F protein of hRSV; and
b. a sequence originating from hMPV, coding for the wild-type cytoplasmic domain and the wild-type transmembrane domain of the F protein of hMPV.

In a specific embodiment, the F protein of hMPV is from a subgroup strain A1.

In another specific embodiment, the F protein of hMPV is from the rC-85473 strain and presents the sequence as shown in SEQ ID NO. 7.

A specific chimeric construction, comprising the genome of an attenuated hMPV strain (sequence represented in SEQ ID NO. 2) and an exogenous nucleotide sequence coding for a chimeric protein, comprising a mutated extracellular domain corresponding to the stabilized prefusion state of F protein from hRSV (SEQ ID NO. 5) and the wild-type cytoplasmic and transmembrane domains of a F protein from hMPV, is designated in the examples section as “Metavac®-RSV v.3”.

In particular, the encoded chimeric protein presents the sequence as shown in SEQ ID NO. 8.

The full genomic sequence of this viral strain, combined with the GFP encoding gene, is shown in SEQ ID NO. 12.

In a specific embodiment, said attenuated viral strain from hMPV comprises an exogenous nucleotide sequence consisting in:
a. a sequence originating from hRSV, coding for a wild-type extracellular domain of the F protein of hRSV; and
b. a sequence originating from hMPV, coding for the wild-type cytoplasmic domain and the wild-type transmembrane domain of the F protein of hMPV.

A specific chimeric construction comprising the genome of an attenuated hMPV strain (sequence represented in SEQ ID NO. 2) and an exogenous sequence coding for a chimeric protein, comprising a wild-type extracellular domain of F protein from hRSV (SEQ ID NO. 3) and the wild-type cytoplasmic and transmembrane domains from the F protein of hMPV, is designated in the examples section as “v.4”.

In particular, the encoded chimeric protein presents the sequence as shown in SEQ ID NO. 9.

In a specific embodiment, the viral strain of the invention presents the following genetic modifications:
(i) inactivation of the endogenous gene coding for the SH protein, and
(ii) presence of an exogenous nucleotide sequence coding for a polypeptide having a sequence chosen among SEQ ID NO. 4, SEQ ID NO. 6, SEQ ID NO.8 and SEQ ID NO.9.
   Chimeric Proteins and Corresponding Coding Sequences, and Viral Strains Comprising them

Another aspect of the invention concerns genetic cassettes encoding chimeric proteins comprising at least one domain from F protein of hRSV and at least one domain from F protein of hMPV.

In particular, said genetic cassette comprises:
a. a nucleotide sequence originating from hRSV, coding for at least the extracellular domain of the F protein, said domain being wild-type or mutated; and
b. a nucleotide sequence originating from hMPV, coding for at least one domain of the F protein, said domain being chosen among the cytoplasmic domain and the transmembrane domain, said domain being wild-type or mutated,
said exogenous sequence encoding a chimeric hRSV/hMPV F protein.

These genetic cassettes also enclose promoter sequences and all regulatory elements allowing the transcription and translation into proteins of the nucleotide sequences (a) and (b).

All possible combinations of (a) and (b) are objects of the present invention.

What is meant by a “mutated domain” has been defined previously. Advantageously, a “mutated” extracellular domain corresponds to the stabilized prefusion state of the F protein, from hRSV. In a specific embodiment, this mutated extracellular domain presents a sequence as shown in SEQ ID NO. 5.

For example, said combinations include the following nucleotide sequences coding for chimeric hRSV/hMPV F proteins consisting in:
a wild-type cytoplasmic domain of F protein from hMPV;
a wild-type extracellular and cytoplasmic domains of F protein from hRSV, and a wild-type transmembrane domain of F protein from hMPV;
a wild-type extracellular domain of F protein from hRSV, and wild-type transmembrane and cytoplasmic domains of F protein from hMPV;
a mutated extracellular and a wild-type transmembrane domains of F protein from hRSV, and a wild-type cytoplasmic domain of F protein from hMPV;
a mutated extracellular and a wild-type cytoplasmic domains of F protein from hRSV, and a wild-type transmembrane domain of F protein from hMPV;
a mutated extracellular domain of F protein from hRSV, and wild-type transmembrane and cytoplasmic domains of F protein from hMPV.

Advantageously, a “mutated” extracellular domain corresponds to a stabilized prefusion state of the F protein, from hRSV.

In a specific embodiment, said genetic cassette comprises the following nucleotide sequences:
a. a sequence originating from hRSV, coding for a mutated extracellular domain corresponding to the stabilized prefusion state of the F protein of hRSV; and
b. a sequence originating from hMPV, coding for the wild-type cytoplasmic and transmembrane domains of the F protein of hMPV.

A specific chimeric construction according to this embodiment is designated in the examples section as “v.3”.

In particular, the encoded chimeric protein presents the sequence as shown in SEQ ID NO. 8.

In a specific embodiment, said genetic cassette comprises the following nucleotide sequences:
a. a sequence originating from hRSV, coding for a wild-type extracellular domain of the F protein of hRSV; and
b. a sequence originating from hMPV, coding for the wild-type cytoplasmic domain and the wild-type transmembrane domain of the F protein of hMPV.

A specific chimeric construction according to this embodiment is designated in the examples section as “v.4”.

In particular, the encoded chimeric protein presents the sequence as shown in SEQ ID NO. 9.

The described nucleotide sequences can be under the form of DNA or RNA.

The genetic cassette described above may be expressed by any system known by the person of the art. For example, a genetic cassette according to the invention can be integrated into a plasmid, into a bacmid, into liposomes, or into any vector of expression. The encoded chimeric proteins may also be used as such.

In another aspect, the invention concerns a viral strain derived from a human metapneumovirus (hMPV) strain, comprising in its genome a genetic cassette such as described above.

Preferentially, the viral strain derived from a hMPV strain is further attenuated, i.e., its virulence is decreased compared to those of the initial viral strain. This attenuation of virulence is obtained, for example, by introducing genetic modifications into the genomic sequence of this viral strain, as is well known by the person of the art.

Viral Strains for their Use Thereof as a Medicine

The present invention also relates to any viral strain as defined above, for its use as a medicament.

Indeed, this attenuated viral strain may be used, notably, for treating and/or preventing infection by at least one respiratory virus, more specifically by at least one virus of the Pneumoviridae family.

In the sense of the invention, the term “treat” designates the fact of combatting infection by a virus in a human or animal organism. In the case of a viral infection, “treating” designates the decrease of the level of viral infection (infectious load) in the organism, and preferably the complete eradication of the virus from the organism. The term “treat” also designates the fact of attenuating the symptoms associated with the viral infection (respiratory syndrome, renal failure, fever, etc.).

In the sense of the invention, the term “prevent” designates the fact of avoiding, or at least decreasing the risk of occurrence, of an infection in an organism. In the case of a viral infection, “preventing” means that the cells of an organism become less permissive to infection, and are thus best placed not to be infected by said virus. It also means that the immune system of the organism has been prepared to react quickly and efficiently in presence of the virus, in order to resist to the infection.

More specifically, the invention relates to a viral strain such as defined above, for use in preventing and/or treating infections by at least one respiratory virus, wherein the at least one respiratory virus is from the Pneumoviridae family, in particular is a human metapneumovirus and/or is the human syncytial respiratory virus.

In particular, the invention concerns a viral strain as described above, for use in preventing infection by two respiratory viruses, a human metapneumovirus (hMPV) and a human syncytial respiratory virus (hRSV).

This viral strain will be preferably integrated in a vaccine composition comprising a pharmaceutically acceptable vehicle, suitable for suspending said viral strain and for the administration thereof.

Said vaccine composition comprises at least one viral strain according to the invention, making it possible to stimulate in a specific manner the immune system of an organism.

Thus, this vaccine composition comprises at least one live attenuated viral strain which plays the role of antigen, that is to say that is recognized and induces a specific immune response in the organism, which will retain the memory thereof.

The present invention also relates to a vaccine composition comprising, in a pharmaceutically acceptable vehicle, at least one viral strain according to the invention, and optionally an adjuvant.

In the sense of the invention, the term “pharmaceutically acceptable vehicle” designates vehicle or excipient, that is to say compound not having any specific action on the infection considered here. These vehicles or excipients are pharmaceutically acceptable, meaning that they may be administered to an individual without risk of significant deleterious effect(s) or prohibitive undesirable effect(s).

The vaccine composition according to the invention comprises at least one effective amount of the viral strain. “Effective amount” is taken to mean, in the sense of the invention, a quantity of viral strain sufficient to trigger an immune reaction in the organism to which it is administered.

The vaccine composition of the present invention is suited for oral, sublingual, inhalation, sub-cutaneous, intramuscular or intravenous administration.

According to a particular embodiment of the invention, the vaccine composition is in a galenic form intended for administration by inhalation.

Inhalation designates absorption by the respiratory tracts. It is in particular a method for absorption of compounds for therapeutic purposes, of certain substances in the form of gas, micro-droplets or powders in suspension.

The administration of pharmaceutical or veterinary compositions by inhalation, that is to say by the nasal and/or buccal passageways, is well known to the person skilled in the art.

Two types of administration by inhalation are distinguished:
administration by insufflation, when the compositions are in the form of powders, and
administration by nebulisation, when the compositions are in the form of aerosols (suspensions) or in the form of solutions, for example pressurised, aqueous solutions. The use of a nebuliser or a spray will then be recommended for administering the pharmaceutical or veterinary composition.

The pharmaceutical form considered here is thus advantageously selected from: a powder, an aqueous suspension of droplets or a pressurised solution.

The target population of respiratory virus is mainly a paediatric population, constituted of individuals less than 18 years old, and more specifically of young children (less than 5 years old) and infants. Administration by inhalation is advantageous, since it is non-invasive.

The invention also relates to a vaccine composition such as described above, for its use for preventing and/or treating infections by at least one respiratory virus, wherein the at least one respiratory virus is from the Pneumoviridae family, in particular is a human metapneumovirus and/or is the human syncytial respiratory virus.

Such a vaccine composition could be used as a preventive vaccine, that is to say intended to stimulate a specific immune response before infection of an organism by a virus.

Such a vaccine composition could also be used as a therapeutic vaccine, that is to say intended to stimulate a specific immune response concomitantly with infection of an organism by said virus.

The present invention also relates to a method for preventing infections by at least one virus from the Pneumoviridae family, in particular hMPV and/or hRSV, comprising the administration to individuals susceptible to be infected by such viruses of a vaccine composition described above.

The present invention also relates to a method for treating an infection with a virus from the Pneumoviridae family, in particular hMPV and/or hRSV, comprising the administration to individuals infected with at least one of these viruses of a vaccine composition described above.

In particular, the individuals are children and infants.

As is shown in the examples section below, the vaccine compositions comprising the viral strains according to the invention induce the production of neutralizing antibodies against multiple strains of hMPV (HMPV A and B, see example 10) and against multiple strains of hRSV (RSV A and B, see example 11).

TABLE 1
Summary of sequences presented in the sequence listing

| | Type | Description |
|---|---|---|
| SEQ ID NO. 1 | DNA | Complete genome sequence of the original strain hMPV rC-85473 |
| SEQ ID NO. 2 | DNA | Complete sequence of the recombinant hMPV ΔSH-rC-85473 |
| SEQ ID NO. 3 | PTR | Extracellular domain of the F protein of hRSV (wild-type) |
| SEQ ID NO. 4 | PTR | Complete peptide sequence of the wild-type F protein from hRSV |
| SEQ ID NO. 5 | PTR | Mutated extracellular domain of the F protein from hRSV, |
| | | corresponding to the stabilized prefusion state of the F protein |
| SEQ ID NO. 6 | PTR | Complete peptide sequence of the mutated F protein from hRSV, |
| | | corresponding to the stabilized prefusion state |
| SEQ ID NO. 7 | PTR | Complete peptide sequence of the wild-type F protein from the |
| | | hMPV strain rC-85473 |
| SEQ ID NO. 8 | PTR | Chimeric protein comprising a mutated extracellular domain of F |
| | | protein from hRSV, and wild-type cytoplasmic and intracellular |
| | | domains of F protein from hMPV |
| SEQ ID NO. 9 | PTR | Chimeric protein comprising a wild-type extracellular domain of F |
| | | protein from hRSV, and wild-type cytoplasmic and intracellular |
| | | domains of F protein from hMPV |
| SEQ ID NO. 10 | DNA | Genome sequence of Bivalent Metavac ® RSV v.1 with GFP |
| | | encoding sequence |
| SEQ ID NO. 11 | DNA | Genome sequence of Bivalent Metavac ® RSV v.2 with GFP |
| | | encoding sequence |
| SEQ ID NO. 12 | DNA | Genome sequence of Bivalent Metavac ® RSV v.3 with GFP |
| | | encoding sequence |
| SEQ ID NO. 13 | DNA | Complete sequence of the recombinant hMPV ΔSH-rC-85473 with |
| | | GFP encoding sequence (corresponding to nucleotides 40 to 784) |

### EXAMPLES

### Example 1. Constructions

Four F RSV protein constructions were designed as represented in FIG. 1A to represent a native F RSV form (v.1), a stabilized pre-fusion F RSV form (v.2), a chimeric HMPV/RSV stabilized F pre-fusion form (v.3) or a chimeric HMPV/RSV native F RSV form (v.4).

F RSV v. 1 sequence corresponds to the native F RSV gene from the RSV A2 strain accessible to the person skilled in the art.

F RSV v.2 sequence corresponds to the F RSV v.1 gene in which 14 mutations (*) has been incorporated by directed mutagenesis. These mutations (S46G, K66E, E92D, Q101P, A149C, S155C, S190F, V203L, V207L, S215P, S290C, L373R, Y458C, K465Q) are described to stabilize the glycoprotein F in its pre-fusion metastable form (Mclellan et al., 2013 doi:10.1126/science.1243283).

F RSV v.3 sequence corresponds to the F RSV v.2 gene in which the region coding for the F RSV protein transmembrane and cytoplasmic domains (amino acid position 514-574) has been replaced by the counterpart coding sequence of the F C-85473 HMPV gene (amino acid position 482-539).

F RSV v.4 sequence corresponds to the F RSV v.1 gene in which the region coding for the F RSV protein transmembrane and cytoplasmic domains (amino acid position 514-574) has been replaced by the counterpart coding sequence of the F C-85473 HMPV gene (amino acid position 482-539).

The corresponding coding sequences were inserted into the plasmid encoding the full-length genome of the rC-85473 HMPV virus (SEQ ID NO. 1).

The insertion of the F RSV coding sequences was performed at several genomic positions, as resumed in Table 2.

Among these conditions, the insertion between the HMPV genes N and P or P and M or F and M2 allowed viral rescue after reverse genetics, following experimental protocols known by the person skilled in the art. HMPV virulence being attenuated by the deletion of the gene encoding for the SH protein (ΔSH), the construction constituted by the insertion of the F RSV coding sequences between F and M2 HMPV genes is the only construction compatible with significant rescue of recombinant viruses, efficient viral propagation and amplification.

TABLE 2
Description and rescue efficacy of
bivalent HMPV/RSV constructions

| F RSV gene position | Flanking HMPV genes | Viral rescue efficacy |
|---|---|---|
| 1A | 3′-GFP | no |
| 1B | GFP-N | no |
| 2 | N-P | yes |
| 3 | P-M | yes |
| 5 | F-M2 | yes |
| 7 | SH-G | yes |

The genetic constructions represented in FIG. 1B were prepared in order that the recombinant viruses are detectable by expression of GFP (Green Fluorescent Protein), have an attenuated virulence by the deletion of the gene encoding for the SH protein (ΔSH, which is named Metavac®, SEQ ID NO. 13 with GFP gene included) and encode for an exogenous viral gene coding the F fusion protein from the Respiratory Syncytial Virus (RSV), the F RSV coding sequence being inserted between HMPV F and M2 genes, in particular three different gene versions as described.

The complete sequences of these genetic constructions are presented in SEQ ID NO. 13 (GFP ΔSH-rC-85473), SEQ ID NO.10 (Bivalent Metavac® RSV v.1), SEQ ID NO.11 (Bivalent Metavac® RSV v.2) and SEQ ID NO. 12 (Bivalent Metavac® RSV v.3).

In the FIG. 1C, LLC-MK2 cells were infected with a multiplicity of infection (MOI) of 0.01 (×40 magnification) by the Metavac® virus or the bivalent generated recombinant viruses: Bivalent Metavac®-RSV v.1, Bivalent Metavac®-RSV v.2 and Bivalent Metavac®-RSV v.3.

The cells are observed by fluorescence microscopy at ten days post-infection.

GFP expression reveals fully functional and replicative recombinant viruses, as well as expected fusogenic phenotype (induction of cellular syncytia via efficient expression of F fusion protein), which are intrinsic characteristics of the attenuated Metavac® recombinant strain.

These results show the capacity of the Metavac® recombinant virus to accept an exogenous F RSV gene leading to the expression of fully functional F fusion proteins and production of propagative and replicative recombinant viruses (rescued by reverse genetic), especially when the corresponding coding sequence is inserted between F and M2 HMPV genes.

### Example 2: In Vitro Replicative Capacities of Three Recombinant Bivalent Metavac®-RSV Viruses

LLC-MK2 cells were infected separately, with a multiplicity of infection of 0.01, by the following recombinant viruses:
rC-85473 strain;
Bivalent Metavac®-RSV v.1;
Bivalent Metavac®-RSV v.2;
Bivalent Metavac®-RSV v.3.

The cell supernatants were collected each day for 7 days, in triplicate, and viral loads were evaluated by TCID50 assays, virology techniques well known to the person skilled in the art, which represents the final viral dilution at which 50% of the cell tissue show visible cytopathic effects (50% Tissue Culture Infective Dose).

The FIG. 2A shows different kinetics, replicative and production capacities in function of the nature of the exogenous F RSV coding sequences inserted into the attenuated Metavac® genetic backbone. The positive control is the non-attenuated rC-85473 strain, which presents similar capacities as the attenuated Metavac® strain.

The recombinant virus Bivalent Metavac®-RSV v. 1 seems to have better replicative capacities than those of the viruses Bivalent Metavac®-RSV v.2 and Bivalent Metavac®-RSV v.3.

In the table below (table 3) are represented the average loads of the viral stocks produced and concentrated for each recombinant virus Bivalent Metavac®-RSV v.1, Bivalent Metavac®-RSV v.2 and Bivalent Metavac®-RSV v.3, illustrating that despite variable replicative kinetics, all of the three recombinant bivalent candidates lead to similar production yield.

TABLE 3
Recombinant bivalent viral loads

| | Mean viral | Mean viral |
|---|---|---|
| | titers before | titers after |
| | concentration | concentration |
| Recombinant viruses | (TCID50/ml) | (TCID50/ml) |
| Bivalent Metavac ®-RSV v.1 | 2.85E+05 | 1.90E+07 |
| Bivalent Metavac ®-RSV v.2 | 3.57E+05 | 3.20E+07 |
| Bivalent Metavac ®-RSV v.3 | 6.31E+05 | 2.55E+07 |

These results show the replicative capacities of the Bivalent Metavac®-RSV v.1, Bivalent Metavac®-RSV v.2 and Bivalent Metavac®-RSV v.3 viruses.

### Example 3: In Vitro Expression of Exogenous F RSV Proteins at the Surface of Cells Infected with the Recombinant Bivalent Metavac®-RSV Viruses

### Immunostaining Assay

LLC-MK2 cells were infected (t=0) with the Bivalent Metavac®-RSV v.1, Bivalent Metavac®-RSV v.2 and Bivalent Metavac®-RSV v.3 viruses, or with the wild-type recombinant rC-85473 HMPV strain at an MOI of 0.01. Hep-2 cells were infected (t=0) with RSV A2 strain at an MOI of 0.01. After 5 days of infection, infected cell monolayers were fixed in formaldehyde solution and specific immunostainings were performed with:
Monoclonal mouse antibody detecting the F HMPV protein (MAb HMPV24 BioRad MCA 4674);
Monoclonal humanized antibody detecting both pre-fusion and post-fusion forms of the F RSV protein (Palivizumab Synagis® AstraZeneca);
Monoclonal human antibody detecting the pre-fusion form of the F RSV protein (D25 Mab, Creative Biolabs).

Results are presented in FIG. 3.

Specific labelling is observed after peroxidase revelation and the representative images show that both HMPV F (after HMPV24 immunostaining) and RSV F (after Palivizumab immunostaining) fusion proteins are expressed and detected on cells infected with Bivalent Metavac®-RSV v.1, or Bivalent Metavac®-RSV v.2 and Bivalent Metavac®-RSV v.3 viruses.

As expected for controls, no immunostaining with MAb HMPV24 was observed on cells infected by RSV A2 strain (which does not express HMPV F fusion protein), and no immunostaining with Palivizumab Synagis® or D25 Mab was observed on cells infected by rC-85473 HMPV (which does not express RSV F fusion protein).

With monoclonal D25 immunostaining, the pre-fusion F RSV protein form was detected on cell infected with the Bivalent Metavac®-RSV v.2 and Bivalent Metavac®-RSV v.3 viruses with higher intensity than with the Bivalent Metavac®-RSV v.1, which argue in favour of stronger expression and exposition at cell surface of the stabilized pre-fusion F RSV protein, as expected.

### Flow Cytometry Assay

LLC-MK2 cells were infected (t=0) with the Bivalent Metavac®-RSV v.1, Bivalent Metavac®-RSV v.2 and Bivalent Metavac®-RSV v.3 viruses, or with the rC-85473 HMPV at an MOI of 0.1.

After 48 hours of infection, infected cell monolayers were trypsinized, resuspended and quantification of the F HMPV or F RSV protein expressions was performed in flow cytometry.

Infected cells were detected by GFP fluorescence and the detection of F HMPV and F RSV expression is performed with the following immunolabeling:
Monoclonal humanized antibody detecting both pre-fusion and post-fusion forms of the F RSV protein (Palivizumab Synagis® AstraZeneca) conjugated with R-Phycoerythrin (PE) fluorochrome;
Monoclonal mouse antibody detecting the F HMPV protein (MAb HMPV24 BioRad MCA 4674) conjugated with Alexa Fluor™647 (A647) fluorochrome.

Results are presented in table 4 below.

TABLE 4
Mean percentage expression of HMPV and hRSV antigens measured by flow cytometry
in cells infected by each Bivalent Metavac ® RSV vaccine candidate

| | Mean number of | Infected cells | Infected cells |
|---|---|---|---|
| | Infected cells | expressing HMPV-F | expressing RSV-F |
| | [%, GFP positive | [%, GFP and A647 | [%, GFP and PE |
| Virus | cells] | positive cells] | positive cells] |
| Bivalent Metavac ®-RSV v.1 | 14.75 | 94.75 | 65.5 |
| Bivalent Metavac ®-RSV v.2 | 15.95 | 94.85 | 54.6 |
| Bivalent Metavac ®-RSV v.3 | 20.6 | 85.2 | 47.2 |
| rC-85473 | 82.3 | 98.9 | NA |

NA: not applicable

The results reported in Table 4 show that more than 85% of the infected cells expose the F HMPV protein at their surface whereas 64.5%, 54.6% and 47.2% of the cells expose the F RSV proteins when they are infected with Bivalent Metavac®-RSV v.1, Bivalent Metavac®-RSV v.2 and Bivalent Metavac®-RSV v.3 viruses, respectively.

These results show the ability of the three Bivalent Metavac®-RSV v.1, Bivalent Metavac®-RSV v.2 and Bivalent Metavac®-RSV v.3 viruses to express and expose both the F RSV and the F HMPV proteins at the surface of infected cells, and in particular the stabilized pre-fusion F RSV form expressed by the Bivalent Metavac®-RSV v.2 and Bivalent Metavac®-RSV v.3 viruses.

### Example 4: In Vitro Expression of Exogenous F RSV Proteins at the Surface of the Bivalent Metavac®-RSV Particles

Viral suspensions of each Bivalent Metavac®-RSV viruses were prepared, filtered at 0.45 μm, concentrated by ultracentrifugation and then resuspended in NaCl. Viral suspensions were adsorbed on 200 Mesh coated Nickel grids and observed by transmission electron microscopy without labelling (FIG. 4A) or with co-immunolabeling using antibodies:

### Primary Antibodies:

Monoclonal humanized antibody detecting both pre-fusion and post-fusion forms of the F RSV protein (Palivizumab Synagis® AstraZeneca);
Polyclonal mouse serum detecting HMPV proteins (in house serum).

### Secondary Antibodies:

15 nm gold particle conjugated goat anti-human IgG detecting the anti-F RSV monoclonal humanized antibody;
5 nm gold particle conjugated goat anti-mouse IgG detecting the antibodies in the anti-HMPV mouse serum.

Results are presented in FIG. 4.

In FIG. 4A, the presence of typical pleiomorphic viral particles covered by transmembrane glycoproteins was observed for all the three Bivalent Metavac®-RSV v.1, Bivalent Metavac®-RSV v.2 and Bivalent Metavac®-RSV v.3 viruses.

In FIG. 4B, the F RSV proteins are detected at the surface of some viral particles for all the three Bivalent Metavac®-RSV v.1, Bivalent Metavac®-RSV v.2 and Bivalent Metavac®-RSV v.3 viruses, as revealed by immunolabeling with 15 nm gold particle (highlighted by black arrow). They are also immunolabelled by specific anti-HMPV serum (represented by 5 nm gold particles).

In FIG. 4C, viral stocks of each Bivalent Metavac®-RSV viruses were pre-incubated with specific anti-HMPV or specific anti-RSV neutralizing mouse sera before infection of LLC-MK2 cells. The % of specific seroneutralization was calculated by the measure of the intensity of the GFP fluorescence signal from infected cells. Neutralization of infection means that the glycoproteins at the surface of the viral particles, and more particularly the F proteins, are efficiently recognized by neutralizing antibodies present into the serum, that lead to the inhibition of virus attachment to cell receptors and consecutive viral infection. Bivalent Metavac®-RSV v.1, Bivalent Metavac®-RSV v.2 and Bivalent Metavac®-RSV v.3 viruses are highly neutralized by anti-HMPV serum and significantly neutralized by anti-RSV serum, in comparison with the wild-type hMPV rC-85473 strain. These results confirm the exposition of both F HMPV and F RSV proteins at the surface of Bivalent Metavac®-RSV v.1, v.2 and v.3 viral particles.

These results demonstrate the effective expression of both hMPV F protein and hRSV F protein at the surface of viral particles of the three Bivalent Metavac®-RSV v.1, Bivalent Metavac®-RSV v.2 and Bivalent Metavac®-RSV v.3 viruses.

### Example 5: Infection and Replicative Capacity of the Three Bivalent Metavac®-RSV v. 1, v.2 and v.3 Viruses in Ex Vivo 3D Reconstituted Human Respiratory Epithelium

3D reconstituted human respiratory epithelia (MucilAir® HAE, Epithelix) have been cultivated at the air-liquid interface following the supplier instructions.

Epithelia were then infected at an MOI 0.5 with the monovalent Metavac® or the bivalent Metavac®-RSV v.1, v.2 or v.3 viruses.

Representative pictures of viral propagation have been taken after 3, 5 and 7 days of infection with the monovalent Metavac®, bivalent Metavac®-RSV v.1, bivalent Metavac®-RSV v.2 and bivalent Metavac®-RSV v.3 viruses.

FIG. 5A shows extended GFP expression within human respiratory epithelia which reveals that the bivalent Metavac®-RSV v.1, v.2 and v.3 viruses are infectious and replicative, although their propagation into the tissue appear slower than the propagation of the monovalent Metavac®, considering the delay in GFP fluorescence detection.

In FIG. 5B, Trans-Epithelial Electric Resistance (TEER) measures were taken during the time-course of infections. The TEER measure corresponds to a relevant marker of epithelia integrity. Similarly to the monovalent Metavac®, limited variations of TEER were observed after infections by the bivalent Metavac®-RSV v.1, v.2 and v.3 viruses and correlate with an attenuated viral phenotype and the preservation of epithelia integrity.

In FIG. 5C, the viral genome quantification at the apical surface confirmed efficient production of viral progeny from infections by each of the bivalent Metavac®-RSV.

In FIG. 5D, immunolabeling with Palivizumab Synagis® reveal the expression of F RSV protein at the apical surface of 3D reconstituted human respiratory epithelium infected by the bivalent Metavac®-RSV v.1 virus.

These results show the capacity of the three different bivalent Metavac®-RSV v. 1, v.2 and v.3 viruses to infect and propagate into ex vivo human airway epithelial tissue, and further the ability of these bivalent Metavac®-RSV viruses to express the exogenous F RSV antigen at the surface of 3D reconstituted human respiratory epithelium.

### Example 6: In Vivo Characterization of the Recombinant Bivalent Metavac®-RSV v.1, v.2 and v. 3 Viruses on BALB/c Mice Viral Infection Models

BALB/c mice were infected by intranasal instillation with: 5×10⁵ TCID50 of recombinant bivalent Metavac®-RSV v.1, v.2 or v.3 viruses, on the basis of active immunizing dose determined for the monovalent Metavac® virus. The weight and the survival of the infected mice were monitored daily for 10 days (weight average over 5 mice±SEM).

In addition, 5 days after the infection, 2 mice per group underwent euthanasia for a measurement of pulmonary viral loads by RT-qPCR.

In FIG. 6A, no significant weight loss was observed during the time-course of the infections by the bivalent Metavac®-RSV v.1, v.2 or v.3 viruses, as expected for an attenuated viral strain.

FIG. 6B shows the results of viral gene quantification in lung of mice, 5 days post-infection. High level of both N HMPV and F RSV gene expression are observed, which confirm replicative property of the three bivalent Metavac®-RSV v.1, v.2 or v.3 viruses, and their capacity to express the F RSV gene in vivo.

These results show the capacity of three bivalent Metavac®-RSV viruses to infect and replicate in vivo, and to express the exogenous F RSV gene at a similar level to that of N HMPV gene into the pulmonary tissue of infected mice.

### Example 7: In Vivo Induction of Neutralizing Antibody Production after HMPV- or RSV-Prime Infection Followed by Boost Infection with the Bivalent Metavac®-RSV v.1, v.2 and v.3 Viruses on BALB/c Mice Model

BALB/c mice were infected by intranasal instillation with non-lethal doses of wild-type HMPV rC-85473 strain (1×10⁶ TCID50) or wild-type RSV A Long strain (1×10⁶ PFU), in order to induce a primary seroconversion of infected mice.

Three weeks after, HMPV- or RSV-primed BALB/c mice were infected by intranasal (boost) instillation with 5×10⁵ TCID50 of the recombinant bivalent Metavac®-RSV v.1, v.2 or v.3 viruses, on the basis of active immunizing dose determined for the monovalent Metavac® virus.

The weight and the survival of the mice was monitored daily for 10 days (weight average over 8 mice±SEM).

In addition, 5 days after the intranasal boost-infection, 3 mice per group underwent euthanasia to measure pulmonary viral loads by RT-qPCR.

In FIGS. 7D-7E, quantifications of N HMPV and F RSV genes by RT-qPCR indicate that pulmonary replication of the bivalent Metavac®-RSV v.1, v.2 or v.3 viruses is very weak in mice which are non-naïve for HMPV virus, whereas their pulmonary replication is high in mice which are non-naïve for RSV.

In FIGS. 7F-7G, the characterization of HMPV- or RSV-specific neutralizing antibody responses by seroneutralization assays show that the three bivalent Metavac®-RSV v.1, v.2 or v.3 viruses were able to induce a strong HMPV- or RSV-oriented antibody response depending on the initial non-naïve HMPV or RSV serological status

These results show the capacity of the three bivalent Metavac®-RSV v.1, v.2 or v. 3 viruses to induce in vivo a strong and specific neutralizing antibody response against HMPV and RSV viruses.

### Example 8: In Vivo Protective Properties of the Bivalent Metavac®-RSV v.3 Virus Against an Infectious Challenge with a Lethal Dose of the Wild Virus rC-85473 HMPV Strain

BALB/c mice were immunized twice, in 21-days interval, by intranasal instillation with 5×10⁵ TCID50 of the recombinant monovalent Metavac® or the bivalent Metavac®-RSV v.3 viruses, on the basis of active immunizing dose determined for the monovalent Metavac® virus, in comparison with a group of mice mock-immunized with culture medium (mock).

Twenty-one days after the boost-infection, mice endured a hMPV lethal viral challenge by intranasal instillation of 3×10⁶ TCID50 of the wild-type HMPV rC-85473 (an infectious dose resulting in more than 50% mortality rate for mice).

The weight and the survival of the mice was monitored daily for 14 days (weight average over 8 mice±SEM).

In FIG. 8B, 2-immunized mice showed no significant weight loss and complete survival after the lethal viral challenge whereas mock-immunized mice showed a very significant weight loss, resulting in the death of all mice by 8 days after the lethal challenge (FIG. 8C).

Five days after the viral challenge, 4 mice per group underwent euthanasia to measure pulmonary viral loads by RT-qPCR.

In FIG. 8D, both N HMPV and F RSV genes copies quantification indicated that the 2-doses immunization with the monovalent Metavac® or the bivalent Metavac®-RSV v.3 viruses were efficient to significantly restrain HMPV viral replication in lungs in comparison with mock-immunized mice.

Finally, in FIGS. 8E-8F, the quantification of HMPV- or RSV-specific neutralizing antibody responses were made from sera harvested prior to prime-intranasal and 21 days after the boost-instillation (except for mock-immunized group).

As expected, a high level of HMPV-neutralizing antibodies was measured in sera of mice immunized with 2 doses of monovalent Metavac® virus.

For mice double-immunized with the bivalent Metavac®-RSV v.3 virus, high neutralizing antibody titers against both HMPV and RSV viruses were detected 21 days after the boost-immunization and were persistent until 21 days after the HMPV viral challenge.

These results show the capacity of the bivalent Metavac®-RSV candidate (bivalent Metavac®-RSV v.3) to induce in vivo a strong specific neutralizing antibody response against both HMPV- and RSV viruses, and to fully protect mice challenged with a lethal dose of HMPV wild-type virus.

### Example 9: In Vivo Characterization of the Recombinant Bivalent Metavac®-RSV v. 1 and v. 3 Viruses on BALB/c Mice Viral Infection Models

BALB/c mice were infected by intranasal instillation with 5×10⁵ TCID50 of recombinant bivalent Metavac®-RSV v.1 or v.3 viruses, on the basis of active immunizing dose determined for the monovalent Metavac® virus.

FIG. 9A shows the results of viral gene quantification in broncho-alveolar lavages of mice, 5 days post-infection. High level of both N HMPV and F RSV gene expression are observed, which confirm the presence and the replicative property of the bivalent Metavac®-RSV v. 1 and v.3 viruses in lower respiratory tracts of infected animals and their capacity to express the F RSV gene in vivo.

In addition, 5 days after the infection, 3 mice per group underwent euthanasia for a lung tissue harvest. Complete lungs were fixed with formaldehyde solution for further histopathological analysis.

In FIG. 9B, a low inflammatory profile was described after infection by the bivalent Metavac®-RSV v.1 or v.3 viruses, at the image of the low inflammation score measured after infection with the monovalent LAV candidate Metavac® and in contrast with the high pro-inflammatory score measured after HMPV WT infection.

Overall, these results highlight the capacity of both bivalent Metavac®-RSV viruses to infect and replicate in vivo, to express the exogenous F RSV gene, and to induce a low inflammatory response into the pulmonary tissue of infected mice, as expected from live-attenuated vaccine candidates.

### Example 10: In Vivo Protective Properties of the Bivalent Metavac®-RSV v.1 and v.3 Viruses Against an Infectious Challenge with a Lethal Dose of the Wild Virus rC-85473 HMPV Strain

BALB/c mice were immunized twice, in 21-days interval, by intranasal instillation with 5×10⁵ TCID50 of the recombinant monovalent Metavac® or bivalent Metavac®-RSV v.1 or v.3 viruses, on the basis of active immunizing dose determined for the monovalent Metavac® virus, in comparison with a group of mice mock-immunized with culture medium (mock) or a group of mice immunized via intramuscular route with an adjuvanted split of HMPV WT virus (HMPV split), as surrogate of a vaccination with HMPV protein vaccine.

Twenty-one days after the boost-infection, mice endured a HMPV lethal viral challenge by intranasal instillation of 2×10⁶ TCID50 of the wild-type HMPV rC-85473 (an infectious dose resulting in more than 50% mortality rate for mice).

The weight and the survival of the mice was monitored daily for 14 days (weight average over 8 mice±SEM).

In FIGS. 10A and 10B, immunized mice (vaccination with recombinant monovalent Metavac® or bivalent Metavac®-RSV v.1 or v.3 viruses or HMPV split) show a significant weight loss reduction and complete survival after the lethal viral challenge whereas mock-immunized mice showed a very significant weight loss, resulting in the death of all mice by 6 days after the lethal challenge (FIG. 10B).

Five days after the viral challenge, 3 mice per group underwent euthanasia for a lung tissue harvest for further histopathological analysis and 2 or 4 mice per group underwent euthanasia to measure pulmonary viral loads by RT-qPCR.

In FIG. 10C, different inflammatory profiles are described, depending of the vaccine candidate and route of administration. After lethal HMPV challenge, reduced interstitial, peribronchial, intra-alveolar and pleural inflammations seem to be induced in mice vaccinated with the bivalent Metavac®-RSV v.1 or v.3 viruses, at the difference of the high inflammation score measured induced after immunization with HMPV split, corresponding to an enhanced disease syndrome as described in literature after immunization with inactive vaccine.

In FIG. 10D, both N HMPV and F RSV genes copies quantification indicates that the 2-doses immunization with the monovalent Metavac® or the bivalent Metavac®-RSV v.1 and v.3 viruses were efficient to significantly restrain HMPV viral replication in lungs in comparison with mock-immunized mice and with HMPV split-immunized mice.

In FIG. 10E, the quantification of homologous or heterologous HMPV-specific neutralizing antibody responses were made from sera harvested along the time-course of the protocol. As expected, a high level of HMPV-neutralizing antibodies was measured in sera of mice immunized with 2 doses of monovalent Metavac® virus. For mice double-immunized with the bivalent Metavac®-RSV v.1 and v.3 viruses, similar high neutralizing antibody titers against both HMPV A and B viruses were detected 21 days after the boost-immunization, and were persistent or augmented until 21 days after the HMPV viral challenge.

Finally, in FIG. 10F, the quantification of HMPV-specific IgG antibodies was made from sera harvested along the time-course of the protocol. As expected, given neutralizing antibody titers, a high level of IgG antibodies was measured in sera of mice immunized with 2 doses of monovalent Metavac® or bivalent Metavac®-RSV v.1 and v.3 viruses. At the difference, immunization with adjuvanted HMPV split seems to induced higher level of neutralizing and IgG antibodies as soon as 21 days after the second immunization but the antibody responses appear to be decreasing after the HMPV challenge.

These results show the capacity of the bivalent Metavac®-RSV candidates (both Metavac®-RSV v.1 and v.3) to induce in vivo a strong specific neutralizing antibody response against both HMPV A and B viruses, and to fully protect mice challenged with a lethal dose of HMPV wild-type virus.

### Example 11: In Vivo Protective Properties of the Bivalent Metavac®-RSV v.1 and v.3 Viruses Against an Infectious Challenge with a Recombinant RSV-Luc WT Virus, Expressing a Luciferase Protein

BALB/c mice were immunized twice, in 21-days interval, by intranasal instillation with 5×10⁵ TCID50 of the bivalent Metavac®-RSV v.1 or v.3 viruses, on the basis of active immunizing dose determined for the monovalent Metavac® virus, in comparison with a group of mice mock-immunized with culture medium (mock) or a group of mice immunized by intranasal instillation with 5×10⁵ PFU of a recombinant RSV WT virus (RSV-mCh).

Twenty-one days after the boost-infection, mice endured a RSV infectious challenge by intranasal instillation of 1×10⁵ PFU of rRSV-Luc virus, a recombinant RSV A WT virus expressing a luminescent luciferase protein in vivo (Rameix-Welti et al., 2014).

The weight of animals was monitored daily for 14 days with no weight loss, as expected from RSV infection in mouse model.

In FIGS. 11A and 11B, immunized mice (vaccination with bivalent Metavac®-RSV v. 1 or v.3 viruses or RSV WT) show a significant luciferase activity and bioluminescence reduction in nose and lungs after the viral challenge, whereas mock-immunized mice show a high intensity of luminescence, especially in lungs 5 days post-challenge, as result of RSV viral replication in pulmonary tissue.

Four days after the viral challenge, 4 mice per group underwent euthanasia to measure pulmonary viral loads by RT-qPCR.

In FIG. 11C, in accordance with precedent luminescence results, F RSV genes copies quantification indicates that the 2-doses immunization with the bivalent Metavac®-RSV v. 1 and v.3 viruses tend to reduce and restrain RSV viral replication in lungs, in comparison with mock-immunized mice, and that no residual N HMPV gene material is detected in lungs of mice more than 3 weeks after the boost-immunization with bivalent Metavac®-RSV candidates.

In FIG. 11D, the quantification of homologous or heterologous RSV-specific neutralizing antibody responses was made from sera harvested along the time-course of the protocol.

As expected, an induction of RSV-neutralizing antibodies was measured in sera of mice immunized with 2 doses of bivalent Metavac®-RSV v.1 and RSV WT viruses, and higher levels of neutralizing antibodies specific to RSV A and B strains seem to be induced by the vaccination with the bivalent Metavac®-RSV v.3 candidate 21 days after the RSV viral challenge.

These results show the capacity of the bivalent Metavac®-RSV candidates (bivalent Metavac®-RSV v.1 and v.3) to induce in vivo a strong specific neutralizing antibody response against both RSV A and B viruses, and to restrain RSV replication in upper and lower respiratory tract of mice challenged with a RSV virus.

### REFERENCES

### Patents

WO 2020/021180
WO 2020/120910
EP 3 868 874

### BIBLIOGRAPHIC REFERENCES

van den Hoogen B G, of Jong J C, Groen J, Kuiken T, of Groot R, Fouchier R A, et al. A newly discovered human pneumovirus isolated from young children with respiratory tract disease. Nat Med. 2001; 7 (6): 719-24
Peret T C, Boivin G, Li Y, Couillard M, Humphrey C, Osterhaus A D, Erdman D D, Anderson L J. Characterization of human metapneumoviruses isolated from patients in North America. J Infect Dis. 2002 Jun. 1; 185 (11): 1660-3.
Rostad C A, Stobart C C, Gilbert B E, Pickles R J, Hotard A L, Meng J, Blanco J C G, Moin S M, Graham B S, Piedra P A, Moore M L. A Recombinant Respiratory Syncytial Virus Vaccine Candidate Attenuated by a Low-Fusion F Protein Is Immunogenic and Protective against Challenge in Cotton Rats. J Virol. 2016 Jul. 27; 90 (16): 7508-7518.
Stobart C C, Rostad C A, Ke Z, Dillard R S, Hampton C M, Strauss J D, Yi H, Hotard A L, Meng J, Pickles R J, Sakamoto K, Lee S, Currier M G, Moin S M, Graham B S, Boukhvalova M S, Gilbert B E, Blanco J C, Piedra P A, Wright E R, Moore M L. A live RSV vaccine with engineered thermostability is immunogenic in cotton rats despite high attenuation. Nat Commun. 2016 Dec. 21; 7:13916.
Karron R A, Luongo C, Mateo J S, Wanionek K, Collins P L, Buchholz U J. Safety and Immunogenicity of the Respiratory Syncytial Virus Vaccine RSV/ΔNS2/Δ1313/11314L in RSV-Seronegative Children. J Infect Dis. 2020 Jun. 16; 222 (1): 82-91.
Mclellan J S, Chen M, Joyce M G, Sastry M, Stewart-Jones G B, Yang Y, Zhang B, Chen L, Srivatsan S, Zheng A, Zhou T, Graepel K W, Kumar A, Moin S, Boyington J C, Chuang G Y, Soto C, Baxa U, Bakker A Q, Spits H, Beaumont T, Zheng Z, Xia N, Ko S Y, Todd J P, Rao S, Graham B S, Kwong P D. Structure-based design of a fusion glycoprotein vaccine for respiratory syncytial virus. Science. 2013 Nov. 1; 342 (6158): 592-8.
Needleman S B, Wunsch C D. A general method applicable to the search for similarities in the amino acid sequence of two proteins. J Mol Biol. 1970 March; 48 (3): 443-53. doi: 10.1016/0022-2836 (70) 90057-4. PMID: 5420325.
Hamelin M E, Gagnon C, Prince G A, Kiener P, Suzich J, Ulbrandt N, Boivin G. Prophylactic and therapeutic benefits of a monoclonal antibody against the fusion protein of human metapneumovirus in a mouse model. Antiviral Res. 2010 October; 88 (1): 31-7.
Dubois J, Cavanagh M H, Terrier O, Hamelin M E, Lina B, Shi R, et al. Mutations in the fusion protein heptad repeat domains of human metapneumovirus impact on the formation of syncytia. The Journal of general virology. 2017; 98 (6): 1174-80.
Lê V B, Dubois J, Couture C, Cavanagh M H, Uyar O, et al. Human metapneumovirus activates NOD-like receptor protein 3 inflammasome via its small hydrophobic protein which plays a detrimental role during infection in mice. (2019) PLOS Pathogens 15 (4): e1007689. https://doi.org/10.1371/journal.ppat.1007689
Dubois J, Pizzorno A, Cavanagh M-H, Padey B, Nicolas de Lamballerie C, Uyar O, Venable M-C, Carbonneau J, Traversier A, Julien T, Lavigne S, Couture C, Lina B, Hamelin M-È, Terrier O, Rosa-Calatrava M, Boivin G. Strain-Dependent Impact of G and S H Deletions Provide New Insights for Live-Attenuated HMPV Vaccine Development. Vaccines. 2019; 7 (4): 164. https://doi.org/10.3390/vaccines7040164
Rameix-Welti, M A., Le Goffic, R., Hervé, P L. et al. Visualizing the replication of respiratory syncytial virus in cells and in living mice. Nat Commun 5, 5104 (2014). https://doi.org/10.1038/ncomms6104

1-15. (canceled)

16. A viral strain derived from the human metapneumovirus (hMPV) strain having the genome sequence represented in SEQ ID NO. 1, wherein said genome sequence comprises the following genetic modifications:
(i) inactivation of the endogenous gene coding for the SH protein and/or for the G protein, and
(ii) presence of an exogenous nucleotide sequence comprising a sequence coding for at least one extracellular domain of the F protein of the human respiratory syncytial virus (hRSV), said domain being wild-type or mutated.

17. The viral strain according to claim 16, wherein the endogenous gene coding for the SH protein is deleted.

18. The viral strain according to claim 16, wherein the exogenous nucleotide sequence comprises a sequence coding for the wild-type extracellular domain of the F protein of hRSV.

19. The viral strain according to claim 18, wherein the exogenous nucleotide sequence consists in a sequence coding for the three wild-type domains (cytoplasmic, transmembrane and extracellular) of the F protein of hRSV.

20. The viral strain according to claim 16, wherein the exogenous nucleotide sequence comprises a sequence coding for a mutated domain of the F protein of hRSV.

21. The viral strain according to claim 20, wherein the exogenous nucleotide sequence consists in a sequence coding for the wild-type cytoplasmic domain, the wild-type transmembrane domain, and the mutated extracellular domain corresponding to the stabilized prefusion state of the F protein of hRSV.

22. The viral strain according to claim 16, wherein the exogenous nucleotide sequence comprises:
a. a sequence originating from hRSV, coding for at least the extracellular domain of the F protein, said domain being wild-type or mutated; and
b. a sequence originating from hMPV, coding for at least one domain of the F protein, said domain being chosen among the cytoplasmic domain and the transmembrane domain, said domain being wild-type or mutated,
said exogenous sequence encoding a chimeric hRSV/hMPV F protein.

23. The viral strain according to claim 22, wherein the exogenous nucleotide sequence consists in:
a. a sequence originating from hRSV, coding for a mutated extracellular domain corresponding to the stabilized prefusion state of the F protein of hRSV; and
b. a sequence originating from hMPV, coding for the wild-type cytoplasmic domain and the wild-type transmembrane domain of the F protein of hMPV.

24. The viral strain according to claim 22, wherein the exogenous nucleotide sequence consists in:
a. a sequence originating from hRSV, coding for a wild-type extracellular domain of the F protein of hRSV; and
b. a sequence originating from hMPV, coding for the wild-type cytoplasmic domain and the wild-type transmembrane domain of the F protein of hMPV.

25. A genetic cassette comprising at least one of the following nucleotide sequences:
a sequence coding for a mutated extracellular domain corresponding to the stabilized prefusion state of the F protein of hRSV, and a sequence coding for the wild-type cytoplasmic domain and the wild-type transmembrane domain of the F protein of hMPV, and
a sequence coding for a wild-type extracellular domain of the protein of hRSV, and a sequence coding for the wild-type cytoplasmic domain and the wild-type transmembrane domain of the F protein of hMPV.

26. A viral strain derived from a human metapneumovirus (hMPV) strain comprising in its genome a genetic cassette according to claim 25.

27. A method for preventing and/or treating infections by at least one respiratory virus, comprising the administration of a viral strain according to claim 16 to individuals infected or susceptible to be infected by said respiratory virus.

28. The method for preventing and/or treating infections according to claim 27, wherein the at least one respiratory virus is from the Pneumoviridae family.

29. A vaccine composition comprising, in a pharmaceutically acceptable vehicle, at least one viral strain according to claim 16, and optionally an adjuvant.

30. The viral strain according to claim 17, wherein the exogenous nucleotide sequence comprises a sequence coding for the wild-type extracellular domain of the F protein of hRSV.

31. The viral strain according to claim 17, wherein the exogenous nucleotide sequence comprises a sequence coding for a mutated domain of the F protein of hRSV.

32. The viral strain according to claim 22, wherein the exogenous nucleotide sequence comprises a sequence coding for a mutated extracellular domain of the F protein, corresponding to the stabilized prefusion state of F protein.

33. The viral strain according to claim 32, wherein the exogenous nucleotide sequence comprises a sequence coding for a mutated extracellular domain of the F protein corresponding to the stabilized prefusion state of F protein.

34. The viral strain according to claim 32, wherein the exogenous nucleotide sequence consists in a sequence coding for the wild-type cytoplasmic domain, the wild-type transmembrane domain, and the mutated extracellular domain corresponding to the stabilized prefusion state of the F protein of hRSV.

35. The viral strain according to claim 17, wherein the exogenous nucleotide sequence comprises:
a. a sequence originating from hRSV, coding for at least the extracellular domain of the F protein, said domain being wild-type or mutated; and
b. a sequence originating from hMPV, coding for at least one domain of the F protein, said domain being chosen among the cytoplasmic domain and the transmembrane domain, said domain being wild-type or mutated,
said exogenous sequence encoding a chimeric hRSV/hMPV F protein.

36. The genetic cassette according to claim 25, comprising at least one of the following nucleotide sequences:
a sequence as shown in SEQ ID NO. 8; and
a sequence as shown in SEQ ID NO. 9.

37. The viral strain according to claim 26, that is attenuated.

38. The method for preventing and/or treating infections by at least one respiratory virus from the Pneumoviridae family according to claim 28, wherein said respiratory virus is a human metapneumovirus (hMPV) and/or the human syncytial respiratory virus (hRSV).

39. A method for preventing and/or treating infections by at least one respiratory virus, comprising the administration of a viral strain according to claim 17 to individuals infected or susceptible to be infected by said respiratory virus.

40. The method for preventing and/or treating infections according to claim 39, wherein the at least one respiratory virus is a human metapneumovirus (hMPV) and/or the human syncytial respiratory virus (hRSV).

41. A vaccine composition comprising, in a pharmaceutically acceptable vehicle, at least one viral strain according to claim 17, and optionally an adjuvant.


SEQUENCE LISTING (13 sequences)

SEQ ID NO: 1 - DNA, 13394 bp, Human metapneumovirus
  source 1..13394: mol_type = genomic DNA
gcgaaaaaaacgcgtataaattagattacaaaaaaatatgggacaagtgaaaatgtctcttcaagggattcacctgagtgatctatcatacaagcatgctatattaaaagagtctcagtacacaataaaaagagatgtgggtacaacaactgcagtgacaccctcatcattgcaacaagaaataacgctgttgtgtggagaaattctgtatgctaaacatgctgattacaaatatgctgcagaaataggaatacaatatattagcacagctttaggatcagagagagtgcagcagattctgaggaactcaggcagtgaagtccaagtggtcttaaccagaacgtactctctggggaaagttaaaaacaataaaggagaagatttacagatgttagacatacacggggtagagaagagctgggtagaagagatagacaaagaagcaaggaaaacaatggcaaccttgcttaaggaatcatcaggtaatatcccacaaaatcagaggccctcagcaccagacacacccataatcttattatgtgtaggtgcattaatatttactaagctagcatcaaccatagaagtgggactagagaccacagtcagaagggctaaccgtgtactaagtgatgcactcaagagataccctagaatggacatcccaaaaattgccagatccttctatgacttatttgaacaaaaagtgtatcacagaagtttgttcattgagtatggcaaagcattaggctcatcatctacaggcagcaaagcagaaagtctatttgttaatatattcatgcaagcttatggagccggtcaaacaatgctaaggtggggggtcattgccaggtcatccaacaatataatgttaggacatgtatctgtccaagctgagttaaaacaggtcacagaagtctatgacttggtgcgagaaatgggccctgaatctggacttctacatttaaggcaaagcccaaaagctggactgttatcactagccaactgtcccaactttgcaagtgttgttctcggaaatgcctcaggcttaggcataatcggtatgtatcgtgggagagtaccaaacacagaattattttcagcagcagaaagttatgccaaaagtttgaaagagagcaataaaatcaatttctcttcattaggacttacagatgaagagaaagaggctgcagaacatttcttaaatgtgagtgacgacagtcaaaatgattatgagtaattaaaaaagtgggacaagtcaaaatgtctttccctgaaggaaaagatattcttttcatgggtaatgaagcagcaaaattagcagaagctttccagaaatcattaaggaaaccaagtcataaaagatctcaatctattataggagaaaaagtgaacactgtatcagaaacattggaattacctactatcagtagacctgcaaaaccaaccatactgtcagaaccaaagttagcatggactgataaaggtggggcaatcaaaactgaaataaagcaagcaatcaaagtcatggatcctattgaggaagaagagtctactgagaagaaggtgctgccctccagtgatgggaaaacccctgcagaaaagaaactgaaaccatcaactaacaccaaaaagaaagtttcgtttacaccaaatgaaccaggaaaatatacaaagttggaaaaagatgctctagatttgctctcagataatgaagaagaagatgcagaatcttcaatcttaacctttgaagaaagagatacttcatcgttaagcattgaggccagattggaatcaatagaggagaaattaagcatgatattagggctattaagaacactcaacattgctacagcaggacccacagcagcaagagatgggatcagagatgcaatgattggcgtaagagaggaattaatagcagacataataaaggaagccaaagggaaagcagcagaaatgatggaagaggaaatgagtcaacgatcaaaaataggaaacggtagtgtaaaactaacagagaaagcaaaagagcttaacaaaattgttgaagatgaaagcacaagtggagaatctgaagaagaagaagaaccaaaagacatacaagacaatagtcaagaagatgacatttaccagttaattatgtagtttaataaaaataaacaatgggacaagtaaaaatggagtcctacctagtagacacttatcaaggcattccttacacagcagctgttcaagttgatctaatagaaaaggacctgttacctgcaagcctaacaatatggttccctttgtttcaggccaacacaccaccagcagtgctgctcgatcagttgaaaaccctaacaataaccactctgtatgctgcatcacaaaatggtccaatactcaaagtgaatgcatcagcccaaggtgcagcaatgtctgtacttcccaaaaaatttgaagtcaatgcgactgtagcactcgatgaatatagcaaattggaatttgacaaactcacagtctgtgaagtaaaaacagtttacttaacaaccatgaaaccatacgggatggtatcaaaatttgtgagctcagccaaatcagttggcaaaaaaacacatgatctaatcgcactgtgtgattttatggatctagaaaagaacacacctgttacaataccagcattcatcaaatcagtttcaatcaaagagagtgagtcagctactgttgaagctgctataagcagtgaagcagaccaagctctaacacaggccaaaattgcaccttatgcgggattgattatgatcatgactatgaacaatcccaaaggcatattcaaaaagcttggagctgggactcaagttatagtagaactaggagcatatgtccaggctgaaagcataagtaaaatatgcaagacttggagccatcaagggacaagatatgtgttgaagtccagataacagccaagcaccttggccaagagctactaactctatctcatagattataaagtcaccattctagttatataaaaatcaagttagaacaagaattaaatcaatcaagaatgggacaaataaaaatgtcttggaaagtggtgatcattttttcattgttaataacacctcaacacggtcttaaagagagctatttagaagagtcatgtagcactataactgaaggatatctcagtgttctgaggacaggttggtataccaacgtttttacactggaggtaggtgatgtagagaaccttacatgtgctgatggacctagcttaataaaaacagaattagacctgaccaaaagtgcactaagagaactcagaacagtttctgctgatcaactggcaagagaggagcaaattgagaatcccagacaatctagatttgttctaggagcaatagcactcggtgttgcaacagcagctgcagttacagcaggtgttgcaattgccaaaaccatccggcttgaaagtgaagtaacagcaattaagaatgccctcaaaaagaccaatgaagcagtatctacattggggaatggagttcgagtgttggcaactgcagtgagggagctggaagattttgtgagcaagaatctaacacgtgcaatcaacaaaaacaagtgcgacattgctgacctgaaaatggccgttagcttcagtcaattcaacagaaggtttctaaatgttgtgcggcaattttcagacaatgctggaataacaccagcaatatccttggacttaatgacagatgctgaactagccagagctgtttccaacatgccaacatctgcaggacaaataaaactgatgttggagaaccgtgcaatggtaagaagaaaggggttcggaatcctgataggagtttacggaagctccgtaatttacatggtgcaactgccaatctttggagttatagacacgccttgctggatagtaaaagcggccccttcttgctcagaaaaaaagggaaactatgcttgccttttaagagaagatcaaggatggtattgtcagaatgcagggtcaactgtttactacccaaatgaaaaagactgcgaaacaagaggagaccatgtcttttgcgacacagcagcaggaatcaatgttgctgagcagtcaaaggagtgcaacatcaacatatccactactaattacccatgcaaagttagcacaggaagacaccctatcagtatggttgcactgtctcctcttggggctttggttgcttgctacaagggagtgagctgttccattggcagcaacagagtagggatcatcaagcaactgaacaaaggctgctcttatataaccaaccaagacgcagacacagtgacaatagacaacactgtataccagctaagcaaagttgagggcgaacagcatgttataaaaggaaggccagtgtcaagcagctttgatccagtcaaatttcctgaagatcaattcaatgttgcacttgaccaagttttcgaaagcattgagaacagtcaggccttggtggatcaatcaaacagaatcctaagcagtgcagagaaaggaaacactggcttcatcattgtaataattctaattgctgtccttggctctaccatgatcctagtgagtgtttttatcataataaagaaaacaaagaaacccacaggagcacctccagagctgagtggtgtcacaaacaatggcttcataccacataattagttaattaaaaataaagtaaattaaattaaaataaaataaaattaaaattaaaataaaataaaaataaaaatttgggacaaatcataatgtctcgcaaggctccatgcaaatatgaagtgcggggcaaatgcaatagaggaagtgagtgcaagtttaaccacaattactggagttggccagatagatacttactaataagatcaaattatttattaaatcaacttttaaggaacactgatagagctgatggcttatcaataatatcaggagcaggcagagaagataggacacaagattttgtcctaggttccaccaatgtggttcaaggttatattgatgataaccaaagcataacaaaagctgcagcctgttacagtctacataatataatcaaacaactacaagaagttgaagttaggcaggctagagataacaaaccatctgacagcaaacatgtggcacttcacaacttagtcctatcttatatggagatgagcaaaattcctgcatctttaatcaacaatctcaaaagactgccgagagagaaactgaaaaaattagcaaagcttataattgacttatcagcaggtgctgaaaatgactcttcatatgccttgcaagacagtgaaagcactaatcaagtgcagtgagcatggtcctgttttcattactatagaggttgattacatgatatggactcataaggacttaaaagaagctttatctaatgggatagtgaagtctcatactaacatttacaattgttatttagaaaacatagaaattatatatgtcaaggcttacttaagttagtaaaaacacatcagagtgggataaatgacaatgataacattagatgtcattaaaagtgatgggtcttcaaaaacatgtactcacctcaaaaaattaattaaagaccactctggtaaagtgcttattgtacttaagttaatattagctttactaacatttctcacagtgacaatcaccatcaattatataaaagtagaaaacaatctgcaaatatgtcagtcaaaaactgaatcagacaaaaaggactcatcatcaaataccacatcagtcacaaccaagactactctaaatcatgatataacacagtattttaaaagtttgattcaaaggtatacaaactctgcaataaacagagacacatgctggaaaataagcagaaatcaatgcacaaacataacaacatacaaatttttatgttttaaatctgaagaaacaaaaaccaacaattgtgataaactgacagatttatgcagaaacaaaccaaaaccagctgttgaagtgtatcacatagtagaatgccattgtatatacacagttaaatggaagtgctatcattacccaatagatgaaacccaatcctaaataacactagattaggatccatccaagtctgttagttcaacaatttagttatttaaaaatattttgaaaacaagtaagtttctatgatacttcataataataagtaataattaattgcttaatcatcatcacaacattattcgaaaccataactattcaatttaagaagtaaaaacaataatatgggacaagtagttatggaggtgaaagtggagaacattcgaacaatagatatgctcaaagcaagagtgaaaaatcgtgtggcacgcagcaaatgctttaaaaatgcctctttgatcctaataggaataactacattgagtatagccctcaatatctatctaatcataaactatacaatgcaagaaaacacatccgaatcagaacatcacaccagctcatcacccatggaatccagcagggaaactccaacagtccctatggacaactcagacaccaatccaggctcacagtatccaactcaacagtccacagaaggctccacactctactttgcagcctcagcaagctcaccagagacagaaccaacatcaacaccagacacaacaagccgcccgcccttcgtcgacacacacacaacactaccaagtgcaagcagaacaaggacaagtccggcagtccacacaaaaaacaatccaaggacaagccccagaacacattccccaccatgggcaatgacaaggacggtccgtggaaccaccactctccgcacaagcagcacaagaaaaagaccgtccacagcatcagtccaacctgacagcagcgcaacaacccacaaacacgaagaagcaagcccagtgagcccgcaaacatctgcgagcacagcaagaccacaaaggaagggcatggaggccagcacatcaacaacatacaaccaaactagttaacaaaaaatacaaaataactctaagataaaccatatagacaccaacaattgagaagccaaaaggcaattcacaatctctccaaaaaggcaacaacaccatattagctccgcttaaatctccctggaaaaaacactcgcccatataccaactataccacaaccatcccaagaaaaaaagctgggtaaaacaacacccaagagacaaataacaatggatcctcttaatgaatccactgttaatgtctatcttcccgactcatatcttaaaggagtgatttcttttagtgagactaatgcaattggttcatgtctcttaaaaagaccttacctaaaaaatgacaacactgcaaaagttgccatagagaatcctgttatcgagcatgttagactcaaaaatgcaatcaattctaagatgaaaatatcagattacaggatagtagagccagtaaacatgcaacatgaaattatgaagaatgtacacagttgtgagctcacattattaaaacagtttttaacaaggagtaaaaatattagcactcttaaattaaatatgatatgtgattggctgcagttaaagtctacatcagatgatacctcaatcttaagttttatagatgtagaatttatacctagctgggtaagcaattggtttagtaattggtacaatctcaacaagttgattctggaattcaggaaagaagaagtaataagaactggttcaatcttgtgtaggtcattgggtaaattagtttttgttgtatcatcatacggatgtatagtcaagagcaacaaaagcaaaagagtgagcttcttcacatacaatcaactgttaacatggaaagatgtgatgttaagtagattcaatgcaaatttctgtatatgggtaagcaacagtctgaatgaaaatcaagaagggttagggttgagaagtaatctgcaaggcatattaactaataagctatatgaaactgtagattatatgcttagtttgtgttgcaatgaaggtttctcacttgtgaaagagtttgagggttttattatgagtgaaatccttaggattactgaacatgctcaattcagtactagatttagaaatactttattaaatggattaactgatcaattgacaaaattaaaaaataaaaacagactcagagttcatggtaccgtgttagaaaataatgattatccaatgtatgaagttgtacttaaattattaggagatactttgagatgtattaaattattaatcaataaaaacttagagaatgctgctgaattatactatatatttagaatattcggtcacccaatggtagatgaaagagatgcaatggatgctgtcaaattaaacaatgaaatcacaaaaatcctcaggttggagagcttgacagaactaagaggggcattcatattaaggattatcaaaggatttgtagacaacaacaaaagatggccgaaaattaaaaacttaaaagtgcttagtaaaagatggactatgtacttcaaagcaaaaagttaccctagtcaacttgaattaagtgaacaagattttttagagcttgctgcaatacagtttgaacaagagttttctgttcctgaaaaaaccaaccttgagatggtattaaatgataaagctatatcacctcctaaaagattaatttggtctgtgtacccaaaaaattacttacctgagacaataaaaaatcgatatctagaagagactttcaatgcaagtgatagtctcaaaacaagaagagtactagagtactatttgaaagataataaattcgaccaaaaagaacttaaaagttatgtggttaaacaagaatatttaaatgataaggatcatattgtctcgctaactggaaaagaaagagaattaagtgtaggtagaatgtttgctatgcaaccaggaaaacagcgacaaatacaaatattggctgaaaaattgttagctgataatattgtaccctttttcccagaaactttaacaaagtatggtgatctagatcttcagagaataatggaaatcaaatcagaactttcttctattaaaaccagaagaaatgatagttataataattacattgcaagagcatccatagtaacagatttaagtaagttcaaccaagcctttaggtatgaaactacagcgatctgtgcggatgtagcagatgaactacatggaacacaaagcctattctgttggttacatcttatcgttcctatgactacaatgatatgtgcctatagacatgcaccaccagaaacaaaaggtgaatatgatatagataagatagaagagcaaagtggtttatatagatatcatatgggtggtattgaaggatggtgtcaaaaactctggacaatggaagctatatctttattagatgttgtatctgtaaagacacgatgtcaaatgacatctttattaaacggtgacaaccaatcaatagatgtaagtaaaccagttaagttatctgagggtttagatgaagtgaaagcagattatagcttggctgtaaaaatgctaaaagaaataagagatgcatacagaaatataggccataaacttaaagaaggggaaacatatatatcaagagatcttcagtttataagtaaggtgattcaatctgaaggagtaatgcatcctacccctataaaaaagatcttaagagtgggaccatggataaacacaatattagatgacattaaaaccagtgcagagtcaatagggagtctatgtcaggaattagaatttaggggggaaagcataatagttagtctgatattaaggaatttttggctgtataatttatacatgcatgaatcaaagcaacaccccctagcagggaagcagttattcaaacaactaaataaaacattaacatcagtgcagagattttttgaaattaaaaaggaaaatgaagtagtagatctatggatgaacataccaatgcagtttggaggaggagatccagtagtcttctatagatctttctatagaaggacccctgattttttaactgaagcaatcagtcatgtagatattctgttaaaaatatcagccaacataagaaatgaagcgaaaataagtttcttcaaagccttactgtcaatagaaaaaaatgaacgtgctacactgacaacactaatgagagaccctcaagctgtgggctcagagcgacaagcaaaagtaacaagtgatatcaatagaacagcagttaccagcatcttaagtctttctccaaatcaacttttcagcgatagtgctatacactacagtagaaatgaagaagaggtcggaatcattgctgacaacataacacctgtttatcctcatggactgagagttttgtatgaatcattaccttttcataaagctgaaaaagttgtaaatatgatatcaggaacaaaatccataaccaacttattacagagaacatctgctattaatggtgaagatattgacagagctgtatccatgatgctggagaacctaggattattatctagaatattgtcagtagttgttgatagtatagaaattccaaccaaatctaatggtaggctgatatgttgtcagatatctagaaccctaagggagacatcatggaataatatggaaatagttggagtaacatcccctagcatcactacatgcatggatgtcatatatgcaactagctctcatttgaaagggataatcattgaaaagttcagcactgacagaactacaagaggtcaaagaggtccaaagagcccttgggtaggatcgagcacgcaagagaaaaaattagttcctgtttataacagacaaattctttcaaaacaacaaagagaacagctagaagcaattggaaaaatgagatgggtatataaagggacaccaggtttaagacgattactcaataagatttgtcttggaagtttaggcattagttacaaatgtgtaaaacctttattacctaggtttatgagtgtaaatttcctacacaggttatctgtcagtagtagacctatggaattcccagcatcagttccagcttatagaacaacaaattaccattttgacactagtcctattaatcaagcactaagtgaaagatttgggaatgaagatattaatttggtcttccaaaatgcaatcagctgtggaattagcataatgagtgtagtagaacaattaactggtaggagtccaaaacagttagttttaataccccaattagaagaaatagacattatgccaccaccagtgtttcaagggaaattcaattataagctagtagataagataacttctgaccaacatatcttcagtccagacaaaatagatatgttaacactggggaaaatgctcatgccaactataaaaggtcagaaaacagatcagttcttgaacaagagagagaattatttccatgggaacaatcttattgagtctttgtcagcagcgttagcatgtcattggtgtgggatattaacagagcaatgtatagaaaataatattttcaagaaagactggggtgacgggttcatatcggatcatgcttttatggacttcaaaatattcctatgtgtctttaaaactaaacttttatgtagttggggatcccaagggaaaaacattaaagatgaagatatagtagatgaatcgatagataaactgttaaggattgataatactttttggagaatgttcagcaaggttatgtttgaatcaaaggttaagaaaaggataatgttatatgatgtaaaatttctatcattagtaggttatatagggtttaagaattggtttatagaacagttgagatcagctgagttgcatgaggtaccttggattgtcaatgccgaaggtgatctggttgagatcaagtcaattaaaatctatttgcaactgatagagcagagtttatttttaagaataactgttttgaactatacagatatggcacatgctctcacaagattaatcagaaagaagttgatgtgtgataatgcactattaacttcaattccatccccaatggttaacttaactcaagttattgatcctacagaacaattagcttatttccctaagataacatttgaaaggctaaaaaattatgacactagttcaaattatgctaaaggaaagctaacaaggaattacatgatactgttgccatggcaacatgttaatagatataactttgtctttagttctactggatgtaaagttagtctaaaaacatgcattggaaaacttatgaaagatctaaaccctaaagttctgtactttattggagaaggggcaggaaattggatggccagaacagcatgtgaatatcctgacatcaaatttgtatacagaagtttaaaagatgaccttgatcatcattatcctttggaataccagagagtgataggagaattaagcaggataatagatagtggtgaagggctttcaatggaaacaacagatgcaactcaaaaaactcattgggatttgatacacagagtaagcaaagatgctttattaataactttatgtgatgcagaatttaaggacagagatgatttttttaagatggtaattctatggaggaaacatgtattatcatgcagaatttgcactacctatgggacagacctctatttattcgcaaagtatcatgctaaagactgcaatataaaattacctttttttgtgagatcagttgccacctttattatgcaaggtagtaaactgtcaggctcggaatgctacatactcttaacactaggccaccacaacaatttaccttgtcatggagaaatacaaaattctaagatgaaaatagcagcgtgtaatgatttttatgctgcaaaaaaacttgacaataaatcaattgaagccaactgtaaatcacttttatcagggctaagaataccgataaataagaaggaattaaatagacagagaaggttattaacactacaaagcaaccattcttctgtagcaacagttggaggtagcaaggtcatagagtctaaatggttaacaaacaaggcaaacacaataattgattggttagaacatattttaaattctccaaaaggtgaattaaattatgatttttttgaagcattagaaaatacttaccctaatatgattaaactaatagataatctagggaatgcagagattaaaaaactgatcaaagtaactggatatatgcttgtaagtaaaaaatgaaaaatgataaaaatgataaaataggtgacaacttcatactattccaaagtaatcatttgattatgcaattatgtaatagttaattaaaaactaaaaatcaaaagttaaaaactaataactgtcattaagtttattaaaaataagaaattataattggatgtatacggtttttttgccgt

SEQ ID NO: 2 - DNA, 12645 bp, Human metapneumovirus
  source 1..12645: mol_type = genomic DNA
gcgaaaaaaacgcgtataaattagattacaaaaaaatatgggacaagtgaaaatgtctcttcaagggattcacctgagtgatctatcatacaagcatgctatattaaaagagtctcagtacacaataaaaagagatgtgggtacaacaactgcagtgacaccctcatcattgcaacaagaaataacgctgttgtgtggagaaattctgtatgctaaacatgctgattacaaatatgctgcagaaataggaatacaatatattagcacagctttaggatcagagagagtgcagcagattctgaggaactcaggcagtgaagtccaagtggtcttaaccagaacgtactctctggggaaagttaaaaacaataaaggagaagatttacagatgttagacatacacggggtagagaagagctgggtagaagagatagacaaagaagcaaggaaaacaatggcaaccttgcttaaggaatcatcaggtaatatcccacaaaatcagaggccctcagcaccagacacacccataatcttattatgtgtaggtgcattaatatttactaagctagcatcaaccatagaagtgggactagagaccacagtcagaagggctaaccgtgtactaagtgatgcactcaagagataccctagaatggacatcccaaaaattgccagatccttctatgacttatttgaacaaaaagtgtatcacagaagtttgttcattgagtatggcaaagcattaggctcatcatctacaggcagcaaagcagaaagtctatttgttaatatattcatgcaagcttatggagccggtcaaacaatgctaaggtggggggtcattgccaggtcatccaacaatataatgttaggacatgtatctgtccaagctgagttaaaacaggtcacagaagtctatgacttggtgcgagaaatgggccctgaatctggacttctacatttaaggcaaagcccaaaagctggactgttatcactagccaactgtcccaactttgcaagtgttgttctcggaaatgcctcaggcttaggcataatcggtatgtatcgtgggagagtaccaaacacagaattattttcagcagcagaaagttatgccaaaagtttgaaagagagcaataaaatcaatttctcttcattaggacttacagatgaagagaaagaggctgcagaacatttcttaaatgtgagtgacgacagtcaaaatgattatgagtaattaaaaaagtgggacaagtcaaaatgtctttccctgaaggaaaagatattcttttcatgggtaatgaagcagcaaaattagcagaagctttccagaaatcattaaggaaaccaagtcataaaagatctcaatctattataggagaaaaagtgaacactgtatcagaaacattggaattacctactatcagtagacctgcaaaaccaaccatactgtcagaaccaaagttagcatggactgataaaggtggggcaatcaaaactgaaataaagcaagcaatcaaagtcatggatcctattgaggaagaagagtctactgagaagaaggtgctgccctccagtgatgggaaaacccctgcagaaaagaaactgaaaccatcaactaacaccaaaaagaaagtttcgtttacaccaaatgaaccaggaaaatatacaaagttggaaaaagatgctctagatttgctctcagataatgaagaagaagatgcagaatcttcaatcttaacctttgaagaaagagatacttcatcgttaagcattgaggccagattggaatcaatagaggagaaattaagcatgatattagggctattaagaacactcaacattgctacagcaggacccacagcagcaagagatgggatcagagatgcaatgattggcgtaagagaggaattaatagcagacataataaaggaagccaaagggaaagcagcagaaatgatggaagaggaaatgagtcaacgatcaaaaataggaaacggtagtgtaaaactaacagagaaagcaaaagagcttaacaaaattgttgaagatgaaagcacaagtggagaatctgaagaagaagaagaaccaaaagacatacaagacaatagtcaagaagatgacatttaccagttaattatgtagtttaataaaaataaacaatgggacaagtaaaaatggagtcctacctagtagacacttatcaaggcattccttacacagcagctgttcaagttgatctaatagaaaaggacctgttacctgcaagcctaacaatatggttccctttgtttcaggccaacacaccaccagcagtgctgctcgatcagttgaaaaccctaacaataaccactctgtatgctgcatcacaaaatggtccaatactcaaagtgaatgcatcagcccaaggtgcagcaatgtctgtacttcccaaaaaatttgaagtcaatgcgactgtagcactcgatgaatatagcaaattggaatttgacaaactcacagtctgtgaagtaaaaacagtttacttaacaaccatgaaaccatacgggatggtatcaaaatttgtgagctcagccaaatcagttggcaaaaaaacacatgatctaatcgcactgtgtgattttatggatctagaaaagaacacacctgttacaataccagcattcatcaaatcagtttcaatcaaagagagtgagtcagctactgttgaagctgctataagcagtgaagcagaccaagctctaacacaggccaaaattgcaccttatgcgggattgattatgatcatgactatgaacaatcccaaaggcatattcaaaaagcttggagctgggactcaagttatagtagaactaggagcatatgtccaggctgaaagcataagtaaaatatgcaagacttggagccatcaagggacaagatatgtgttgaagtccagataacagccaagcaccttggccaagagctactaactctatctcatagattataaagtcaccattctagttatataaaaatcaagttagaacaagaattaaatcaatcaagaatgggacaaataaaaatgtcttggaaagtggtgatcattttttcattgttaataacacctcaacacggtcttaaagagagctatttagaagagtcatgtagcactataactgaaggatatctcagtgttctgaggacaggttggtataccaacgtttttacactggaggtaggtgatgtagagaaccttacatgtgctgatggacctagcttaataaaaacagaattagacctgaccaaaagtgcactaagagaactcagaacagtttctgctgatcaactggcaagagaggagcaaattgagaatcccagacaatctagatttgttctaggagcaatagcactcggtgttgcaacagcagctgcagttacagcaggtgttgcaattgccaaaaccatccggcttgaaagtgaagtaacagcaattaagaatgccctcaaaaagaccaatgaagcagtatctacattggggaatggagttcgagtgttggcaactgcagtgagggagctggaagattttgtgagcaagaatctaacacgtgcaatcaacaaaaacaagtgcgacattgctgacctgaaaatggccgttagcttcagtcaattcaacagaaggtttctaaatgttgtgcggcaattttcagacaatgctggaataacaccagcaatatccttggacttaatgacagatgctgaactagccagagctgtttccaacatgccaacatctgcaggacaaataaaactgatgttggagaaccgtgcaatggtaagaagaaaggggttcggaatcctgataggagtttacggaagctccgtaatttacatggtgcaactgccaatctttggagttatagacacgccttgctggatagtaaaagcggccccttcttgctcagaaaaaaagggaaactatgcttgccttttaagagaagatcaaggatggtattgtcagaatgcagggtcaactgtttactacccaaatgaaaaagactgcgaaacaagaggagaccatgtcttttgcgacacagcagcaggaatcaatgttgctgagcagtcaaaggagtgcaacatcaacatatccactactaattacccatgcaaagttagcacaggaagacaccctatcagtatggttgcactgtctcctcttggggctttggttgcttgctacaagggagtgagctgttccattggcagcaacagagtagggatcatcaagcaactgaacaaaggctgctcttatataaccaaccaagacgcagacacagtgacaatagacaacactgtataccagctaagcaaagttgagggcgaacagcatgttataaaaggaaggccagtgtcaagcagctttgatccagtcaaatttcctgaagatcaattcaatgttgcacttgaccaagttttcgaaagcattgagaacagtcaggccttggtggatcaatcaaacagaatcctaagcagtgcagagaaaggaaacactggcttcatcattgtaataattctaattgctgtccttggctctaccatgatcctagtgagtgtttttatcataataaagaaaacaaagaaacccacaggagcacctccagagctgagtggtgtcacaaacaatggcttcataccacataattagttaattaaaaataaagtaaattaaattaaaataaaataaaattaaaattaaaataaaataaaaataaaaatttgggacaaatcataatgtctcgcaaggctccatgcaaatatgaagtgcggggcaaatgcaatagaggaagtgagtgcaagtttaaccacaattactggagttggccagatagatacttactaataagatcaaattatttattaaatcaacttttaaggaacactgatagagctgatggcttatcaataatatcaggagcaggcagagaagataggacacaagattttgtcctaggttccaccaatgtggttcaaggttatattgatgataaccaaagcataacaaaagctgcagcctgttacagtctacataatataatcaaacaactacaagaagttgaagttaggcaggctagagataacaaaccatctgacagcaaacatgtggcacttcacaacttagtcctatcttatatggagatgagcaaaattcctgcatctttaatcaacaatctcaaaagactgccgagagagaaactgaaaaaattagcaaagcttataattgacttatcagcaggtgctgaaaatgactcttcatatgccttgcaagacagtgaaagcactaatcaagtgcagtgagcatggtcctgttttcattactatagaggttgattacatgatatggactcataaggacttaaaagaagctttatctaatgggatagtgaagtctcatactaacatttacaattgttatttagaaaacatagaaattatatatgtcaaggcttacttaagttagtaaaaacacatcagagtgggacaagtagttatggaggtgaaagtggagaacattcgaacaatagatatgctcaaagcaagagtgaaaaatcgtgtggcacgcagcaaatgctttaaaaatgcctctttgatcctaataggaataactacattgagtatagccctcaatatctatctaatcataaactatacaatgcaagaaaacacatccgaatcagaacatcacaccagctcatcacccatggaatccagcagggaaactccaacagtccctatggacaactcagacaccaatccaggctcacagtatccaactcaacagtccacagaaggctccacactctactttgcagcctcagcaagctcaccagagacagaaccaacatcaacaccagacacaacaagccgcccgcccttcgtcgacacacacacaacactaccaagtgcaagcagaacaaggacaagtccggcagtccacacaaaaaacaatccaaggacaagccccagaacacattccccaccatgggcaatgacaaggacggtccgtggaaccaccactctccgcacaagcagcacaagaaaaagaccgtccacagcatcagtccaacctgacagcagcgcaacaacccacaaacacgaagaagcaagcccagtgagcccgcaaacatctgcgagcacagcaagaccacaaaggaagggcatggaggccagcacatcaacaacatacaaccaaactagttaacaaaaaatacaaaataactctaagataaaccatatagacaccaacaattgagaagccaaaaggcaattcacaatctctccaaaaaggcaacaacaccatattagctccgcttaaatctccctggaaaaaacactcgcccatataccaactataccacaaccatcccaagaaaaaaagctgggtaaaacaacacccaagagacaaataacaatggatcctcttaatgaatccactgttaatgtctatcttcccgactcatatcttaaaggagtgatttcttttagtgagactaatgcaattggttcatgtctcttaaaaagaccttacctaaaaaatgacaacactgcaaaagttgccatagagaatcctgttatcgagcatgttagactcaaaaatgcaatcaattctaagatgaaaatatcagattacaggatagtagagccagtaaacatgcaacatgaaattatgaagaatgtacacagttgtgagctcacattattaaaacagtttttaacaaggagtaaaaatattagcactcttaaattaaatatgatatgtgattggctgcagttaaagtctacatcagatgatacctcaatcttaagttttatagatgtagaatttatacctagctgggtaagcaattggtttagtaattggtacaatctcaacaagttgattctggaattcaggaaagaagaagtaataagaactggttcaatcttgtgtaggtcattgggtaaattagtttttgttgtatcatcatacggatgtatagtcaagagcaacaaaagcaaaagagtgagcttcttcacatacaatcaactgttaacatggaaagatgtgatgttaagtagattcaatgcaaatttctgtatatgggtaagcaacagtctgaatgaaaatcaagaagggttagggttgagaagtaatctgcaaggcatattaactaataagctatatgaaactgtagattatatgcttagtttgtgttgcaatgaaggtttctcacttgtgaaagagtttgagggttttattatgagtgaaatccttaggattactgaacatgctcaattcagtactagatttagaaatactttattaaatggattaactgatcaattgacaaaattaaaaaataaaaacagactcagagttcatggtaccgtgttagaaaataatgattatccaatgtatgaagttgtacttaaattattaggagatactttgagatgtattaaattattaatcaataaaaacttagagaatgctgctgaattatactatatatttagaatattcggtcacccaatggtagatgaaagagatgcaatggatgctgtcaaattaaacaatgaaatcacaaaaatcctcaggttggagagcttgacagaactaagaggggcattcatattaaggattatcaaaggatttgtagacaacaacaaaagatggccgaaaattaaaaacttaaaagtgcttagtaaaagatggactatgtacttcaaagcaaaaagttaccctagtcaacttgaattaagtgaacaagattttttagagcttgctgcaatacagtttgaacaagagttttctgttcctgaaaaaaccaaccttgagatggtattaaatgataaagctatatcacctcctaaaagattaatttggtctgtgtacccaaaaaattacttacctgagacaataaaaaatcgatatctagaagagactttcaatgcaagtgatagtctcaaaacaagaagagtactagagtactatttgaaagataataaattcgaccaaaaagaacttaaaagttatgtggttaaacaagaatatttaaatgataaggatcatattgtctcgctaactggaaaagaaagagaattaagtgtaggtagaatgtttgctatgcaaccaggaaaacagcgacaaatacaaatattggctgaaaaattgttagctgataatattgtaccctttttcccagaaactttaacaaagtatggtgatctagatcttcagagaataatggaaatcaaatcagaactttcttctattaaaaccagaagaaatgatagttataataattacattgcaagagcatccatagtaacagatttaagtaagttcaaccaagcctttaggtatgaaactacagcgatctgtgcggatgtagcagatgaactacatggaacacaaagcctattctgttggttacatcttatcgttcctatgactacaatgatatgtgcctatagacatgcaccaccagaaacaaaaggtgaatatgatatagataagatagaagagcaaagtggtttatatagatatcatatgggtggtattgaaggatggtgtcaaaaactctggacaatggaagctatatctttattagatgttgtatctgtaaagacacgatgtcaaatgacatctttattaaacggtgacaaccaatcaatagatgtaagtaaaccagttaagttatctgagggtttagatgaagtgaaagcagattatagcttggctgtaaaaatgctaaaagaaataagagatgcatacagaaatataggccataaacttaaagaaggggaaacatatatatcaagagatcttcagtttataagtaaggtgattcaatctgaaggagtaatgcatcctacccctataaaaaagatcttaagagtgggaccatggataaacacaatattagatgacattaaaaccagtgcagagtcaatagggagtctatgtcaggaattagaatttaggggggaaagcataatagttagtctgatattaaggaatttttggctgtataatttatacatgcatgaatcaaagcaacaccccctagcagggaagcagttattcaaacaactaaataaaacattaacatcagtgcagagattttttgaaattaaaaaggaaaatgaagtagtagatctatggatgaacataccaatgcagtttggaggaggagatccagtagtcttctatagatctttctatagaaggacccctgattttttaactgaagcaatcagtcatgtagatattctgttaaaaatatcagccaacataagaaatgaagcgaaaataagtttcttcaaagccttactgtcaatagaaaaaaatgaacgtgctacactgacaacactaatgagagaccctcaagctgtgggctcagagcgacaagcaaaagtaacaagtgatatcaatagaacagcagttaccagcatcttaagtctttctccaaatcaacttttcagcgatagtgctatacactacagtagaaatgaagaagaggtcggaatcattgctgacaacataacacctgtttatcctcatggactgagagttttgtatgaatcattaccttttcataaagctgaaaaagttgtaaatatgatatcaggaacaaaatccataaccaacttattacagagaacatctgctattaatggtgaagatattgacagagctgtatccatgatgctggagaacctaggattattatctagaatattgtcagtagttgttgatagtatagaaattccaaccaaatctaatggtaggctgatatgttgtcagatatctagaaccctaagggagacatcatggaataatatggaaatagttggagtaacatcccctagcatcactacatgcatggatgtcatatatgcaactagctctcatttgaaagggataatcattgaaaagttcagcactgacagaactacaagaggtcaaagaggtccaaagagcccttgggtaggatcgagcacgcaagagaaaaaattagttcctgtttataacagacaaattctttcaaaacaacaaagagaacagctagaagcaattggaaaaatgagatgggtatataaagggacaccaggtttaagacgattactcaataagatttgtcttggaagtttaggcattagttacaaatgtgtaaaacctttattacctaggtttatgagtgtaaatttcctacacaggttatctgtcagtagtagacctatggaattcccagcatcagttccagcttatagaacaacaaattaccattttgacactagtcctattaatcaagcactaagtgaaagatttgggaatgaagatattaatttggtcttccaaaatgcaatcagctgtggaattagcataatgagtgtagtagaacaattaactggtaggagtccaaaacagttagttttaataccccaattagaagaaatagacattatgccaccaccagtgtttcaagggaaattcaattataagctagtagataagataacttctgaccaacatatcttcagtccagacaaaatagatatgttaacactggggaaaatgctcatgccaactataaaaggtcagaaaacagatcagttcttgaacaagagagagaattatttccatgggaacaatcttattgagtctttgtcagcagcgttagcatgtcattggtgtgggatattaacagagcaatgtatagaaaataatattttcaagaaagactggggtgacgggttcatatcggatcatgcttttatggacttcaaaatattcctatgtgtctttaaaactaaacttttatgtagttggggatcccaagggaaaaacattaaagatgaagatatagtagatgaatcgatagataaactgttaaggattgataatactttttggagaatgttcagcaaggttatgtttgaatcaaaggttaagaaaaggataatgttatatgatgtaaaatttctatcattagtaggttatatagggtttaagaattggtttatagaacagttgagatcagctgagttgcatgaggtaccttggattgtcaatgccgaaggtgatctggttgagatcaagtcaattaaaatctatttgcaactgatagagcagagtttatttttaagaataactgttttgaactatacagatatggcacatgctctcacaagattaatcagaaagaagttgatgtgtgataatgcactattaacttcaattccatccccaatggttaacttaactcaagttattgatcctacagaacaattagcttatttccctaagataacatttgaaaggctaaaaaattatgacactagttcaaattatgctaaaggaaagctaacaaggaattacatgatactgttgccatggcaacatgttaatagatataactttgtctttagttctactggatgtaaagttagtctaaaaacatgcattggaaaacttatgaaagatctaaaccctaaagttctgtactttattggagaaggggcaggaaattggatggccagaacagcatgtgaatatcctgacatcaaatttgtatacagaagtttaaaagatgaccttgatcatcattatcctttggaataccagagagtgataggagaattaagcaggataatagatagtggtgaagggctttcaatggaaacaacagatgcaactcaaaaaactcattgggatttgatacacagagtaagcaaagatgctttattaataactttatgtgatgcagaatttaaggacagagatgatttttttaagatggtaattctatggaggaaacatgtattatcatgcagaatttgcactacctatgggacagacctctatttattcgcaaagtatcatgctaaagactgcaatataaaattacctttttttgtgagatcagttgccacctttattatgcaaggtagtaaactgtcaggctcggaatgctacatactcttaacactaggccaccacaacaatttaccttgtcatggagaaatacaaaattctaagatgaaaatagcagcgtgtaatgatttttatgctgcaaaaaaacttgacaataaatcaattgaagccaactgtaaatcacttttatcagggctaagaataccgataaataagaaggaattaaatagacagagaaggttattaacactacaaagcaaccattcttctgtagcaacagttggaggtagcaaggtcatagagtctaaatggttaacaaacaaggcaaacacaataattgattggttagaacatattttaaattctccaaaaggtgaattaaattatgatttttttgaagcattagaaaatacttaccctaatatgattaaactaatagataatctagggaatgcagagattaaaaaactgatcaaagtaactggatatatgcttgtaagtaaaaaatgaaaaatgataaaaatgataaaataggtgacaacttcatactattccaaagtaatcatttgattatgcaattatgtaatagttaattaaaaactaaaaatcaaaagttaaaaactaataactgtcattaagtttattaaaaataagaaattataattggatgtatacggtttttttgccgt

SEQ ID NO: 3 - protein, 513 aa, Human orthopneumovirus
MELLILKANAITTILTAVTFCFASGQNITEEFYQSTCSAVSKGYLSALRTGWYTSVITIELSNIKKNKCNGTDAKVKLIKQELDKYKNAVTELQLLMQSTQATNNRARRELPRFMNYTLNNAKKTNVTLSKKRKRRFLGFLLGVGSAIASGVAVSKVLHLEGEVNKIKSALLSTNKAVVSLSNGVSVLTSKVLDLKNYIDKQVLPIVNKQSCSISNIETVIEFQQKNNRLLEITREFSVNAGVTTPVSTYMLTNSELLSLINDMPITNDQKKLMSNNVQIVRQQSYSIMSIIKEEVLAYVVQLPLYGVIDTPCWKLHTSPLCTTNTKEGSNICLTRTDRGWYCDNAGSVSFFPQAETCKVQSNRVFCDTMNSLTLPSEVNLCNVDIFNPKYDCKIMTSKTDVSSSVITSLGAIVSCYGKTKCTASNKNRGIIKTFSNGCDYVSNKGVDTVSVGNTLYYVNKQEGKSLYVKGEPIINFYDPLVFPSDEFDASISQVNEKINQSLAFIRKSDELL

SEQ ID NO: 4 - protein, 574 aa, Human orthopneumovirus
MELLILKANAITTILTAVTFCFASGQNITEEFYQSTCSAVSKGYLSALRTGWYTSVITIELSNIKKNKCNGTDAKVKLIKQELDKYKNAVTELQLLMQSTQATNNRARRELPRFMNYTLNNAKKTNVTLSKKRKRRFLGFLLGVGSAIASGVAVSKVLHLEGEVNKIKSALLSTNKAVVSLSNGVSVLTSKVLDLKNYIDKQVLPIVNKQSCSISNIETVIEFQQKNNRLLEITREFSVNAGVTTPVSTYMLTNSELLSLINDMPITNDQKKLMSNNVQIVRQQSYSIMSIIKEEVLAYVVQLPLYGVIDTPCWKLHTSPLCTTNTKEGSNICLTRTDRGWYCDNAGSVSFFPQAETCKVQSNRVFCDTMNSLTLPSEVNLCNVDIFNPKYDCKIMTSKTDVSSSVITSLGAIVSCYGKTKCTASNKNRGIIKTFSNGCDYVSNKGVDTVSVGNTLYYVNKQEGKSLYVKGEPIINFYDPLVFPSDEFDASISQVNEKINQSLAFIRKSDELLHNVNAGKSTTNIMITTIIIVIIVILLSLIAVGLLLYCKARSTPVTLSKDQLSGINNIAFSN

SEQ ID NO: 5 - protein, 513 aa, Human orthopneumovirus
MELLILKANAITTILTAVTFCFASGQNITEEFYQSTCSAVSKGYLGALRTGWYTSVITIELSNIKENKCNGTDAKVKLIKQELDKYKNAVTDLQLLMQSTPATNNRARRELPRFMNYTLNNAKKTNVTLSKKRKRRFLGFLLGVGSAICSGVAVCKVLHLEGEVNKIKSALLSTNKAVVSLSNGVSVLTFKVLDLKNYIDKQLLPILNKQSCSIPNIETVIEFQQKNNRLLEITREFSVNAGVTTPVSTYMLTNSELLSLINDMPITNDQKKLMSNNVQIVRQQSYSIMCIIKEEVLAYVVQLPLYGVIDTPCWKLHTSPLCTTNTKEGSNICLTRTDRGWYCDNAGSVSFFPQAETCKVQSNRVFCDTMNSRTLPSEVNLCNVDIFNPKYDCKIMTSKTDVSSSVITSLGAIVSCYGKTKCTASNKNRGIIKTFSNGCDYVSNKGVDTVSVGNTLYCVNKQEGQSLYVKGEPIINFYDPLVFPSDEFDASISQVNEKINQSLAFIRKSDELL

SEQ ID NO: 6 - protein, 574 aa, Human orthopneumovirus
MELLILKANAITTILTAVTFCFASGQNITEEFYQSTCSAVSKGYLGALRTGWYTSVITIELSNIKENKCNGTDAKVKLIKQELDKYKNAVTDLQLLMQSTPATNNRARRELPRFMNYTLNNAKKTNVTLSKKRKRRFLGFLLGVGSAICSGVAVCKVLHLEGEVNKIKSALLSTNKAVVSLSNGVSVLTFKVLDLKNYIDKQLLPILNKQSCSIPNIETVIEFQQKNNRLLEITREFSVNAGVTTPVSTYMLTNSELLSLINDMPITNDQKKLMSNNVQIVRQQSYSIMCIIKEEVLAYVVQLPLYGVIDTPCWKLHTSPLCTTNTKEGSNICLTRTDRGWYCDNAGSVSFFPQAETCKVQSNRVFCDTMNSRTLPSEVNLCNVDIFNPKYDCKIMTSKTDVSSSVITSLGAIVSCYGKTKCTASNKNRGIIKTFSNGCDYVSNKGVDTVSVGNTLYCVNKQEGQSLYVKGEPIINFYDPLVFPSDEFDASISQVNEKINQSLAFIRKSDELLHNVNAGKSTTNIMITTIIIVIIVILLSLIAVGLLLYCKARSTPVTLSKDQLSGINNIAFSN

SEQ ID NO: 7 - protein, 539 aa, Human metapneumovirus
MSWKVVIIFSLLITPQHGLKESYLEESCSTITEGYLSVLRTGWYTNVFTLEVGDVENLTCADGPSLIKTELDLTKSALRELRTVSADQLAREEQIENPRQSRFVLGAIALGVATAAAVTAGVAIAKTIRLESEVTAIKNALKKTNEAVSTLGNGVRVLATAVRELEDFVSKNLTRAINKNKCDIADLKMAVSFSQFNRRFLNVVRQFSDNAGITPAISLDLMTDAELARAVSNMPTSAGQIKLMLENRAMVRRKGFGILIGVYGSSVIYMVQLPIFGVIDTPCWIVKAAPSCSEKKGNYACLLREDQGWYCQNAGSTVYYPNEKDCETRGDHVFCDTAAGINVAEQSKECNINISTTNYPCKVSTGRHPISMVALSPLGALVACYKGVSCSIGSNRVGIIKQLNKGCSYITNQDADTVTIDNTVYQLSKVEGEQHVIKGRPVSSSFDPVKFPEDQFNVALDQVFESIENSQALVDQSNRILSSAEKGNTGFIIVIILIAVLGSTMILVSVFIIIKKTKKPTGAPPELSGVTNNGFIPHN

SEQ ID NO: 8 - protein, 571 aa, synthetic construct
  REGION 1..571: note = Chimeric protein hRSV/hMPV
MELLILKANAITTILTAVTFCFASGQNITEEFYQSTCSAVSKGYLGALRTGWYTSVITIELSNIKENKCNGTDAKVKLIKQELDKYKNAVTDLQLLMQSTPATNNRARRELPRFMNYTLNNAKKTNVTLSKKRKRRFLGFLLGVGSAICSGVAVCKVLHLEGEVNKIKSALLSTNKAVVSLSNGVSVLTFKVLDLKNYIDKQLLPILNKQSCSIPNIETVIEFQQKNNRLLEITREFSVNAGVTTPVSTYMLTNSELLSLINDMPITNDQKKLMSNNVQIVRQQSYSIMCIIKEEVLAYVVQLPLYGVIDTPCWKLHTSPLCTTNTKEGSNICLTRTDRGWYCDNAGSVSFFPQAETCKVQSNRVFCDTMNSRTLPSEVNLCNVDIFNPKYDCKIMTSKTDVSSSVITSLGAIVSCYGKTKCTASNKNRGIIKTFSNGCDYVSNKGVDTVSVGNTLYCVNKQEGQSLYVKGEPIINFYDPLVFPSDEFDASISQVNEKINQSLAFIRKSDELLSSAEKGNTGFIIVIILIAVLGSTMILVSVFIIIKKTKKPTGAPPELSGVTNNGFIPHN

SEQ ID NO: 9 - protein, 571 aa, synthetic construct
  REGION 1..571: note = Chimeric protein hRSV/hMPV
MELLILKANAITTILTAVTFCFASGQNITEEFYQSTCSAVSKGYLSALRTGWYTSVITIELSNIKKNKCNGTDAKVKLIKQELDKYKNAVTELQLLMQSTQATNNRARRELPRFMNYTLNNAKKTNVTLSKKRKRRFLGFLLGVGSAIASGVAVSKVLHLEGEVNKIKSALLSTNKAVVSLSNGVSVLTSKVLDLKNYIDKQVLPIVNKQSCSISNIETVIEFQQKNNRLLEITREFSVNAGVTTPVSTYMLTNSELLSLINDMPITNDQKKLMSNNVQIVRQQSYSIMSIIKEEVLAYVVQLPLYGVIDTPCWKLHTSPLCTTNTKEGSNICLTRTDRGWYCDNAGSVSFFPQAETCKVQSNRVFCDTMNSLTLPSEVNLCNVDIFNPKYDCKIMTSKTDVSSSVITSLGAIVSCYGKTKCTASNKNRGIIKTFSNGCDYVSNKGVDTVSVGNTLYYVNKQEGKSLYVKGEPIINFYDPLVFPSDEFDASISQVNEKINQSLAFIRKSDELLSSAEKGNTGFIIVIILIAVLGSTMILVSVFIIIKKTKKPTGAPPELSGVTNNGFIPHN

SEQ ID NO: 10 - DNA, 15160 bp, synthetic construct
  misc_feature 1..15160: note = hMPV + exogenous DNA sequence + GFP
  source 1..15160: mol_type = other DNA
acgcgaaaaaaacgcgtataaattagattacaaaaaaatatgggacaagtgaaaatggtgagcaagggcgaggagctgttcaccggggtggtgcccatcctggtcgagctggacggcgacgtaaacggccacaagttcagcgtgtccggcgagggcgagggcgatgccacctacggcaagctgaccctgaagttcatctgcaccaccggcaagctgcccgtgccctggcccaccctcgtgaccaccctgacctacggcgtgcagtgcttcagccgctaccccgaccacatgaagcagcacgacttcttcaagtccgccatgcccgaaggctacgtccaggagcgcaccatcttcttcaaggacgacggcaactacaagacccgcgccgaggtgaagttcgagggcgacaccctggtgaaccgcatcgagctgaagggcatcgacttcaaggaggacggcaacatcctggggcacaagctggagtacaactacaacagccacaacgtctatatcatggccgacaagcagaagaacggcatcaaggtgaacttcaagatccgccacaacatcgaggacggcagcgtgcagctcgccgaccactaccagcagaacacccccatcggcgacggccccgtgctgctgcccgacaaccactacctgagcacccagtccgccctgagcaaagaccccaacgagaagcgcgatcacatggtcctgctggagttcgtgaccgccgccgggatcactctcggcatggacgagctgtacaagtaagttaattaaaaagtgggacaagtgaaaatgtctcttcaagggattcacctgagtgatctatcatacaagcatgctatattaaaagagtctcagtacacaataaaaagagatgtgggtacaacaactgcagtgacaccctcatcattgcaacaagaaataacgctgttgtgtggagaaattctgtatgctaaacatgctgattacaaatatgctgcagaaataggaatacaatatattagcacagctttaggatcagagagagtgcagcagattctgaggaactcaggcagtgaagtccaagtggtcttaaccagaacgtactctctggggaaagttaaaaacaataaaggagaagatttacagatgttagacatacacggggtagagaagagctgggtagaagagatagacaaagaagcaaggaaaacaatggcaaccttgcttaaggaatcatcaggtaatatcccacaaaatcagaggccctcagcaccagacacacccataatcttattatgtgtaggtgcattaatatttactaagctagcatcaaccatagaagtgggactagagaccacagtcagaagggctaaccgtgtactaagtgatgcactcaagagataccctagaatggacatcccaaaaattgccagatccttctatgacttatttgaacaaaaagtgtatcacagaagtttgttcattgagtatggcaaagcattaggctcatcatctacaggcagcaaagcagaaagtctatttgttaatatattcatgcaagcttatggagccggtcaaacaatgctaaggtggggggtcattgccaggtcatccaacaatataatgttaggacatgtatctgtccaagctgagttaaaacaggtcacagaagtctatgacttggtgcgagaaatgggccctgaatctggacttctacatttaaggcaaagcccaaaagctggactgttatcactagccaactgtcccaactttgcaagtgttgttctcggaaatgcctcaggcttaggcataatcggtatgtatcgtgggagagtaccaaacacagaattattttcagcagcagaaagttatgccaaaagtttgaaagagagcaataaaatcaatttctcttcattaggacttacagatgaagagaaagaggctgcagaacatttcttaaatgtgagtgacgacagtcaaaatgattatgagtaattaaaaaagtgggacaagtcaaaatgtctttccctgaaggaaaagatattcttttcatgggtaatgaagcagcaaaattagcagaagctttccagaaatcattaaggaaaccaagtcataaaagatctcaatctattataggagaaaaagtgaacactgtatcagaaacattggaattacctactatcagtagacctgcaaaaccaaccatactgtcagaaccaaagttagcatggactgataaaggtggggcaatcaaaactgaaataaagcaagcaatcaaagtcatggatcctattgaggaagaagagtctactgagaagaaggtgctgccctccagtgatgggaaaacccctgcagaaaagaaactgaaaccatcaactaacaccaaaaagaaagtttcgtttacaccaaatgaaccaggaaaatatacaaagttggaaaaagatgctctagatttgctctcagataatgaagaagaagatgcagaatcttcaatcttaacctttgaagaaagagatacttcatcgttaagcattgaggccagattggaatcaatagaggagaaattaagcatgatattagggctattaagaacactcaacattgctacagcaggacccacagcagcaagagatgggatcagagatgcaatgattggcgtaagagaggaattaatagcagacataataaaggaagccaaagggaaagcagcagaaatgatggaagaggaaatgagtcaacgatcaaaaataggaaacggtagtgtaaaactaacagagaaagcaaaagagcttaacaaaattgttgaagatgaaagcacaagtggagaatctgaagaagaagaagaaccaaaagacatacaagacaatagtcaagaagatgacatttaccagttaattatgtagtttaataaaaataaacaatgggacaagtaaaaatggagtcctacctagtagacacttatcaaggcattccttacacagcagctgttcaagttgatctaatagaaaaggacctgttacctgcaagcctaacaatatggttccctttgtttcaggccaacacaccaccagcagtgctgctcgatcagttgaaaaccctaacaataaccactctgtatgctgcatcacaaaatggtccaatactcaaagtgaatgcatcagcccaaggtgcagcaatgtctgtacttcccaaaaaatttgaagtcaatgcgactgtagcactcgatgaatatagcaaattggaatttgacaaactcacagtctgtgaagtaaaaacagtttacttaacaaccatgaaaccatacgggatggtatcaaaatttgtgagctcagccaaatcagttggcaaaaaaacacatgatctaatcgcactgtgtgattttatggatctagaaaagaacacacctgttacaataccagcattcatcaaatcagtttcaatcaaagagagtgagtcagctactgttgaagctgctataagcagtgaagcagaccaagctctaacacaggccaaaattgcaccttatgcgggattgattatgatcatgactatgaacaatcccaaaggcatattcaaaaagcttggagctgggactcaagttatagtagaactaggagcatatgtccaggctgaaagcataagtaaaatatgcaagacttggagccatcaagggacaagatatgtgttgaagtccagataacagccaagcaccttggccaagagctactaactctatctcatagattataaagtcaccattctagttatataaaaatcaagttagaacaagaattaaatcaatcaagaatgggacaaataaaaatgtcttggaaagtggtgatcattttttcattgttaataacacctcaacacggtcttaaagagagctatttagaagagtcatgtagcactataactgaaggatatctcagtgttctgaggacaggttggtataccaacgtttttacactggaggtaggtgatgtagagaaccttacatgtgctgatggacctagcttaataaaaacagaattagacctgaccaaaagtgcactaagagaactcagaacagtttctgctgatcaactggcaagagaggagcaaattgagaatcccagacaatctagatttgttctaggagcaatagcactcggtgttgcaacagcagctgcagttacagcaggtgttgcaattgccaaaaccatccggcttgaaagtgaagtaacagcaattaagaatgccctcaaaaagaccaatgaagcagtatctacattggggaatggagttcgagtgttggcaactgcagtgagggagctggaagattttgtgagcaagaatctaacacgtgcaatcaacaaaaacaagtgcgacattgctgacctgaaaatggccgttagcttcagtcaattcaacagaaggtttctaaatgttgtgcggcaattttcagacaatgctggaataacaccagcaatatccttggacttaatgacagatgctgaactagccagagctgtttccaacatgccaacatctgcaggacaaataaaactgatgttggagaaccgtgcaatggtaagaagaaaggggttcggaatcctgataggagtttacggaagctccgtaatttacatggtgcaactgccaatctttggagttatagacacgccttgctggatagtaaaagcggccccttcttgctcagaaaaaaagggaaactatgcttgccttttaagagaagatcaaggatggtattgtcagaatgcagggtcaactgtttactacccaaatgaaaaagactgcgaaacaagaggagaccatgtcttttgcgacacagcagcaggaatcaatgttgctgagcagtcaaaggagtgcaacatcaacatatccactactaattacccatgcaaagttagcacaggaagacaccctatcagtatggttgcactgtctcctcttggggctttggttgcttgctacaagggagtgagctgttccattggcagcaacagagtagggatcatcaagcaactgaacaaaggctgctcttatataaccaaccaagacgcagacacagtgacaatagacaacactgtataccagctaagcaaagttgagggcgaacagcatgttataaaaggaaggccagtgtcaagcagctttgatccagtcaaatttcctgaagatcaattcaatgttgcacttgaccaagttttcgaaagcattgagaacagtcaggccttggtggatcaatcaaacagaatcctaagcagtgcagagaaaggaaacactggcttcatcattgtaataattctaattgctgtccttggctctaccatgatcctagtgagtgtttttatcataataaagaaaacaaagaaacccacaggagcacctccagagctgagtggtgtcacaaacaatggcttcataccacataattagttaattaaaaataaaaacacatcagagtgggacaagtgaaaatggagttgctaatcctcaaagcaaatgcaattaccacaatcctcactgcagtcacattttgttttgcttctggtcaaaacatcactgaagaattttatcaatcaacatgcagtgcagttagcaaaggctatcttagtgctctgagaactggttggtataccagtgttataactatagaattaagtaatatcaagaaaaataagtgtaatggaacagatgctaaggtaaaattgataaaacaagaattagataaatataaaaatgctgtaacagaattgcagttgctcatgcaaagcacacaagcaacaaacaatcgagccagaagagaactaccaaggtttatgaattatacactcaacaatgccaaaaaaaccaatgtaacattaagcaagaaaaggaaaagaagatttcttggttttttgttaggtgttggatctgcaatcgccagtggcgttgctgtatctaaggtcctgcacctagaaggggaagtgaacaagatcaaaagtgctctactatccacaaacaaggctgtagtcagcttatcaaatggagttagtgtcttaaccagcaaagtgttagacctcaaaaactatatagataaacaagtgttacctattgtgaacaagcaaagctgcagcatatcaaatatagaaactgtgatagagttccaacaaaagaacaacagactactagagattaccagggaatttagtgttaatgcaggtgtaactacacctgtaagcacttacatgttaactaatagtgaattattgtcattaatcaatgatatgcctataacaaatgatcagaaaaagttaatgtccaacaatgttcaaatagttagacagcaaagttactctatcatgtccataataaaagaggaagtcttagcatatgtagtacaattaccactatatggtgttatagatacaccctgttggaaactacacacatcccctctatgtacaaccaacacaaaagaagggtccaacatctgtttaacaagaactgacagaggatggtactgtgacaatgcaggatcagtatctttcttcccacaagctgaaacatgtaaagttcaatcaaatcgagtattttgtgacacaatgaacagtttaacattaccaagtgaagtaaatctctgcaatgttgacatattcaaccccaaatatgattgtaaaattatgacttcaaaaacagatgtaagcagctccgttatcacatctctaggagccattgtgtcatgctatggcaaaactaaatgtacagcatccaataaaaatcgtggaatcataaagacattttctaacgggtgcgattatgtatcaaataaaggggtggacactgtgtctgtaggtaacacattatattatgtaaataagcaagaaggtaaaagtctctatgtaaaaggtgaaccaataataaatttctatgacccattagtattcccctctgatgaatttgatgcatcaatatctcaagtcaacgagaagattaaccagagcctagcatttattcgtaaatccgatgaattattacataatgtaaatgctggtaaatccaccacaaatatcatgataactactataattatagtgattatagtaatattgttatcattaattgctgttggactgctcttatactgtaaggccagaagcacaccagtcacactaagcaaagatcaactgagtggtataaataatattgcatttagtaactaattaattaaaaataaagtaaattaaattaaaataaaataaaattaaaattaaaataaaataaaaataaaaatttgggacaaatcataatgtctcgcaaggctccatgcaaatatgaagtgcggggcaaatgcaatagaggaagtgagtgcaagtttaaccacaattactggagttggccagatagatacttactaataagatcaaattatttattaaatcaacttttaaggaacactgatagagctgatggcttatcaataatatcaggagcaggcagagaagataggacacaagattttgtcctaggttccaccaatgtggttcaaggttatattgatgataaccaaagcataacaaaagctgcagcctgttacagtctacataatataatcaaacaactacaagaagttgaagttaggcaggctagagataacaaaccatctgacagcaaacatgtggcacttcacaacttagtcctatcttatatggagatgagcaaaattcctgcatctttaatcaacaatctcaaaagactgccgagagagaaactgaaaaaattagcaaagcttataattgacttatcagcaggtgctgaaaatgactcttcatatgccttgcaagacagtgaaagcactaatcaagtgcagtgagcatggtcctgttttcattactatagaggttgattacatgatatggactcataaggacttaaaagaagctttatctaatgggatagtgaagtctcatactaacatttacaattgttatttagaaaacatagaaattatatatgtcaaggcttacttaagttagtaaaaacacatcagagtgggacaagtagttatggaggtgaaagtggagaacattcgaacaatagatatgctcaaagcaagagtgaaaaatcgtgtggcacgcagcaaatgctttaaaaatgcctctttgatcctaataggaataactacattgagtatagccctcaatatctatctaatcataaactatacaatgcaagaaaacacatccgaatcagaacatcacaccagctcatcacccatggaatccagcagggaaactccaacagtccctatggacaactcagacaccaatccaggctcacagtatccaactcaacagtccacagaaggctccacactctactttgcagcctcagcaagctcaccagagacagaaccaacatcaacaccagacacaacaagccgcccgcccttcgtcgacacacacacaacactaccaagtgcaagcagaacaaggacaagtccggcagtccacacaaaaaacaatccaaggacaagccccagaacacattccccaccatgggcaatgacaaggacggtccgtggaaccaccactctccgcacaagcagcacaagaaaaagaccgtccacagcatcagtccaacctgacagcagcgcaacaacccacaaacacgaagaagcaagcccagtgagcccgcaaacatctgcgagcacagcaagaccacaaaggaagggcatggaggccagcacatcaacaacatacaaccaaactagttaacaaaaaatacaaaataactctaagataaaccatatagacaccaacaattgagaagccaaaaggcaattcacaatctctccaaaaaggcaacaacaccatattagctccgcttaaatctccctggaaaaaacactcgcccatataccaactataccacaaccatcccaagaaaaaaagctgggtaaaacaacacccaagagacaaataacaatggatcctcttaatgaatccactgttaatgtctatcttcccgactcatatcttaaaggagtgatttcttttagtgagactaatgcaattggttcatgtctcttaaaaagaccttacctaaaaaatgacaacactgcaaaagttgccatagagaatcctgttatcgagcatgttagactcaaaaatgcaatcaattctaagatgaaaatatcagattacaggatagtagagccagtaaacatgcaacatgaaattatgaagaatgtacacagttgtgagctcacattattaaaacagtttttaacaaggagtaaaaatattagcactcttaaattaaatatgatatgtgattggctgcagttaaagtctacatcagatgatacctcaatcttaagttttatagatgtagaatttatacctagctgggtaagcaattggtttagtaattggtacaatctcaacaagttgattctggaattcaggaaagaagaagtaataagaactggttcaatcttgtgtaggtcattgggtaaattagtttttgttgtatcatcatacggatgtatagtcaagagcaacaaaagcaaaagagtgagcttcttcacatacaatcaactgttaacatggaaagatgtgatgttaagtagattcaatgcaaatttctgtatatgggtaagcaacagtctgaatgaaaatcaagaagggttagggttgagaagtaatctgcaaggcatattaactaataagctatatgaaactgtagattatatgcttagtttgtgttgcaatgaaggtttctcacttgtgaaagagtttgagggttttattatgagtgaaatccttaggattactgaacatgctcaattcagtactagatttagaaatactttattaaatggattaactgatcaattgacaaaattaaaaaataaaaacagactcagagttcatggtaccgtgttagaaaataatgattatccaatgtatgaagttgtacttaaattattaggagatactttgagatgtattaaattattaatcaataaaaacttagagaatgctgctgaattatactatatatttagaatattcggtcacccaatggtagatgaaagagatgcaatggatgctgtcaaattaaacaatgaaatcacaaaaatcctcaggttggagagcttgacagaactaagaggggcattcatattaaggattatcaaaggatttgtagacaacaacaaaagatggccgaaaattaaaaacttaaaagtgcttagtaaaagatggactatgtacttcaaagcaaaaagttaccctagtcaacttgaattaagtgaacaagattttttagagcttgctgcaatacagtttgaacaagagttttctgttcctgaaaaaaccaaccttgagatggtattaaatgataaagctatatcacctcctaaaagattaatttggtctgtgtacccaaaaaattacttacctgagacaataaaaaatcgatatctagaagagactttcaatgcaagtgatagtctcaaaacaagaagagtactagagtactatttgaaagataataaattcgaccaaaaagaacttaaaagttatgtggttaaacaagaatatttaaatgataaggatcatattgtctcgctaactggaaaagaaagagaattaagtgtaggtagaatgtttgctatgcaaccaggaaaacagcgacaaatacaaatattggctgaaaaattgttagctgataatattgtaccctttttcccagaaactttaacaaagtatggtgatctagatcttcagagaataatggaaatcaaatcagaactttcttctattaaaaccagaagaaatgatagttataataattacattgcaagagcatccatagtaacagatttaagtaagttcaaccaagcctttaggtatgaaactacagcgatctgtgcggatgtagcagatgaactacatggaacacaaagcctattctgttggttacatcttatcgttcctatgactacaatgatatgtgcctatagacatgcaccaccagaaacaaaaggtgaatatgatatagataagatagaagagcaaagtggtttatatagatatcatatgggtggtattgaaggatggtgtcaaaaactctggacaatggaagctatatctttattagatgttgtatctgtaaagacacgatgtcaaatgacatctttattaaacggtgacaaccaatcaatagatgtaagtaaaccagttaagttatctgagggtttagatgaagtgaaagcagattatagcttggctgtaaaaatgctaaaagaaataagagatgcatacagaaatataggccataaacttaaagaaggggaaacatatatatcaagagatcttcagtttataagtaaggtgattcaatctgaaggagtaatgcatcctacccctataaaaaagatcttaagagtgggaccatggataaacacaatattagatgacattaaaaccagtgcagagtcaatagggagtctatgtcaggaattagaatttaggggggaaagcataatagttagtctgatattaaggaatttttggctgtataatttatacatgcatgaatcaaagcaacaccccctagcagggaagcagttattcaaacaactaaataaaacattaacatcagtgcagagattttttgaaattaaaaaggaaaatgaagtagtagatctatggatgaacataccaatgcagtttggaggaggagatccagtagtcttctatagatctttctatagaaggacccctgattttttaactgaagcaatcagtcatgtagatattctgttaaaaatatcagccaacataagaaatgaagcgaaaataagtttcttcaaagccttactgtcaatagaaaaaaatgaacgtgctacactgacaacactaatgagagaccctcaagctgtgggctcagagcgacaagcaaaagtaacaagtgatatcaatagaacagcagttaccagcatcttaagtctttctccaaatcaacttttcagcgatagtgctatacactacagtagaaatgaagaagaggtcggaatcattgctgacaacataacacctgtttatcctcatggactgagagttttgtatgaatcattaccttttcataaagctgaaaaagttgtaaatatgatatcaggaacaaaatccataaccaacttattacagagaacatctgctattaatggtgaagatattgacagagctgtatccatgatgctggagaacctaggattattatctagaatattgtcagtagttgttgatagtatagaaattccaaccaaatctaatggtaggctgatatgttgtcagatatctagaaccctaagggagacatcatggaataatatggaaatagttggagtaacatcccctagcatcactacatgcatggatgtcatatatgcaactagctctcatttgaaagggataatcattgaaaagttcagcactgacagaactacaagaggtcaaagaggtccaaagagcccttgggtaggatcgagcacgcaagagaaaaaattagttcctgtttataacagacaaattctttcaaaacaacaaagagaacagctagaagcaattggaaaaatgagatgggtatataaagggacaccaggtttaagacgattactcaataagatttgtcttggaagtttaggcattagttacaaatgtgtaaaacctttattacctaggtttatgagtgtaaatttcctacacaggttatctgtcagtagtagacctatggaattcccagcatcagttccagcttatagaacaacaaattaccattttgacactagtcctattaatcaagcactaagtgaaagatttgggaatgaagatattaatttggtcttccaaaatgcaatcagctgtggaattagcataatgagtgtagtagaacaattaactggtaggagtccaaaacagttagttttaataccccaattagaagaaatagacattatgccaccaccagtgtttcaagggaaattcaattataagctagtagataagataacttctgaccaacatatcttcagtccagacaaaatagatatgttaacactggggaaaatgctcatgccaactataaaaggtcagaaaacagatcagttcttgaacaagagagagaattatttccatgggaacaatcttattgagtctttgtcagcagcgttagcatgtcattggtgtgggatattaacagagcaatgtatagaaaataatattttcaagaaagactggggtgacgggttcatatcggatcatgcttttatggacttcaaaatattcctatgtgtctttaaaactaaacttttatgtagttggggatcccaagggaaaaacattaaagatgaagatatagtagatgaatcgatagataaactgttaaggattgataatactttttggagaatgttcagcaaggttatgtttgaatcaaaggttaagaaaaggataatgttatatgatgtaaaatttctatcattagtaggttatatagggtttaagaattggtttatagaacagttgagatcagctgagttgcatgaggtaccttggattgtcaatgccgaaggtgatctggttgagatcaagtcaattaaaatctatttgcaactgatagagcagagtttatttttaagaataactgttttgaactatacagatatggcacatgctctcacaagattaatcagaaagaagttgatgtgtgataatgcactattaacttcaattccatccccaatggttaacttaactcaagttattgatcctacagaacaattagcttatttccctaagataacatttgaaaggctaaaaaattatgacactagttcaaattatgctaaaggaaagctaacaaggaattacatgatactgttgccatggcaacatgttaatagatataactttgtctttagttctactggatgtaaagttagtctaaaaacatgcattggaaaacttatgaaagatctaaaccctaaagttctgtactttattggagaaggggcaggaaattggatggccagaacagcatgtgaatatcctgacatcaaatttgtatacagaagtttaaaagatgaccttgatcatcattatcctttggaataccagagagtgataggagaattaagcaggataatagatagtggtgaagggctttcaatggaaacaacagatgcaactcaaaaaactcattgggatttgatacacagagtaagcaaagatgctttattaataactttatgtgatgcagaatttaaggacagagatgatttttttaagatggtaattctatggaggaaacatgtattatcatgcagaatttgcactacctatgggacagacctctatttattcgcaaagtatcatgctaaagactgcaatataaaattacctttttttgtgagatcagttgccacctttattatgcaaggtagtaaactgtcaggctcggaatgctacatactcttaacactaggccaccacaacaatttaccttgtcatggagaaatacaaaattctaagatgaaaatagcagcgtgtaatgatttttatgctgcaaaaaaacttgacaataaatcaattgaagccaactgtaaatcacttttatcagggctaagaataccgataaataagaaggaattaaatagacagagaaggttattaacactacaaagcaaccattcttctgtagcaacagttggaggtagcaaggtcatagagtctaaatggttaacaaacaaggcaaacacaataattgattggttagaacatattttaaattctccaaaaggtgaattaaattatgatttttttgaagcattagaaaatacttaccctaatatgattaaactaatagataatctagggaatgcagagattaaaaaactgatcaaagtaactggatatatgcttgtaagtaaaaaatgaaaaatgataaaaatgataaaataggtgacaacttcatactattccaaagtaatcatttgattatgcaattatgtaatagttaattaaaaactaaaaatcaaaagttaaaaactaataactgtcattaagtttattaaaaataagaaattataattggatgtatacggtttttttgccgt

SEQ ID NO: 11 - DNA, 15160 bp, synthetic construct
  misc_feature 1..15160: note = hMPV + exogenous DNA sequence + GFP
  source 1..15160: mol_type = other DNA
acgcgaaaaaaacgcgtataaattagattacaaaaaaatatgggacaagtgaaaatggtgagcaagggcgaggagctgttcaccggggtggtgcccatcctggtcgagctggacggcgacgtaaacggccacaagttcagcgtgtccggcgagggcgagggcgatgccacctacggcaagctgaccctgaagttcatctgcaccaccggcaagctgcccgtgccctggcccaccctcgtgaccaccctgacctacggcgtgcagtgcttcagccgctaccccgaccacatgaagcagcacgacttcttcaagtccgccatgcccgaaggctacgtccaggagcgcaccatcttcttcaaggacgacggcaactacaagacccgcgccgaggtgaagttcgagggcgacaccctggtgaaccgcatcgagctgaagggcatcgacttcaaggaggacggcaacatcctggggcacaagctggagtacaactacaacagccacaacgtctatatcatggccgacaagcagaagaacggcatcaaggtgaacttcaagatccgccacaacatcgaggacggcagcgtgcagctcgccgaccactaccagcagaacacccccatcggcgacggccccgtgctgctgcccgacaaccactacctgagcacccagtccgccctgagcaaagaccccaacgagaagcgcgatcacatggtcctgctggagttcgtgaccgccgccgggatcactctcggcatggacgagctgtacaagtaagttaattaaaaagtgggacaagtgaaaatgtctcttcaagggattcacctgagtgatctatcatacaagcatgctatattaaaagagtctcagtacacaataaaaagagatgtgggtacaacaactgcagtgacaccctcatcattgcaacaagaaataacgctgttgtgtggagaaattctgtatgctaaacatgctgattacaaatatgctgcagaaataggaatacaatatattagcacagctttaggatcagagagagtgcagcagattctgaggaactcaggcagtgaagtccaagtggtcttaaccagaacgtactctctggggaaagttaaaaacaataaaggagaagatttacagatgttagacatacacggggtagagaagagctgggtagaagagatagacaaagaagcaaggaaaacaatggcaaccttgcttaaggaatcatcaggtaatatcccacaaaatcagaggccctcagcaccagacacacccataatcttattatgtgtaggtgcattaatatttactaagctagcatcaaccatagaagtgggactagagaccacagtcagaagggctaaccgtgtactaagtgatgcactcaagagataccctagaatggacatcccaaaaattgccagatccttctatgacttatttgaacaaaaagtgtatcacagaagtttgttcattgagtatggcaaagcattaggctcatcatctacaggcagcaaagcagaaagtctatttgttaatatattcatgcaagcttatggagccggtcaaacaatgctaaggtggggggtcattgccaggtcatccaacaatataatgttaggacatgtatctgtccaagctgagttaaaacaggtcacagaagtctatgacttggtgcgagaaatgggccctgaatctggacttctacatttaaggcaaagcccaaaagctggactgttatcactagccaactgtcccaactttgcaagtgttgttctcggaaatgcctcaggcttaggcataatcggtatgtatcgtgggagagtaccaaacacagaattattttcagcagcagaaagttatgccaaaagtttgaaagagagcaataaaatcaatttctcttcattaggacttacagatgaagagaaagaggctgcagaacatttcttaaatgtgagtgacgacagtcaaaatgattatgagtaattaaaaaagtgggacaagtcaaaatgtctttccctgaaggaaaagatattcttttcatgggtaatgaagcagcaaaattagcagaagctttccagaaatcattaaggaaaccaagtcataaaagatctcaatctattataggagaaaaagtgaacactgtatcagaaacattggaattacctactatcagtagacctgcaaaaccaaccatactgtcagaaccaaagttagcatggactgataaaggtggggcaatcaaaactgaaataaagcaagcaatcaaagtcatggatcctattgaggaagaagagtctactgagaagaaggtgctgccctccagtgatgggaaaacccctgcagaaaagaaactgaaaccatcaactaacaccaaaaagaaagtttcgtttacaccaaatgaaccaggaaaatatacaaagttggaaaaagatgctctagatttgctctcagataatgaagaagaagatgcagaatcttcaatcttaacctttgaagaaagagatacttcatcgttaagcattgaggccagattggaatcaatagaggagaaattaagcatgatattagggctattaagaacactcaacattgctacagcaggacccacagcagcaagagatgggatcagagatgcaatgattggcgtaagagaggaattaatagcagacataataaaggaagccaaagggaaagcagcagaaatgatggaagaggaaatgagtcaacgatcaaaaataggaaacggtagtgtaaaactaacagagaaagcaaaagagcttaacaaaattgttgaagatgaaagcacaagtggagaatctgaagaagaagaagaaccaaaagacatacaagacaatagtcaagaagatgacatttaccagttaattatgtagtttaataaaaataaacaatgggacaagtaaaaatggagtcctacctagtagacacttatcaaggcattccttacacagcagctgttcaagttgatctaatagaaaaggacctgttacctgcaagcctaacaatatggttccctttgtttcaggccaacacaccaccagcagtgctgctcgatcagttgaaaaccctaacaataaccactctgtatgctgcatcacaaaatggtccaatactcaaagtgaatgcatcagcccaaggtgcagcaatgtctgtacttcccaaaaaatttgaagtcaatgcgactgtagcactcgatgaatatagcaaattggaatttgacaaactcacagtctgtgaagtaaaaacagtttacttaacaaccatgaaaccatacgggatggtatcaaaatttgtgagctcagccaaatcagttggcaaaaaaacacatgatctaatcgcactgtgtgattttatggatctagaaaagaacacacctgttacaataccagcattcatcaaatcagtttcaatcaaagagagtgagtcagctactgttgaagctgctataagcagtgaagcagaccaagctctaacacaggccaaaattgcaccttatgcgggattgattatgatcatgactatgaacaatcccaaaggcatattcaaaaagcttggagctgggactcaagttatagtagaactaggagcatatgtccaggctgaaagcataagtaaaatatgcaagacttggagccatcaagggacaagatatgtgttgaagtccagataacagccaagcaccttggccaagagctactaactctatctcatagattataaagtcaccattctagttatataaaaatcaagttagaacaagaattaaatcaatcaagaatgggacaaataaaaatgtcttggaaagtggtgatcattttttcattgttaataacacctcaacacggtcttaaagagagctatttagaagagtcatgtagcactataactgaaggatatctcagtgttctgaggacaggttggtataccaacgtttttacactggaggtaggtgatgtagagaaccttacatgtgctgatggacctagcttaataaaaacagaattagacctgaccaaaagtgcactaagagaactcagaacagtttctgctgatcaactggcaagagaggagcaaattgagaatcccagacaatctagatttgttctaggagcaatagcactcggtgttgcaacagcagctgcagttacagcaggtgttgcaattgccaaaaccatccggcttgaaagtgaagtaacagcaattaagaatgccctcaaaaagaccaatgaagcagtatctacattggggaatggagttcgagtgttggcaactgcagtgagggagctggaagattttgtgagcaagaatctaacacgtgcaatcaacaaaaacaagtgcgacattgctgacctgaaaatggccgttagcttcagtcaattcaacagaaggtttctaaatgttgtgcggcaattttcagacaatgctggaataacaccagcaatatccttggacttaatgacagatgctgaactagccagagctgtttccaacatgccaacatctgcaggacaaataaaactgatgttggagaaccgtgcaatggtaagaagaaaggggttcggaatcctgataggagtttacggaagctccgtaatttacatggtgcaactgccaatctttggagttatagacacgccttgctggatagtaaaagcggccccttcttgctcagaaaaaaagggaaactatgcttgccttttaagagaagatcaaggatggtattgtcagaatgcagggtcaactgtttactacccaaatgaaaaagactgcgaaacaagaggagaccatgtcttttgcgacacagcagcaggaatcaatgttgctgagcagtcaaaggagtgcaacatcaacatatccactactaattacccatgcaaagttagcacaggaagacaccctatcagtatggttgcactgtctcctcttggggctttggttgcttgctacaagggagtgagctgttccattggcagcaacagagtagggatcatcaagcaactgaacaaaggctgctcttatataaccaaccaagacgcagacacagtgacaatagacaacactgtataccagctaagcaaagttgagggcgaacagcatgttataaaaggaaggccagtgtcaagcagctttgatccagtcaaatttcctgaagatcaattcaatgttgcacttgaccaagttttcgaaagcattgagaacagtcaggccttggtggatcaatcaaacagaatcctaagcagtgcagagaaaggaaacactggcttcatcattgtaataattctaattgctgtccttggctctaccatgatcctagtgagtgtttttatcataataaagaaaacaaagaaacccacaggagcacctccagagctgagtggtgtcacaaacaatggcttcataccacataattagttaattaaaaataaaaacacatcagagtgggacaagtgaaaatggagttgctaatcctcaaagcaaatgcaattaccacaatcctcactgcagtcacattttgttttgcttctggtcaaaacatcactgaagaattttatcaatcaacatgcagtgcagttagcaaaggctatcttggagctctgagaactggttggtataccagtgttataactatagaattaagtaatatcaaggaaaataagtgtaatggaacagatgctaaggtaaaattgataaaacaagaattagataaatataaaaatgctgtaacagatttgcagttgctcatgcaaagcacaccagcaacaaacaatcgagccagaagagaactaccaaggtttatgaattatacactcaacaatgccaaaaaaaccaatgtaacattaagcaagaaaaggaaaagaagatttcttggttttttgttaggtgttggatctgcaatctgcagtggcgttgctgtatgcaaggtcctgcacctagaaggggaagtgaacaagatcaaaagtgctctactatccacaaacaaggctgtagtcagcttatcaaatggagtcagtgtcttaaccttcaaagtgttagacctcaaaaactatatagataaacaattgttacctattctcaacaagcaaagctgcagcataccaaatatagaaactgtgatagagttccaacaaaagaacaacagactactagagattaccagggaatttagtgttaatgcaggtgtaactacacctgtaagcacttacatgttaactaatagtgaattattgtcattaatcaatgatatgcctataacaaatgatcagaaaaagttaatgtccaacaatgttcaaatagttagacagcaaagttactctatcatgtgcataataaaagaggaagtcttagcatatgtagtacaattaccactatatggtgttatagatacaccctgttggaaactacacacatcccctctatgtacaaccaacacaaaagaagggtccaacatctgtttaacaagaactgacagaggatggtactgtgacaatgcaggatcagtatctttcttcccacaagctgaaacatgtaaagttcaatcaaatcgagtattttgtgacacaatgaacagtagaacattaccaagtgaagtaaatctctgcaatgttgacatattcaaccccaaatatgattgtaaaattatgacttcaaaaacagatgtaagcagctccgttatcacatctctaggagccattgtgtcatgctatggcaaaactaaatgtacagcatccaataaaaatcgtggaatcataaagacattttctaacgggtgcgattatgtatcaaataaaggggtggacactgtgtctgtaggtaacacattatattgtgtaaataagcaagaaggtcagagtctctatgtaaaaggtgaaccaataataaatttctatgacccattagtattcccctctgatgaatttgatgcatcaatatctcaagtcaacgagaagattaaccagagcctagcatttattcgtaaatccgatgaattattacataatgtaaatgccggtaaatccaccacaaatatcatgataactactataattatagtgattatagtaatattgttatcattaattgctgttggactgctcttatactgtaaggccagaagcacaccagtcacactaagcaaagatcaactgagtggtataaataatattgcatttagtaactaattaattaaaaataaagtaaattaaattaaaataaaataaaattaaaattaaaataaaataaaaataaaaatttgggacaaatcataatgtctcgcaaggctccatgcaaatatgaagtgcggggcaaatgcaatagaggaagtgagtgcaagtttaaccacaattactggagttggccagatagatacttactaataagatcaaattatttattaaatcaacttttaaggaacactgatagagctgatggcttatcaataatatcaggagcaggcagagaagataggacacaagattttgtcctaggttccaccaatgtggttcaaggttatattgatgataaccaaagcataacaaaagctgcagcctgttacagtctacataatataatcaaacaactacaagaagttgaagttaggcaggctagagataacaaaccatctgacagcaaacatgtggcacttcacaacttagtcctatcttatatggagatgagcaaaattcctgcatctttaatcaacaatctcaaaagactgccgagagagaaactgaaaaaattagcaaagcttataattgacttatcagcaggtgctgaaaatgactcttcatatgccttgcaagacagtgaaagcactaatcaagtgcagtgagcatggtcctgttttcattactatagaggttgattacatgatatggactcataaggacttaaaagaagctttatctaatgggatagtgaagtctcatactaacatttacaattgttatttagaaaacatagaaattatatatgtcaaggcttacttaagttagtaaaaacacatcagagtgggacaagtagttatggaggtgaaagtggagaacattcgaacaatagatatgctcaaagcaagagtgaaaaatcgtgtggcacgcagcaaatgctttaaaaatgcctctttgatcctaataggaataactacattgagtatagccctcaatatctatctaatcataaactatacaatgcaagaaaacacatccgaatcagaacatcacaccagctcatcacccatggaatccagcagggaaactccaacagtccctatggacaactcagacaccaatccaggctcacagtatccaactcaacagtccacagaaggctccacactctactttgcagcctcagcaagctcaccagagacagaaccaacatcaacaccagacacaacaagccgcccgcccttcgtcgacacacacacaacactaccaagtgcaagcagaacaaggacaagtccggcagtccacacaaaaaacaatccaaggacaagccccagaacacattccccaccatgggcaatgacaaggacggtccgtggaaccaccactctccgcacaagcagcacaagaaaaagaccgtccacagcatcagtccaacctgacagcagcgcaacaacccacaaacacgaagaagcaagcccagtgagcccgcaaacatctgcgagcacagcaagaccacaaaggaagggcatggaggccagcacatcaacaacatacaaccaaactagttaacaaaaaatacaaaataactctaagataaaccatatagacaccaacaattgagaagccaaaaggcaattcacaatctctccaaaaaggcaacaacaccatattagctccgcttaaatctccctggaaaaaacactcgcccatataccaactataccacaaccatcccaagaaaaaaagctgggtaaaacaacacccaagagacaaataacaatggatcctcttaatgaatccactgttaatgtctatcttcccgactcatatcttaaaggagtgatttcttttagtgagactaatgcaattggttcatgtctcttaaaaagaccttacctaaaaaatgacaacactgcaaaagttgccatagagaatcctgttatcgagcatgttagactcaaaaatgcaatcaattctaagatgaaaatatcagattacaggatagtagagccagtaaacatgcaacatgaaattatgaagaatgtacacagttgtgagctcacattattaaaacagtttttaacaaggagtaaaaatattagcactcttaaattaaatatgatatgtgattggctgcagttaaagtctacatcagatgatacctcaatcttaagttttatagatgtagaatttatacctagctgggtaagcaattggtttagtaattggtacaatctcaacaagttgattctggaattcaggaaagaagaagtaataagaactggttcaatcttgtgtaggtcattgggtaaattagtttttgttgtatcatcatacggatgtatagtcaagagcaacaaaagcaaaagagtgagcttcttcacatacaatcaactgttaacatggaaagatgtgatgttaagtagattcaatgcaaatttctgtatatgggtaagcaacagtctgaatgaaaatcaagaagggttagggttgagaagtaatctgcaaggcatattaactaataagctatatgaaactgtagattatatgcttagtttgtgttgcaatgaaggtttctcacttgtgaaagagtttgagggttttattatgagtgaaatccttaggattactgaacatgctcaattcagtactagatttagaaatactttattaaatggattaactgatcaattgacaaaattaaaaaataaaaacagactcagagttcatggtaccgtgttagaaaataatgattatccaatgtatgaagttgtacttaaattattaggagatactttgagatgtattaaattattaatcaataaaaacttagagaatgctgctgaattatactatatatttagaatattcggtcacccaatggtagatgaaagagatgcaatggatgctgtcaaattaaacaatgaaatcacaaaaatcctcaggttggagagcttgacagaactaagaggggcattcatattaaggattatcaaaggatttgtagacaacaacaaaagatggccgaaaattaaaaacttaaaagtgcttagtaaaagatggactatgtacttcaaagcaaaaagttaccctagtcaacttgaattaagtgaacaagattttttagagcttgctgcaatacagtttgaacaagagttttctgttcctgaaaaaaccaaccttgagatggtattaaatgataaagctatatcacctcctaaaagattaatttggtctgtgtacccaaaaaattacttacctgagacaataaaaaatcgatatctagaagagactttcaatgcaagtgatagtctcaaaacaagaagagtactagagtactatttgaaagataataaattcgaccaaaaagaacttaaaagttatgtggttaaacaagaatatttaaatgataaggatcatattgtctcgctaactggaaaagaaagagaattaagtgtaggtagaatgtttgctatgcaaccaggaaaacagcgacaaatacaaatattggctgaaaaattgttagctgataatattgtaccctttttcccagaaactttaacaaagtatggtgatctagatcttcagagaataatggaaatcaaatcagaactttcttctattaaaaccagaagaaatgatagttataataattacattgcaagagcatccatagtaacagatttaagtaagttcaaccaagcctttaggtatgaaactacagcgatctgtgcggatgtagcagatgaactacatggaacacaaagcctattctgttggttacatcttatcgttcctatgactacaatgatatgtgcctatagacatgcaccaccagaaacaaaaggtgaatatgatatagataagatagaagagcaaagtggtttatatagatatcatatgggtggtattgaaggatggtgtcaaaaactctggacaatggaagctatatctttattagatgttgtatctgtaaagacacgatgtcaaatgacatctttattaaacggtgacaaccaatcaatagatgtaagtaaaccagttaagttatctgagggtttagatgaagtgaaagcagattatagcttggctgtaaaaatgctaaaagaaataagagatgcatacagaaatataggccataaacttaaagaaggggaaacatatatatcaagagatcttcagtttataagtaaggtgattcaatctgaaggagtaatgcatcctacccctataaaaaagatcttaagagtgggaccatggataaacacaatattagatgacattaaaaccagtgcagagtcaatagggagtctatgtcaggaattagaatttaggggggaaagcataatagttagtctgatattaaggaatttttggctgtataatttatacatgcatgaatcaaagcaacaccccctagcagggaagcagttattcaaacaactaaataaaacattaacatcagtgcagagattttttgaaattaaaaaggaaaatgaagtagtagatctatggatgaacataccaatgcagtttggaggaggagatccagtagtcttctatagatctttctatagaaggacccctgattttttaactgaagcaatcagtcatgtagatattctgttaaaaatatcagccaacataagaaatgaagcgaaaataagtttcttcaaagccttactgtcaatagaaaaaaatgaacgtgctacactgacaacactaatgagagaccctcaagctgtgggctcagagcgacaagcaaaagtaacaagtgatatcaatagaacagcagttaccagcatcttaagtctttctccaaatcaacttttcagcgatagtgctatacactacagtagaaatgaagaagaggtcggaatcattgctgacaacataacacctgtttatcctcatggactgagagttttgtatgaatcattaccttttcataaagctgaaaaagttgtaaatatgatatcaggaacaaaatccataaccaacttattacagagaacatctgctattaatggtgaagatattgacagagctgtatccatgatgctggagaacctaggattattatctagaatattgtcagtagttgttgatagtatagaaattccaaccaaatctaatggtaggctgatatgttgtcagatatctagaaccctaagggagacatcatggaataatatggaaatagttggagtaacatcccctagcatcactacatgcatggatgtcatatatgcaactagctctcatttgaaagggataatcattgaaaagttcagcactgacagaactacaagaggtcaaagaggtccaaagagcccttgggtaggatcgagcacgcaagagaaaaaattagttcctgtttataacagacaaattctttcaaaacaacaaagagaacagctagaagcaattggaaaaatgagatgggtatataaagggacaccaggtttaagacgattactcaataagatttgtcttggaagtttaggcattagttacaaatgtgtaaaacctttattacctaggtttatgagtgtaaatttcctacacaggttatctgtcagtagtagacctatggaattcccagcatcagttccagcttatagaacaacaaattaccattttgacactagtcctattaatcaagcactaagtgaaagatttgggaatgaagatattaatttggtcttccaaaatgcaatcagctgtggaattagcataatgagtgtagtagaacaattaactggtaggagtccaaaacagttagttttaataccccaattagaagaaatagacattatgccaccaccagtgtttcaagggaaattcaattataagctagtagataagataacttctgaccaacatatcttcagtccagacaaaatagatatgttaacactggggaaaatgctcatgccaactataaaaggtcagaaaacagatcagttcttgaacaagagagagaattatttccatgggaacaatcttattgagtctttgtcagcagcgttagcatgtcattggtgtgggatattaacagagcaatgtatagaaaataatattttcaagaaagactggggtgacgggttcatatcggatcatgcttttatggacttcaaaatattcctatgtgtctttaaaactaaacttttatgtagttggggatcccaagggaaaaacattaaagatgaagatatagtagatgaatcgatagataaactgttaaggattgataatactttttggagaatgttcagcaaggttatgtttgaatcaaaggttaagaaaaggataatgttatatgatgtaaaatttctatcattagtaggttatatagggtttaagaattggtttatagaacagttgagatcagctgagttgcatgaggtaccttggattgtcaatgccgaaggtgatctggttgagatcaagtcaattaaaatctatttgcaactgatagagcagagtttatttttaagaataactgttttgaactatacagatatggcacatgctctcacaagattaatcagaaagaagttgatgtgtgataatgcactattaacttcaattccatccccaatggttaacttaactcaagttattgatcctacagaacaattagcttatttccctaagataacatttgaaaggctaaaaaattatgacactagttcaaattatgctaaaggaaagctaacaaggaattacatgatactgttgccatggcaacatgttaatagatataactttgtctttagttctactggatgtaaagttagtctaaaaacatgcattggaaaacttatgaaagatctaaaccctaaagttctgtactttattggagaaggggcaggaaattggatggccagaacagcatgtgaatatcctgacatcaaatttgtatacagaagtttaaaagatgaccttgatcatcattatcctttggaataccagagagtgataggagaattaagcaggataatagatagtggtgaagggctttcaatggaaacaacagatgcaactcaaaaaactcattgggatttgatacacagagtaagcaaagatgctttattaataactttatgtgatgcagaatttaaggacagagatgatttttttaagatggtaattctatggaggaaacatgtattatcatgcagaatttgcactacctatgggacagacctctatttattcgcaaagtatcatgctaaagactgcaatataaaattacctttttttgtgagatcagttgccacctttattatgcaaggtagtaaactgtcaggctcggaatgctacatactcttaacactaggccaccacaacaatttaccttgtcatggagaaatacaaaattctaagatgaaaatagcagcgtgtaatgatttttatgctgcaaaaaaacttgacaataaatcaattgaagccaactgtaaatcacttttatcagggctaagaataccgataaataagaaggaattaaatagacagagaaggttattaacactacaaagcaaccattcttctgtagcaacagttggaggtagcaaggtcatagagtctaaatggttaacaaacaaggcaaacacaataattgattggttagaacatattttaaattctccaaaaggtgaattaaattatgatttttttgaagcattagaaaatacttaccctaatatgattaaactaatagataatctagggaatgcagagattaaaaaactgatcaaagtaactggatatatgcttgtaagtaaaaaatgaaaaatgataaaaatgataaaataggtgacaacttcatactattccaaagtaatcatttgattatgcaattatgtaatagttaattaaaaactaaaaatcaaaagttaaaaactaataactgtcattaagtttattaaaaataagaaattataattggatgtatacggtttttttgccgt

SEQ ID NO: 12 - DNA, 15151 bp, synthetic construct
  misc_feature 1..15151: note = hMPV + exogenous DNA sequence + GFP
  source 1..15151: mol_type = other DNA
acgcgaaaaaaacgcgtataaattagattacaaaaaaatatgggacaagtgaaaatggtgagcaagggcgaggagctgttcaccggggtggtgcccatcctggtcgagctggacggcgacgtaaacggccacaagttcagcgtgtccggcgagggcgagggcgatgccacctacggcaagctgaccctgaagttcatctgcaccaccggcaagctgcccgtgccctggcccaccctcgtgaccaccctgacctacggcgtgcagtgcttcagccgctaccccgaccacatgaagcagcacgacttcttcaagtccgccatgcccgaaggctacgtccaggagcgcaccatcttcttcaaggacgacggcaactacaagacccgcgccgaggtgaagttcgagggcgacaccctggtgaaccgcatcgagctgaagggcatcgacttcaaggaggacggcaacatcctggggcacaagctggagtacaactacaacagccacaacgtctatatcatggccgacaagcagaagaacggcatcaaggtgaacttcaagatccgccacaacatcgaggacggcagcgtgcagctcgccgaccactaccagcagaacacccccatcggcgacggccccgtgctgctgcccgacaaccactacctgagcacccagtccgccctgagcaaagaccccaacgagaagcgcgatcacatggtcctgctggagttcgtgaccgccgccgggatcactctcggcatggacgagctgtacaagtaagttaattaaaaagtgggacaagtgaaaatgtctcttcaagggattcacctgagtgatctatcatacaagcatgctatattaaaagagtctcagtacacaataaaaagagatgtgggtacaacaactgcagtgacaccctcatcattgcaacaagaaataacgctgttgtgtggagaaattctgtatgctaaacatgctgattacaaatatgctgcagaaataggaatacaatatattagcacagctttaggatcagagagagtgcagcagattctgaggaactcaggcagtgaagtccaagtggtcttaaccagaacgtactctctggggaaagttaaaaacaataaaggagaagatttacagatgttagacatacacggggtagagaagagctgggtagaagagatagacaaagaagcaaggaaaacaatggcaaccttgcttaaggaatcatcaggtaatatcccacaaaatcagaggccctcagcaccagacacacccataatcttattatgtgtaggtgcattaatatttactaagctagcatcaaccatagaagtgggactagagaccacagtcagaagggctaaccgtgtactaagtgatgcactcaagagataccctagaatggacatcccaaaaattgccagatccttctatgacttatttgaacaaaaagtgtatcacagaagtttgttcattgagtatggcaaagcattaggctcatcatctacaggcagcaaagcagaaagtctatttgttaatatattcatgcaagcttatggagccggtcaaacaatgctaaggtggggggtcattgccaggtcatccaacaatataatgttaggacatgtatctgtccaagctgagttaaaacaggtcacagaagtctatgacttggtgcgagaaatgggccctgaatctggacttctacatttaaggcaaagcccaaaagctggactgttatcactagccaactgtcccaactttgcaagtgttgttctcggaaatgcctcaggcttaggcataatcggtatgtatcgtgggagagtaccaaacacagaattattttcagcagcagaaagttatgccaaaagtttgaaagagagcaataaaatcaatttctcttcattaggacttacagatgaagagaaagaggctgcagaacatttcttaaatgtgagtgacgacagtcaaaatgattatgagtaattaaaaaagtgggacaagtcaaaatgtctttccctgaaggaaaagatattcttttcatgggtaatgaagcagcaaaattagcagaagctttccagaaatcattaaggaaaccaagtcataaaagatctcaatctattataggagaaaaagtgaacactgtatcagaaacattggaattacctactatcagtagacctgcaaaaccaaccatactgtcagaaccaaagttagcatggactgataaaggtggggcaatcaaaactgaaataaagcaagcaatcaaagtcatggatcctattgaggaagaagagtctactgagaagaaggtgctgccctccagtgatgggaaaacccctgcagaaaagaaactgaaaccatcaactaacaccaaaaagaaagtttcgtttacaccaaatgaaccaggaaaatatacaaagttggaaaaagatgctctagatttgctctcagataatgaagaagaagatgcagaatcttcaatcttaacctttgaagaaagagatacttcatcgttaagcattgaggccagattggaatcaatagaggagaaattaagcatgatattagggctattaagaacactcaacattgctacagcaggacccacagcagcaagagatgggatcagagatgcaatgattggcgtaagagaggaattaatagcagacataataaaggaagccaaagggaaagcagcagaaatgatggaagaggaaatgagtcaacgatcaaaaataggaaacggtagtgtaaaactaacagagaaagcaaaagagcttaacaaaattgttgaagatgaaagcacaagtggagaatctgaagaagaagaagaaccaaaagacatacaagacaatagtcaagaagatgacatttaccagttaattatgtagtttaataaaaataaacaatgggacaagtaaaaatggagtcctacctagtagacacttatcaaggcattccttacacagcagctgttcaagttgatctaatagaaaaggacctgttacctgcaagcctaacaatatggttccctttgtttcaggccaacacaccaccagcagtgctgctcgatcagttgaaaaccctaacaataaccactctgtatgctgcatcacaaaatggtccaatactcaaagtgaatgcatcagcccaaggtgcagcaatgtctgtacttcccaaaaaatttgaagtcaatgcgactgtagcactcgatgaatatagcaaattggaatttgacaaactcacagtctgtgaagtaaaaacagtttacttaacaaccatgaaaccatacgggatggtatcaaaatttgtgagctcagccaaatcagttggcaaaaaaacacatgatctaatcgcactgtgtgattttatggatctagaaaagaacacacctgttacaataccagcattcatcaaatcagtttcaatcaaagagagtgagtcagctactgttgaagctgctataagcagtgaagcagaccaagctctaacacaggccaaaattgcaccttatgcgggattgattatgatcatgactatgaacaatcccaaaggcatattcaaaaagcttggagctgggactcaagttatagtagaactaggagcatatgtccaggctgaaagcataagtaaaatatgcaagacttggagccatcaagggacaagatatgtgttgaagtccagataacagccaagcaccttggccaagagctactaactctatctcatagattataaagtcaccattctagttatataaaaatcaagttagaacaagaattaaatcaatcaagaatgggacaaataaaaatgtcttggaaagtggtgatcattttttcattgttaataacacctcaacacggtcttaaagagagctatttagaagagtcatgtagcactataactgaaggatatctcagtgttctgaggacaggttggtataccaacgtttttacactggaggtaggtgatgtagagaaccttacatgtgctgatggacctagcttaataaaaacagaattagacctgaccaaaagtgcactaagagaactcagaacagtttctgctgatcaactggcaagagaggagcaaattgagaatcccagacaatctagatttgttctaggagcaatagcactcggtgttgcaacagcagctgcagttacagcaggtgttgcaattgccaaaaccatccggcttgaaagtgaagtaacagcaattaagaatgccctcaaaaagaccaatgaagcagtatctacattggggaatggagttcgagtgttggcaactgcagtgagggagctggaagattttgtgagcaagaatctaacacgtgcaatcaacaaaaacaagtgcgacattgctgacctgaaaatggccgttagcttcagtcaattcaacagaaggtttctaaatgttgtgcggcaattttcagacaatgctggaataacaccagcaatatccttggacttaatgacagatgctgaactagccagagctgtttccaacatgccaacatctgcaggacaaataaaactgatgttggagaaccgtgcaatggtaagaagaaaggggttcggaatcctgataggagtttacggaagctccgtaatttacatggtgcaactgccaatctttggagttatagacacgccttgctggatagtaaaagcggccccttcttgctcagaaaaaaagggaaactatgcttgccttttaagagaagatcaaggatggtattgtcagaatgcagggtcaactgtttactacccaaatgaaaaagactgcgaaacaagaggagaccatgtcttttgcgacacagcagcaggaatcaatgttgctgagcagtcaaaggagtgcaacatcaacatatccactactaattacccatgcaaagttagcacaggaagacaccctatcagtatggttgcactgtctcctcttggggctttggttgcttgctacaagggagtgagctgttccattggcagcaacagagtagggatcatcaagcaactgaacaaaggctgctcttatataaccaaccaagacgcagacacagtgacaatagacaacactgtataccagctaagcaaagttgagggcgaacagcatgttataaaaggaaggccagtgtcaagcagctttgatccagtcaaatttcctgaagatcaattcaatgttgcacttgaccaagttttcgaaagcattgagaacagtcaggccttggtggatcaatcaaacagaatcctaagcagtgcagagaaaggaaacactggcttcatcattgtaataattctaattgctgtccttggctctaccatgatcctagtgagtgtttttatcataataaagaaaacaaagaaacccacaggagcacctccagagctgagtggtgtcacaaacaatggcttcataccacataattagttaattaaaaataaaaacacatcagagtgggacaagtgaaaatggagttgctaatcctcaaagcaaatgcaattaccacaatcctcactgcagtcacattttgttttgcttctggtcaaaacatcactgaagaattttatcaatcaacatgcagtgcagttagcaaaggctatcttggagctctgagaactggttggtataccagtgttataactatagaattaagtaatatcaaggaaaataagtgtaatggaacagatgctaaggtaaaattgataaaacaagaattagataaatataaaaatgctgtaacagatttgcagttgctcatgcaaagcacaccagcaacaaacaatcgagccagaagagaactaccaaggtttatgaattatacactcaacaatgccaaaaaaaccaatgtaacattaagcaagaaaaggaaaagaagatttcttggttttttgttaggtgttggatctgcaatctgcagtggcgttgctgtatgcaaggtcctgcacctagaaggggaagtgaacaagatcaaaagtgctctactatccacaaacaaggctgtagtcagcttatcaaatggagtcagtgtcttaaccttcaaagtgttagacctcaaaaactatatagataaacaattgttacctattctcaacaagcaaagctgcagcataccaaatatagaaactgtgatagagttccaacaaaagaacaacagactactagagattaccagggaatttagtgttaatgcaggtgtaactacacctgtaagcacttacatgttaactaatagtgaattattgtcattaatcaatgatatgcctataacaaatgatcagaaaaagttaatgtccaacaatgttcaaatagttagacagcaaagttactctatcatgtgcataataaaagaggaagtcttagcatatgtagtacaattaccactatatggtgttatagatacaccctgttggaaactacacacatcccctctatgtacaaccaacacaaaagaagggtccaacatctgtttaacaagaactgacagaggatggtactgtgacaatgcaggatcagtatctttcttcccacaagctgaaacatgtaaagttcaatcaaatcgagtattttgtgacacaatgaacagtagaacattaccaagtgaagtaaatctctgcaatgttgacatattcaaccccaaatatgattgtaaaattatgacttcaaaaacagatgtaagcagctccgttatcacatctctaggagccattgtgtcatgctatggcaaaactaaatgtacagcatccaataaaaatcgtggaatcataaagacattttctaacgggtgcgattatgtatcaaataaaggggtggacactgtgtctgtaggtaacacattatattgtgtaaataagcaagaaggtcagagtctctatgtaaaaggtgaaccaataataaatttctatgacccattagtattcccctctgatgaatttgatgcatcaatatctcaagtcaacgagaagattaaccagagcctagcatttattcgtaaatccgatgaattattaagtagtgctgaaaagggtaatacgggcttcattatcgtcattatattgatcgccgtgttggggagcacgatgattttggtctccgtgttcattattatcaaaaagactaaaaagccgacgggtgcgccacctgaactctccggcgtgactaataacgggttcattcctcacaactaattaattaaaaataaagtaaattaaattaaaataaaataaaattaaaattaaaataaaataaaaataaaaatttgggacaaatcataatgtctcgcaaggctccatgcaaatatgaagtgcggggcaaatgcaatagaggaagtgagtgcaagtttaaccacaattactggagttggccagatagatacttactaataagatcaaattatttattaaatcaacttttaaggaacactgatagagctgatggcttatcaataatatcaggagcaggcagagaagataggacacaagattttgtcctaggttccaccaatgtggttcaaggttatattgatgataaccaaagcataacaaaagctgcagcctgttacagtctacataatataatcaaacaactacaagaagttgaagttaggcaggctagagataacaaaccatctgacagcaaacatgtggcacttcacaacttagtcctatcttatatggagatgagcaaaattcctgcatctttaatcaacaatctcaaaagactgccgagagagaaactgaaaaaattagcaaagcttataattgacttatcagcaggtgctgaaaatgactcttcatatgccttgcaagacagtgaaagcactaatcaagtgcagtgagcatggtcctgttttcattactatagaggttgattacatgatatggactcataaggacttaaaagaagctttatctaatgggatagtgaagtctcatactaacatttacaattgttatttagaaaacatagaaattatatatgtcaaggcttacttaagttagtaaaaacacatcagagtgggacaagtagttatggaggtgaaagtggagaacattcgaacaatagatatgctcaaagcaagagtgaaaaatcgtgtggcacgcagcaaatgctttaaaaatgcctctttgatcctaataggaataactacattgagtatagccctcaatatctatctaatcataaactatacaatgcaagaaaacacatccgaatcagaacatcacaccagctcatcacccatggaatccagcagggaaactccaacagtccctatggacaactcagacaccaatccaggctcacagtatccaactcaacagtccacagaaggctccacactctactttgcagcctcagcaagctcaccagagacagaaccaacatcaacaccagacacaacaagccgcccgcccttcgtcgacacacacacaacactaccaagtgcaagcagaacaaggacaagtccggcagtccacacaaaaaacaatccaaggacaagccccagaacacattccccaccatgggcaatgacaaggacggtccgtggaaccaccactctccgcacaagcagcacaagaaaaagaccgtccacagcatcagtccaacctgacagcagcgcaacaacccacaaacacgaagaagcaagcccagtgagcccgcaaacatctgcgagcacagcaagaccacaaaggaagggcatggaggccagcacatcaacaacatacaaccaaactagttaacaaaaaatacaaaataactctaagataaaccatatagacaccaacaattgagaagccaaaaggcaattcacaatctctccaaaaaggcaacaacaccatattagctccgcttaaatctccctggaaaaaacactcgcccatataccaactataccacaaccatcccaagaaaaaaagctgggtaaaacaacacccaagagacaaataacaatggatcctcttaatgaatccactgttaatgtctatcttcccgactcatatcttaaaggagtgatttcttttagtgagactaatgcaattggttcatgtctcttaaaaagaccttacctaaaaaatgacaacactgcaaaagttgccatagagaatcctgttatcgagcatgttagactcaaaaatgcaatcaattctaagatgaaaatatcagattacaggatagtagagccagtaaacatgcaacatgaaattatgaagaatgtacacagttgtgagctcacattattaaaacagtttttaacaaggagtaaaaatattagcactcttaaattaaatatgatatgtgattggctgcagttaaagtctacatcagatgatacctcaatcttaagttttatagatgtagaatttatacctagctgggtaagcaattggtttagtaattggtacaatctcaacaagttgattctggaattcaggaaagaagaagtaataagaactggttcaatcttgtgtaggtcattgggtaaattagtttttgttgtatcatcatacggatgtatagtcaagagcaacaaaagcaaaagagtgagcttcttcacatacaatcaactgttaacatggaaagatgtgatgttaagtagattcaatgcaaatttctgtatatgggtaagcaacagtctgaatgaaaatcaagaagggttagggttgagaagtaatctgcaaggcatattaactaataagctatatgaaactgtagattatatgcttagtttgtgttgcaatgaaggtttctcacttgtgaaagagtttgagggttttattatgagtgaaatccttaggattactgaacatgctcaattcagtactagatttagaaatactttattaaatggattaactgatcaattgacaaaattaaaaaataaaaacagactcagagttcatggtaccgtgttagaaaataatgattatccaatgtatgaagttgtacttaaattattaggagatactttgagatgtattaaattattaatcaataaaaacttagagaatgctgctgaattatactatatatttagaatattcggtcacccaatggtagatgaaagagatgcaatggatgctgtcaaattaaacaatgaaatcacaaaaatcctcaggttggagagcttgacagaactaagaggggcattcatattaaggattatcaaaggatttgtagacaacaacaaaagatggccgaaaattaaaaacttaaaagtgcttagtaaaagatggactatgtacttcaaagcaaaaagttaccctagtcaacttgaattaagtgaacaagattttttagagcttgctgcaatacagtttgaacaagagttttctgttcctgaaaaaaccaaccttgagatggtattaaatgataaagctatatcacctcctaaaagattaatttggtctgtgtacccaaaaaattacttacctgagacaataaaaaatcgatatctagaagagactttcaatgcaagtgatagtctcaaaacaagaagagtactagagtactatttgaaagataataaattcgaccaaaaagaacttaaaagttatgtggttaaacaagaatatttaaatgataaggatcatattgtctcgctaactggaaaagaaagagaattaagtgtaggtagaatgtttgctatgcaaccaggaaaacagcgacaaatacaaatattggctgaaaaattgttagctgataatattgtaccctttttcccagaaactttaacaaagtatggtgatctagatcttcagagaataatggaaatcaaatcagaactttcttctattaaaaccagaagaaatgatagttataataattacattgcaagagcatccatagtaacagatttaagtaagttcaaccaagcctttaggtatgaaactacagcgatctgtgcggatgtagcagatgaactacatggaacacaaagcctattctgttggttacatcttatcgttcctatgactacaatgatatgtgcctatagacatgcaccaccagaaacaaaaggtgaatatgatatagataagatagaagagcaaagtggtttatatagatatcatatgggtggtattgaaggatggtgtcaaaaactctggacaatggaagctatatctttattagatgttgtatctgtaaagacacgatgtcaaatgacatctttattaaacggtgacaaccaatcaatagatgtaagtaaaccagttaagttatctgagggtttagatgaagtgaaagcagattatagcttggctgtaaaaatgctaaaagaaataagagatgcatacagaaatataggccataaacttaaagaaggggaaacatatatatcaagagatcttcagtttataagtaaggtgattcaatctgaaggagtaatgcatcctacccctataaaaaagatcttaagagtgggaccatggataaacacaatattagatgacattaaaaccagtgcagagtcaatagggagtctatgtcaggaattagaatttaggggggaaagcataatagttagtctgatattaaggaatttttggctgtataatttatacatgcatgaatcaaagcaacaccccctagcagggaagcagttattcaaacaactaaataaaacattaacatcagtgcagagattttttgaaattaaaaaggaaaatgaagtagtagatctatggatgaacataccaatgcagtttggaggaggagatccagtagtcttctatagatctttctatagaaggacccctgattttttaactgaagcaatcagtcatgtagatattctgttaaaaatatcagccaacataagaaatgaagcgaaaataagtttcttcaaagccttactgtcaatagaaaaaaatgaacgtgctacactgacaacactaatgagagaccctcaagctgtgggctcagagcgacaagcaaaagtaacaagtgatatcaatagaacagcagttaccagcatcttaagtctttctccaaatcaacttttcagcgatagtgctatacactacagtagaaatgaagaagaggtcggaatcattgctgacaacataacacctgtttatcctcatggactgagagttttgtatgaatcattaccttttcataaagctgaaaaagttgtaaatatgatatcaggaacaaaatccataaccaacttattacagagaacatctgctattaatggtgaagatattgacagagctgtatccatgatgctggagaacctaggattattatctagaatattgtcagtagttgttgatagtatagaaattccaaccaaatctaatggtaggctgatatgttgtcagatatctagaaccctaagggagacatcatggaataatatggaaatagttggagtaacatcccctagcatcactacatgcatggatgtcatatatgcaactagctctcatttgaaagggataatcattgaaaagttcagcactgacagaactacaagaggtcaaagaggtccaaagagcccttgggtaggatcgagcacgcaagagaaaaaattagttcctgtttataacagacaaattctttcaaaacaacaaagagaacagctagaagcaattggaaaaatgagatgggtatataaagggacaccaggtttaagacgattactcaataagatttgtcttggaagtttaggcattagttacaaatgtgtaaaacctttattacctaggtttatgagtgtaaatttcctacacaggttatctgtcagtagtagacctatggaattcccagcatcagttccagcttatagaacaacaaattaccattttgacactagtcctattaatcaagcactaagtgaaagatttgggaatgaagatattaatttggtcttccaaaatgcaatcagctgtggaattagcataatgagtgtagtagaacaattaactggtaggagtccaaaacagttagttttaataccccaattagaagaaatagacattatgccaccaccagtgtttcaagggaaattcaattataagctagtagataagataacttctgaccaacatatcttcagtccagacaaaatagatatgttaacactggggaaaatgctcatgccaactataaaaggtcagaaaacagatcagttcttgaacaagagagagaattatttccatgggaacaatcttattgagtctttgtcagcagcgttagcatgtcattggtgtgggatattaacagagcaatgtatagaaaataatattttcaagaaagactggggtgacgggttcatatcggatcatgcttttatggacttcaaaatattcctatgtgtctttaaaactaaacttttatgtagttggggatcccaagggaaaaacattaaagatgaagatatagtagatgaatcgatagataaactgttaaggattgataatactttttggagaatgttcagcaaggttatgtttgaatcaaaggttaagaaaaggataatgttatatgatgtaaaatttctatcattagtaggttatatagggtttaagaattggtttatagaacagttgagatcagctgagttgcatgaggtaccttggattgtcaatgccgaaggtgatctggttgagatcaagtcaattaaaatctatttgcaactgatagagcagagtttatttttaagaataactgttttgaactatacagatatggcacatgctctcacaagattaatcagaaagaagttgatgtgtgataatgcactattaacttcaattccatccccaatggttaacttaactcaagttattgatcctacagaacaattagcttatttccctaagataacatttgaaaggctaaaaaattatgacactagttcaaattatgctaaaggaaagctaacaaggaattacatgatactgttgccatggcaacatgttaatagatataactttgtctttagttctactggatgtaaagttagtctaaaaacatgcattggaaaacttatgaaagatctaaaccctaaagttctgtactttattggagaaggggcaggaaattggatggccagaacagcatgtgaatatcctgacatcaaatttgtatacagaagtttaaaagatgaccttgatcatcattatcctttggaataccagagagtgataggagaattaagcaggataatagatagtggtgaagggctttcaatggaaacaacagatgcaactcaaaaaactcattgggatttgatacacagagtaagcaaagatgctttattaataactttatgtgatgcagaatttaaggacagagatgatttttttaagatggtaattctatggaggaaacatgtattatcatgcagaatttgcactacctatgggacagacctctatttattcgcaaagtatcatgctaaagactgcaatataaaattacctttttttgtgagatcagttgccacctttattatgcaaggtagtaaactgtcaggctcggaatgctacatactcttaacactaggccaccacaacaatttaccttgtcatggagaaatacaaaattctaagatgaaaatagcagcgtgtaatgatttttatgctgcaaaaaaacttgacaataaatcaattgaagccaactgtaaatcacttttatcagggctaagaataccgataaataagaaggaattaaatagacagagaaggttattaacactacaaagcaaccattcttctgtagcaacagttggaggtagcaaggtcatagagtctaaatggttaacaaacaaggcaaacacaataattgattggttagaacatattttaaattctccaaaaggtgaattaaattatgatttttttgaagcattagaaaatacttaccctaatatgattaaactaatagataatctagggaatgcagagattaaaaaactgatcaaagtaactggatatatgcttgtaagtaaaaaatgaaaaatgataaaaatgataaaataggtgacaacttcatactattccaaagtaatcatttgattatgcaattatgtaatagttaattaaaaactaaaaatcaaaagttaaaaactaataactgtcattaagtttattaaaaataagaaattataattggatgtatacggtttttttgccgt

SEQ ID NO: 13 - DNA, 13392 bp, synthetic construct
  misc_feature 1..13392: note = hMPV + GFP
  source 1..13392: mol_type = other DNA
gcgaaaaaaacgcgtataaattagattacaaaaaaatatgggacaagtgaaaatggtgagcaagggcgaggagctgttcaccggggtggtgcccatcctggtcgagctggacggcgacgtaaacggccacaagttcagcgtgtccggcgagggcgagggcgatgccacctacggcaagctgaccctgaagttcatctgcaccaccggcaagctgcccgtgccctggcccaccctcgtgaccaccctgacctacggcgtgcagtgcttcagccgctaccccgaccacatgaagcagcacgacttcttcaagtccgccatgcccgaaggctacgtccaggagcgcaccatcttcttcaaggacgacggcaactacaagacccgcgccgaggtgaagttcgagggcgacaccctggtgaaccgcatcgagctgaagggcatcgacttcaaggaggacggcaacatcctggggcacaagctggagtacaactacaacagccacaacgtctatatcatggccgacaagcagaagaacggcatcaaggtgaacttcaagatccgccacaacatcgaggacggcagcgtgcagctcgccgaccactaccagcagaacacccccatcggcgacggccccgtgctgctgcccgacaaccactacctgagcacccagtccgccctgagcaaagaccccaacgagaagcgcgatcacatggtcctgctggagttcgtgaccgccgccgggatcactctcggcatggacgagctgtacaagtaagttaattaaaaagtgggacaagtgaaaatgtctcttcaagggattcacctgagtgatctatcatacaagcatgctatattaaaagagtctcagtacacaataaaaagagatgtgggtacaacaactgcagtgacaccctcatcattgcaacaagaaataacgctgttgtgtggagaaattctgtatgctaaacatgctgattacaaatatgctgcagaaataggaatacaatatattagcacagctttaggatcagagagagtgcagcagattctgaggaactcaggcagtgaagtccaagtggtcttaaccagaacgtactctctggggaaagttaaaaacaataaaggagaagatttacagatgttagacatacacggggtagagaagagctgggtagaagagatagacaaagaagcaaggaaaacaatggcaaccttgcttaaggaatcatcaggtaatatcccacaaaatcagaggccctcagcaccagacacacccataatcttattatgtgtaggtgcattaatatttactaagctagcatcaaccatagaagtgggactagagaccacagtcagaagggctaaccgtgtactaagtgatgcactcaagagataccctagaatggacatcccaaaaattgccagatccttctatgacttatttgaacaaaaagtgtatcacagaagtttgttcattgagtatggcaaagcattaggctcatcatctacaggcagcaaagcagaaagtctatttgttaatatattcatgcaagcttatggagccggtcaaacaatgctaaggtggggggtcattgccaggtcatccaacaatataatgttaggacatgtatctgtccaagctgagttaaaacaggtcacagaagtctatgacttggtgcgagaaatgggccctgaatctggacttctacatttaaggcaaagcccaaaagctggactgttatcactagccaactgtcccaactttgcaagtgttgttctcggaaatgcctcaggcttaggcataatcggtatgtatcgtgggagagtaccaaacacagaattattttcagcagcagaaagttatgccaaaagtttgaaagagagcaataaaatcaatttctcttcattaggacttacagatgaagagaaagaggctgcagaacatttcttaaatgtgagtgacgacagtcaaaatgattatgagtaattaaaaaagtgggacaagtcaaaatgtctttccctgaaggaaaagatattcttttcatgggtaatgaagcagcaaaattagcagaagctttccagaaatcattaaggaaaccaagtcataaaagatctcaatctattataggagaaaaagtgaacactgtatcagaaacattggaattacctactatcagtagacctgcaaaaccaaccatactgtcagaaccaaagttagcatggactgataaaggtggggcaatcaaaactgaaataaagcaagcaatcaaagtcatggatcctattgaggaagaagagtctactgagaagaaggtgctgccctccagtgatgggaaaacccctgcagaaaagaaactgaaaccatcaactaacaccaaaaagaaagtttcgtttacaccaaatgaaccaggaaaatatacaaagttggaaaaagatgctctagatttgctctcagataatgaagaagaagatgcagaatcttcaatcttaacctttgaagaaagagatacttcatcgttaagcattgaggccagattggaatcaatagaggagaaattaagcatgatattagggctattaagaacactcaacattgctacagcaggacccacagcagcaagagatgggatcagagatgcaatgattggcgtaagagaggaattaatagcagacataataaaggaagccaaagggaaagcagcagaaatgatggaagaggaaatgagtcaacgatcaaaaataggaaacggtagtgtaaaactaacagagaaagcaaaagagcttaacaaaattgttgaagatgaaagcacaagtggagaatctgaagaagaagaagaaccaaaagacatacaagacaatagtcaagaagatgacatttaccagttaattatgtagtttaataaaaataaacaatgggacaagtaaaaatggagtcctacctagtagacacttatcaaggcattccttacacagcagctgttcaagttgatctaatagaaaaggacctgttacctgcaagcctaacaatatggttccctttgtttcaggccaacacaccaccagcagtgctgctcgatcagttgaaaaccctaacaataaccactctgtatgctgcatcacaaaatggtccaatactcaaagtgaatgcatcagcccaaggtgcagcaatgtctgtacttcccaaaaaatttgaagtcaatgcgactgtagcactcgatgaatatagcaaattggaatttgacaaactcacagtctgtgaagtaaaaacagtttacttaacaaccatgaaaccatacgggatggtatcaaaatttgtgagctcagccaaatcagttggcaaaaaaacacatgatctaatcgcactgtgtgattttatggatctagaaaagaacacacctgttacaataccagcattcatcaaatcagtttcaatcaaagagagtgagtcagctactgttgaagctgctataagcagtgaagcagaccaagctctaacacaggccaaaattgcaccttatgcgggattgattatgatcatgactatgaacaatcccaaaggcatattcaaaaagcttggagctgggactcaagttatagtagaactaggagcatatgtccaggctgaaagcataagtaaaatatgcaagacttggagccatcaagggacaagatatgtgttgaagtccagataacagccaagcaccttggccaagagctactaactctatctcatagattataaagtcaccattctagttatataaaaatcaagttagaacaagaattaaatcaatcaagaatgggacaaataaaaatgtcttggaaagtggtgatcattttttcattgttaataacacctcaacacggtcttaaagagagctatttagaagagtcatgtagcactataactgaaggatatctcagtgttctgaggacaggttggtataccaacgtttttacactggaggtaggtgatgtagagaaccttacatgtgctgatggacctagcttaataaaaacagaattagacctgaccaaaagtgcactaagagaactcagaacagtttctgctgatcaactggcaagagaggagcaaattgagaatcccagacaatctagatttgttctaggagcaatagcactcggtgttgcaacagcagctgcagttacagcaggtgttgcaattgccaaaaccatccggcttgaaagtgaagtaacagcaattaagaatgccctcaaaaagaccaatgaagcagtatctacattggggaatggagttcgagtgttggcaactgcagtgagggagctggaagattttgtgagcaagaatctaacacgtgcaatcaacaaaaacaagtgcgacattgctgacctgaaaatggccgttagcttcagtcaattcaacagaaggtttctaaatgttgtgcggcaattttcagacaatgctggaataacaccagcaatatccttggacttaatgacagatgctgaactagccagagctgtttccaacatgccaacatctgcaggacaaataaaactgatgttggagaaccgtgcaatggtaagaagaaaggggttcggaatcctgataggagtttacggaagctccgtaatttacatggtgcaactgccaatctttggagttatagacacgccttgctggatagtaaaagcggccccttcttgctcagaaaaaaagggaaactatgcttgccttttaagagaagatcaaggatggtattgtcagaatgcagggtcaactgtttactacccaaatgaaaaagactgcgaaacaagaggagaccatgtcttttgcgacacagcagcaggaatcaatgttgctgagcagtcaaaggagtgcaacatcaacatatccactactaattacccatgcaaagttagcacaggaagacaccctatcagtatggttgcactgtctcctcttggggctttggttgcttgctacaagggagtgagctgttccattggcagcaacagagtagggatcatcaagcaactgaacaaaggctgctcttatataaccaaccaagacgcagacacagtgacaatagacaacactgtataccagctaagcaaagttgagggcgaacagcatgttataaaaggaaggccagtgtcaagcagctttgatccagtcaaatttcctgaagatcaattcaatgttgcacttgaccaagttttcgaaagcattgagaacagtcaggccttggtggatcaatcaaacagaatcctaagcagtgcagagaaaggaaacactggcttcatcattgtaataattctaattgctgtccttggctctaccatgatcctagtgagtgtttttatcataataaagaaaacaaagaaacccacaggagcacctccagagctgagtggtgtcacaaacaatggcttcataccacataattagttaattaaaaataaagtaaattaaattaaaataaaataaaattaaaattaaaataaaataaaaataaaaatttgggacaaatcataatgtctcgcaaggctccatgcaaatatgaagtgcggggcaaatgcaatagaggaagtgagtgcaagtttaaccacaattactggagttggccagatagatacttactaataagatcaaattatttattaaatcaacttttaaggaacactgatagagctgatggcttatcaataatatcaggagcaggcagagaagataggacacaagattttgtcctaggttccaccaatgtggttcaaggttatattgatgataaccaaagcataacaaaagctgcagcctgttacagtctacataatataatcaaacaactacaagaagttgaagttaggcaggctagagataacaaaccatctgacagcaaacatgtggcacttcacaacttagtcctatcttatatggagatgagcaaaattcctgcatctttaatcaacaatctcaaaagactgccgagagagaaactgaaaaaattagcaaagcttataattgacttatcagcaggtgctgaaaatgactcttcatatgccttgcaagacagtgaaagcactaatcaagtgcagtgagcatggtcctgttttcattactatagaggttgattacatgatatggactcataaggacttaaaagaagctttatctaatgggatagtgaagtctcatactaacatttacaattgttatttagaaaacatagaaattatatatgtcaaggcttacttaagttagtaaaaacacatcagagtgggacaagtagttatggaggtgaaagtggagaacattcgaacaatagatatgctcaaagcaagagtgaaaaatcgtgtggcacgcagcaaatgctttaaaaatgcctctttgatcctaataggaataactacattgagtatagccctcaatatctatctaatcataaactatacaatgcaagaaaacacatccgaatcagaacatcacaccagctcatcacccatggaatccagcagggaaactccaacagtccctatggacaactcagacaccaatccaggctcacagtatccaactcaacagtccacagaaggctccacactctactttgcagcctcagcaagctcaccagagacagaaccaacatcaacaccagacacaacaagccgcccgcccttcgtcgacacacacacaacactaccaagtgcaagcagaacaaggacaagtccggcagtccacacaaaaaacaatccaaggacaagccccagaacacattccccaccatgggcaatgacaaggacggtccgtggaaccaccactctccgcacaagcagcacaagaaaaagaccgtccacagcatcagtccaacctgacagcagcgcaacaacccacaaacacgaagaagcaagcccagtgagcccgcaaacatctgcgagcacagcaagaccacaaaggaagggcatggaggccagcacatcaacaacatacaaccaaactagttaacaaaaaatacaaaataactctaagataaaccatatagacaccaacaattgagaagccaaaaggcaattcacaatctctccaaaaaggcaacaacaccatattagctccgcttaaatctccctggaaaaaacactcgcccatataccaactataccacaaccatcccaagaaaaaaagctgggtaaaacaacacccaagagacaaataacaatggatcctcttaatgaatccactgttaatgtctatcttcccgactcatatcttaaaggagtgatttcttttagtgagactaatgcaattggttcatgtctcttaaaaagaccttacctaaaaaatgacaacactgcaaaagttgccatagagaatcctgttatcgagcatgttagactcaaaaatgcaatcaattctaagatgaaaatatcagattacaggatagtagagccagtaaacatgcaacatgaaattatgaagaatgtacacagttgtgagctcacattattaaaacagtttttaacaaggagtaaaaatattagcactcttaaattaaatatgatatgtgattggctgcagttaaagtctacatcagatgatacctcaatcttaagttttatagatgtagaatttatacctagctgggtaagcaattggtttagtaattggtacaatctcaacaagttgattctggaattcaggaaagaagaagtaataagaactggttcaatcttgtgtaggtcattgggtaaattagtttttgttgtatcatcatacggatgtatagtcaagagcaacaaaagcaaaagagtgagcttcttcacatacaatcaactgttaacatggaaagatgtgatgttaagtagattcaatgcaaatttctgtatatgggtaagcaacagtctgaatgaaaatcaagaagggttagggttgagaagtaatctgcaaggcatattaactaataagctatatgaaactgtagattatatgcttagtttgtgttgcaatgaaggtttctcacttgtgaaagagtttgagggttttattatgagtgaaatccttaggattactgaacatgctcaattcagtactagatttagaaatactttattaaatggattaactgatcaattgacaaaattaaaaaataaaaacagactcagagttcatggtaccgtgttagaaaataatgattatccaatgtatgaagttgtacttaaattattaggagatactttgagatgtattaaattattaatcaataaaaacttagagaatgctgctgaattatactatatatttagaatattcggtcacccaatggtagatgaaagagatgcaatggatgctgtcaaattaaacaatgaaatcacaaaaatcctcaggttggagagcttgacagaactaagaggggcattcatattaaggattatcaaaggatttgtagacaacaacaaaagatggccgaaaattaaaaacttaaaagtgcttagtaaaagatggactatgtacttcaaagcaaaaagttaccctagtcaacttgaattaagtgaacaagattttttagagcttgctgcaatacagtttgaacaagagttttctgttcctgaaaaaaccaaccttgagatggtattaaatgataaagctatatcacctcctaaaagattaatttggtctgtgtacccaaaaaattacttacctgagacaataaaaaatcgatatctagaagagactttcaatgcaagtgatagtctcaaaacaagaagagtactagagtactatttgaaagataataaattcgaccaaaaagaacttaaaagttatgtggttaaacaagaatatttaaatgataaggatcatattgtctcgctaactggaaaagaaagagaattaagtgtaggtagaatgtttgctatgcaaccaggaaaacagcgacaaatacaaatattggctgaaaaattgttagctgataatattgtaccctttttcccagaaactttaacaaagtatggtgatctagatcttcagagaataatggaaatcaaatcagaactttcttctattaaaaccagaagaaatgatagttataataattacattgcaagagcatccatagtaacagatttaagtaagttcaaccaagcctttaggtatgaaactacagcgatctgtgcggatgtagcagatgaactacatggaacacaaagcctattctgttggttacatcttatcgttcctatgactacaatgatatgtgcctatagacatgcaccaccagaaacaaaaggtgaatatgatatagataagatagaagagcaaagtggtttatatagatatcatatgggtggtattgaaggatggtgtcaaaaactctggacaatggaagctatatctttattagatgttgtatctgtaaagacacgatgtcaaatgacatctttattaaacggtgacaaccaatcaatagatgtaagtaaaccagttaagttatctgagggtttagatgaagtgaaagcagattatagcttggctgtaaaaatgctaaaagaaataagagatgcatacagaaatataggccataaacttaaagaaggggaaacatatatatcaagagatcttcagtttataagtaaggtgattcaatctgaaggagtaatgcatcctacccctataaaaaagatcttaagagtgggaccatggataaacacaatattagatgacattaaaaccagtgcagagtcaatagggagtctatgtcaggaattagaatttaggggggaaagcataatagttagtctgatattaaggaatttttggctgtataatttatacatgcatgaatcaaagcaacaccccctagcagggaagcagttattcaaacaactaaataaaacattaacatcagtgcagagattttttgaaattaaaaaggaaaatgaagtagtagatctatggatgaacataccaatgcagtttggaggaggagatccagtagtcttctatagatctttctatagaaggacccctgattttttaactgaagcaatcagtcatgtagatattctgttaaaaatatcagccaacataagaaatgaagcgaaaataagtttcttcaaagccttactgtcaatagaaaaaaatgaacgtgctacactgacaacactaatgagagaccctcaagctgtgggctcagagcgacaagcaaaagtaacaagtgatatcaatagaacagcagttaccagcatcttaagtctttctccaaatcaacttttcagcgatagtgctatacactacagtagaaatgaagaagaggtcggaatcattgctgacaacataacacctgtttatcctcatggactgagagttttgtatgaatcattaccttttcataaagctgaaaaagttgtaaatatgatatcaggaacaaaatccataaccaacttattacagagaacatctgctattaatggtgaagatattgacagagctgtatccatgatgctggagaacctaggattattatctagaatattgtcagtagttgttgatagtatagaaattccaaccaaatctaatggtaggctgatatgttgtcagatatctagaaccctaagggagacatcatggaataatatggaaatagttggagtaacatcccctagcatcactacatgcatggatgtcatatatgcaactagctctcatttgaaagggataatcattgaaaagttcagcactgacagaactacaagaggtcaaagaggtccaaagagcccttgggtaggatcgagcacgcaagagaaaaaattagttcctgtttataacagacaaattctttcaaaacaacaaagagaacagctagaagcaattggaaaaatgagatgggtatataaagggacaccaggtttaagacgattactcaataagatttgtcttggaagtttaggcattagttacaaatgtgtaaaacctttattacctaggtttatgagtgtaaatttcctacacaggttatctgtcagtagtagacctatggaattcccagcatcagttccagcttatagaacaacaaattaccattttgacactagtcctattaatcaagcactaagtgaaagatttgggaatgaagatattaatttggtcttccaaaatgcaatcagctgtggaattagcataatgagtgtagtagaacaattaactggtaggagtccaaaacagttagttttaataccccaattagaagaaatagacattatgccaccaccagtgtttcaagggaaattcaattataagctagtagataagataacttctgaccaacatatcttcagtccagacaaaatagatatgttaacactggggaaaatgctcatgccaactataaaaggtcagaaaacagatcagttcttgaacaagagagagaattatttccatgggaacaatcttattgagtctttgtcagcagcgttagcatgtcattggtgtgggatattaacagagcaatgtatagaaaataatattttcaagaaagactggggtgacgggttcatatcggatcatgcttttatggacttcaaaatattcctatgtgtctttaaaactaaacttttatgtagttggggatcccaagggaaaaacattaaagatgaagatatagtagatgaatcgatagataaactgttaaggattgataatactttttggagaatgttcagcaaggttatgtttgaatcaaaggttaagaaaaggataatgttatatgatgtaaaatttctatcattagtaggttatatagggtttaagaattggtttatagaacagttgagatcagctgagttgcatgaggtaccttggattgtcaatgccgaaggtgatctggttgagatcaagtcaattaaaatctatttgcaactgatagagcagagtttatttttaagaataactgttttgaactatacagatatggcacatgctctcacaagattaatcagaaagaagttgatgtgtgataatgcactattaacttcaattccatccccaatggttaacttaactcaagttattgatcctacagaacaattagcttatttccctaagataacatttgaaaggctaaaaaattatgacactagttcaaattatgctaaaggaaagctaacaaggaattacatgatactgttgccatggcaacatgttaatagatataactttgtctttagttctactggatgtaaagttagtctaaaaacatgcattggaaaacttatgaaagatctaaaccctaaagttctgtactttattggagaaggggcaggaaattggatggccagaacagcatgtgaatatcctgacatcaaatttgtatacagaagtttaaaagatgaccttgatcatcattatcctttggaataccagagagtgataggagaattaagcaggataatagatagtggtgaagggctttcaatggaaacaacagatgcaactcaaaaaactcattgggatttgatacacagagtaagcaaagatgctttattaataactttatgtgatgcagaatttaaggacagagatgatttttttaagatggtaattctatggaggaaacatgtattatcatgcagaatttgcactacctatgggacagacctctatttattcgcaaagtatcatgctaaagactgcaatataaaattacctttttttgtgagatcagttgccacctttattatgcaaggtagtaaactgtcaggctcggaatgctacatactcttaacactaggccaccacaacaatttaccttgtcatggagaaatacaaaattctaagatgaaaatagcagcgtgtaatgatttttatgctgcaaaaaaacttgacaataaatcaattgaagccaactgtaaatcacttttatcagggctaagaataccgataaataagaaggaattaaatagacagagaaggttattaacactacaaagcaaccattcttctgtagcaacagttggaggtagcaaggtcatagagtctaaatggttaacaaacaaggcaaacacaataattgattggttagaacatattttaaattctccaaaaggtgaattaaattatgatttttttgaagcattagaaaatacttaccctaatatgattaaactaatagataatctagggaatgcagagattaaaaaactgatcaaagtaactggatatatgcttgtaagtaaaaaatgaaaaatgataaaaatgataaaataggtgacaacttcatactattccaaagtaatcatttgattatgcaattatgtaatagttaattaaaaactaaaaatcaaaagttaaaaactaataactgtcattaagtttattaaaaataagaaattataattggatgtatacggtttttttgccgt